SEQUENCE LISTING (19 sequences)

SEQ ID NO: 1 - DNA, 582 bp, synthetic construct
  source 1..582: mol_type = other DNA
atggagaaaaaaatcgatcaggtggatgttaaagattgcgccaaccacgaaatcaaaaaagttctggttccgggttgtcatggtagcgaaccgtgtattattcatcgtggtaaaccgtttcagctggaagcagtttttgaagcaaatcagaataccaaaaccgccaagattgaaatcaaagccagcattgatggtctggaagttgatgttccgggtattgatccgaatgcatgccactatatgaaatgtccgctggttaaaggtcagcagtatgatatcaaatatacctggaacgtgccgaaaattgcaccgaaaagcgaaaatgttgttgtgaccgttaaagtgatgggtgatgatggtgttctggcatgtgccattgcaacccatgcaaaaattcgtgatggatccggtcgtgcaaccacaggtggtaattggacaacccgtgccggtggtggtggagccagtccagcagctcctgcgcctggcggggactacaaagatgaagaccttcttggtggggcaagtccagcggcaccagcaccaggcggatccgcatggtcccaccctcaatttgaaaaataa

SEQ ID NO: 2 - DNA, 474 bp, synthetic construct
  source 1..474: mol_type = other DNA
atggagaaaaaaatcgatcaggtggatgttaaagattgcgccaaccacgaaatcaaaaaagttctggttccgggttgtcatggtagcgaaccgtgtattattcatcgtggtaaaccgtttcagctggaagcagtttttgaagcaaatcagaataccaaaaccgccaagattgaaatcaaagccagcattgatggtctggaagttgatgttccgggtattgatccgaatgcatgccactatatgaaatgtccgctggttaaaggtcagcagtatgatatcaaatatacctggaacgtgccgaaaattgcaccgaaaagcgaaaatgttgttgtgaccgttaaagtgatgggtgatgatggtgttctggcatgtgccattgcaacccatgcaaaaattcgtgatggatccggtcgtgcaaccacaggtggtaattggacaacccgtgccggtggtcatcaccatcatcaccattaa

SEQ ID NO: 3 - DNA, 453 bp, Arachis hypogaea
  source 1..453: mol_type = other DNA
cgtcagcagtgggaattacaaggagaccgccgctgccaaagtcaactggagcgcgccaatctgcgtccctgcgagcagcatttaatgcagaagatccagcgtgacgaggattcgtacgggcgtgacccctattctccgtctcaggacccatattcgccgtcacaagaccctgatcgtcgcgatccgtatagccctagcccgtatgaccgccgtggagcgggtagctcacaacaccaagagcgttgttgtaatgaattgaatgagttcgagaacaaccagcgttgtatgtgtgaggctttgcagcaaatcatggagaaccagtctgatcgccttcaaggacgtcagcaagagcagcaattcaaacgcgaactgcgtaatctgccgcaacagtgtggtcttcgtgcaccgcagcgttgtgacttagaggttgaatctggaggacgtgatcgttat

SEQ ID NO: 4 - DNA, 906 bp, Dermatophagoides pteronyssinus
  source 1..906: mol_type = other DNA
cgtcccagctcgattaaaacgtttgaggagtacaagaaagcatttaataagtcttacgccacctttgaggacgaagaagcagcccgcaaaaactttctggagtccgttaagtatgtacagagcaatggcggtgctattaaccacttgtccgacttatccttggatgagtttaagaaccgcttccttatgagcgccgaagcatttgagcaccttaagacacagtttgacctgaacgcagagactaacgcatgttccatcaacggtaatgcccctgcggaaattgacctgcgtcaaatgcgcactgtaacacccatccgtatgcaaggtggatgcggatcctgctgggcattttcaggcgtcgcggcgactgagtcagcttacttggcttatcgtaaccagtcgttagacctggctgagcaagaattggtggactgtgcttcgcaacacgggtgtcatggggatacgattccgcgcggaatcgaatacatccagcacaacggcgtcgtacaagaatcatactatcgctacgtggctcgtgagcaatcttgtcgtcgccccaacgcgcaacgtttcgggatttctaactattgccaaatttacccaccaaatgtgaataaaatccgtgaagcattagcccagacccattctgcgatcgctgtaatcattggaattaaagatttagacgctttccgtcattacgatgggcgtacaatcattcaacgcgacaatggttaccagccaaactatcacgcggtgaacattgtcggatattctaatgcgcagggggtagattattggatcgtgcgcaactcttgggacacaaattggggtgataacgggtacggctattttgcagccaacattgacttaatgatgatcgaagagtatccctatgtggttatctta

SEQ ID NO: 5 - DNA, 486 bp, Felis catus
  source 1..486: mol_type = other DNA
gttaagatggccgaaacctgcccgatcttttacgacgtctttttcgctgtagcgaacggaaacgaattgttattagacctgtcattgaccaaagtgggggcaacggaacctgaacgcactgcgatgaaaaaaatccaggactgctatgtggaaaacggtctgatttcgcgcgtccttgatgggcttgttatgacaactattagctccagtaaagactgtatgggagaagctgttcagaacacggtagaagacttgaagttgaataccctgggacgtgaaatctgtcctgccgtcaaacgcgatgtcgacttgttcttgacgggtacgcccgatgaatatgtcgaacaggtcgctcagtacaaggcccttccagtagtcttggaaaacgcccgcattctgaaaaactgtgttgatgctaagatgacggaagaagacaaggagaatgctttaagtgttttggataaaatctacacatctccgctgtgc

SEQ ID NO: 6 - DNA, 1113 bp, Ambrosia artemisiifolia
  source 1..1113: mol_type = other DNA
gcggaggatcttcaagagatccttcccgttaacgagactcgtcgcttgacgacatcgggggcgtataatatcatcgacgggtgctggcgtgggaaagctgattgggcagaaaaccgtaaggcgctggcggactgcgcacaggggttcggtaaaggcactgtgggcggcaaggacggcgacatctacactgttacgtccgacttggacgacgatgtagccaaccccaaggaaggcacgctgcgttttggtgcagcgcaaaatcgtccgctgtggattattttcgaacgcgacatggtgattcgtttggataaagaaatggtagtgaactcggataagactatcgacggacgcggggcgaaggtggagattatcaatgctggatttacgttaaacggtgtgaagaacgtgattattcacaatatcaatatgcacgacgtgaaggttaatcctggcggattgattaaaagcaacgatggtccggctgccccccgtgcggggtctgacggggacgccatctcaattagcggaagctctcaaatctggattgatcactgcagtttgtctaagagcgtagacggcttggtagatgctaagttgggcacgacgcgtcttaccgtttcgaatagcttgttcactcaacaccaattcgtacttctttttggtgctggggacgaaaatatcgaggatcgtggaatgctggcaacggtagcattcaatacatttaccgacaacgtggatcagcgtatgcctcgctgtcgtcatgggttttttcaagtagttaataacaactatgacaagtgggggagctacgctattggcggctcggcgtcaccaactatcttatcgcaggggaatcgtttttgtgcacccgatgagcgttcaaaaaagaatgttctgggacgtcatggtgaggccgctgctgagtccatgaagtggaactggcgcacgaacaaagacgtgctggagaacggcgccattttcgtcgcttcaggtgtggaccccgtgctgacaccagagcaaagcgccgggatgatcccagcagagcctggggagagtgcactgagtctgaccagcagcgccggcgtgctgagttgccaaccgggtgctccctgt

SEQ ID NO: 7 - DNA, 486 bp, Bos taurus
  source 1..486: mol_type = other DNA
ttaatcgtcactcaaaccatgaagggattggatatccagaaagttgccggaacgtggtactctctggctatggctgcatcggatattagtttactggatgcacagagcgctccgcttcgtgtctacgtggaagagctgaagccaacaccggaaggtgacttggagattcttttgcaaaaatgggagaacggcgaatgcgctcaaaagaaaattattgccgaaaaaaccaaaatcccggctgtattcaaaattgacgctttgaacgagaacaaagttttggtattggataccgattacaagaaatatctgttattctgtatggaaaattcagcggaacctgagcagtcgttagcctgtcagtgcttggtacgcaccccggaggtcgacgatgaagctcttgaaaagtttgacaaggcccttaaagccttgcctatgcacattcgtttgtcattcaatcctactcagttggaggagcagtgtcacatt

SEQ ID NO: 8 - DNA, 369 bp, Bos taurus
  source 1..369: mol_type = other DNA
gaacaactgactaaatgtgaagtttttcgtgaattaaaagaccttaaggggtatggaggcgtgagcctgcctgaatgggtttgcaccacatttcatactagcggatatgatacccaggcaatcgtacagaataacgacagcactgaatacggcttgttccaaatcaataataagatttggtgcaaggatgaccagaacccacacagctctaacatctgcaacattagctgtgataagtttttagacgacgatcttactgatgatattatgtgcgtcaagaaaattcttgacaaggttggcattaactactggttagcccacaaggcgttgtgctcagagaaattggaccaatggttgtgtgagaagttg

SEQ ID NO: 9 - DNA, 558 bp, Gallus gallus
  source 1..558: mol_type = other DNA
gccgaagtggactgttcccgttttcctggggcaacagataaagagggcaaagacgttctggtctgtaataaggaccttcgtcctatttgtggtacggatggcgtaacgtataccaatgactgtcttttgtgtgcctactctatcgagttcggcacaggcatctccaaggaacacgatggggaatgtaaagagactgttccaatgggctgttcctcctacgcaggaacaacgtctgaagatggcaaggtcatggttttatgcaaccgcgccttcaatcccgtatgcgggactgacggtgtgacatacgacaacgaatgcctgttgtgcgctcataaggttgaacaaggagcttccgttgataaacgccatgacggaggctgccgtaaagagctggccgcagtgagcgtggattgttcggaatatccaaaacctgactgcacggcagaagaccgtcccttgtgtggcagtgacaataaaacctacggcaataagtgtaatttttgcaacgcagttgttgagtccggcggtacgctgacattaagtcatttcggtaagtgc

SEQ ID NO: 10 - DNA, 1014 bp, Gallus gallus
  source 1..1014: mol_type = other DNA
tctacccgtacacagatcaataaggtggtccgcttcgataagttacctgggttcggcgattcaatcgaagcccaatgtggaacaagcgtcaacgttcattcaagtcttcgtgacattttgaatcagatcactaaaccgaatgatgtctactctttcagtcttgcgtcgcgtttatatgctgaagagcgttacccgatcttacccgagtatttacagtgcgtcaaagaattgtaccgcggaggactggaaccaatcaatttccaaacggccgccgaccaagcccgtgaattaattaatagttgggtggagtcacagactaacgggatcatccgtaacgtgttgcagccctcatcggtcgattcccaaactgccatggtattggtaaatgccattgtgtttaaggggttgtgggagaaagcctttaaggatgaggacacgcaggcgatgccattccgcgtaacggagcaggaaagcaagcctgtgcagatgatgtaccagatcggacttttccgcgtcgcttcgatggcgtctgagaagatgaaaatcttggaacttccctttgcatccggaaccatgtcgatgttagtgcttcttccggatgaagtctcgggattagagcagttggaaagtattattaatttcgagaaattgactgaatggacctcgtctaacgttatggaagaacgtaagattaaggtatatttaccccgcatgaaaatggaggagaaatataacttgacatcagtccttatggcgatgggtattacagacgtgttttcatcatccgcaaacttatcaggaatttcaagcgcggagtctttaaagatttcccaggcagtccacgctgcacatgcagaaattaatgaggccgggcgcgaggtcgtcggcagcgctgaggcgggcgttgatgcagcatctgttagcgaggaatttcgtgcagaccacccttttttgttctgcatcaaacacattgccaccaacgcggtattgtttttcggtcgttgtgtttccccc

SEQ ID NO: 11 - DNA, 387 bp, Gallus gallus
  source 1..387: mol_type = other DNA
aaggttttcggacgctgcgaattagcggctgctatgaagcgccatgggttagataattatcgcgggtactcgttaggtaattgggtctgtgcggctaagtttgaatctaacttcaatactcaagctacgaatcgcaacacggatggtagcactgattacggaattttgcagattaactctcgttggtggtgcaatgatggacgtacacctggaagtcgtaacctttgcaacattccgtgcagcgccctgttgtcgtccgatattacagcctcggtaaactgcgcgaagaaaattgtaagcgacgggaatgggatgaacgcctgggtagcatggcgcaaccgctgtaaaggaaccgacgtacaagcctggattcgcggatgccgtctg

SEQ ID NO: 12 - DNA, 984 bp, Periplaneta americana
  source 1..984: mol_type = other DNA
gttccattatataaattagttcacgtctttattaatacccaatatgcaggcattaccaagattggtaaccaaaattttctgaccgtttttgacagtacgtcttgcaatgtagtcgtggcatcacaagaatgtgttggcggtgcttgcgtatgcccgaatttgcaaaaatacgaaaaactgaaacctaagtatatcagtgatggaaatgtccaagttaaattttttgacacaggatcagctgtgggccgtggtattgaggattctttgacaattagtggtctgaccacctcacagcaagacatcgtattagcagatgagctgagccaggaagtgtgcatcttaagcgccgatgtggtcgtaggtatcgctgcacctggctgccccaacgctctgaagggaaagaccgttcttgaaaacttcgtcgaagaaaaccttattgcacccgtattctctattcaccacgcccgcttccaagatggcgagcatttcggtgaaatcatttttggtgggtcagattggaaatatgttgatggagagtttacttatgtgcctctggtgggggacgatagctggaaattccgccttgacggtgtgaaaattggggatactaccgttgccccggcaggtacccaagccatcatcgacactagcaaagcgattatcgtcggtccgaaagcgtacgtgaaccccatcaacgaagcaatcggttgcgtagtagagaagaccacgacacgccgtatctgtaaactggactgctctaagattccgtccttaccagatgtcacctttgtcattaatgggcgtaactttggcatttcatcgcagtactatattcagcaaaatggaaatctgtgctactcaggatttcagccttgcggccacagcgaccatttcttcatcggggacttctttgtggatcactactattctgagtttaactgggagggcaaaaccatgggttttggccgctccgtagaaagcgta

SEQ ID NO: 13 - DNA, 1059 bp, Cryptomeria japonica
  source 1..1059: mol_type = other DNA
gacaaccctatcgacagttgctggcgcggagatagcaactgggctcagaatcgcatgaagctggctgattgcgccgttggctttggtagttcaaccatggggggcaaagggggggacctttacacagtaacaaatagtgacgacgatcccgttaacccggcgcctgggaccttacgttatggtgccactcgtgatcgccctttatggatcatcttctcaggaaatatgaacattaaattgaagatgccaatgtatattgctggatacaaaacattcgatggacgtggagcccaggtatatattggaaatggaggcccatgtgtctttattaagcgcgtatccaatgtgatcatccatgggttatacttgtatggatgcagtacaagcgtgttggggaacgtgttaattaatgaaagtttcggcgttgaacctgtccacccccaggacggagatgcccttacactgcgtacagccacaaacatttggatcgatcacaatagtttctctggatcatcagatggacttgtagacgtgactcttacctcgacaggagtgaccattagtaacaatctgttttttaaccaccataaggtaatgttgttaggccacgatgatgcatatagcgatgataaatctatgaaagtaacagttgcttttaaccaatttggaccgaactgcggtcaacgtatgccccgtgcacgctacggcttggttcatgttgcgaataacaactacgatccttggactatctacgctatcggcggatcgagtaatcccacaattctgagcgaaggcaacagcttcactgctccgggggagtcctataagaagcaggttaccatccgcattggctgcaaaacctcatcatcatgtagcaattgggtgtggcaaagcacacaggatgttttttacaatggggcctacttcgtcagtagcggcaagtatgagggaggcaacatttatacaaagaaagaagcgtttaacgtagaaaacggaggagccactccacaattgacgaaaaatgcaggagtccttacgtgcagtttaagcaaacgttgt

SEQ ID NO: 14 - DNA, 489 bp, Betula pendula
  source 1..489: mol_type = other DNA
gaatttggcgtgttcaactatgaaaccgaaaccaccagtgttattccggcagcacgtctgtttaaagcatttattctggatggcgataacctgtttccgaaagttgcaccgcaggcaattagcagcgttgaaaatattgaaggtaatggtggtccgggtacgatcaaaaaaatcagctttccggaaggtctgccgttcaaatatgttaaagatcgtgttgatgaagtggaccacaccaacttcaaatataactatagcgtgattgaaggtggtccgattggtgataccctggaaaaaatctccaacgagattaaaatcgttgcaacaccggatggtggtagcattctgaaaattagcaacaagtatcacaccaaaggcgatcatgaagttaaagccgaacaggttaaagccagcaaagaaatgggtgaaaccctgctgcgtgcagttgaaagctatctgctggcacatagtgatgcatataatctgcaa

SEQ ID NO: 15 - DNA, 1353 bp, synthetic construct
  source 1..1353: mol_type = other DNA
atggagaaaaaaatccgcaccaaaattatcgcaattattccggcacgtagcggtagcaaaggtctgcgtaacaaaaatgcactgatgctgattgataaaccgctgctggcatataccattgaagcagcactgcagagcgaaatgtttgaaaaagttattgttaccaccgacagcgaacagtatggtgcaattgccgaaagctatggtgcagattttctgctgcgtccggaagaactggcaaccgataaagcaagcagctttgaatttatcaaacatgccctgagcatctataccgattatgaaaattttgcactgctgcagccgaccagtccgtttcgtgatagcacccatattattgaagccgtgaaactgtatcagaccctggaaaagtatcagtgtgttgttagcgttacccgtagcaataaaccgagccagattattcgtccgctggatgattatagcaccctgagcttttttgatctggactacagcaaatataaccgcaacagcattgttgaatatcatccgaatggtgccatttttatcgccaacaaacagcattatctgcataccaaacattttttcggtcgttatagcctggcctacatcatggataaagaaagctcactggatatcgatgatcgcatggattttgaactggccattaccattcagcagaagaaaaatcgtcagaagattctgtaccagaacatccataatcgcatcaacgaaaaacgcaacgaatttgatagcgtgagcgatattaccctgattggtcatagcctgtttgattattgggacgtgaaaaagatcaacgacatcgaagttaataacctgggtattgccggtatcaacagcaaagaatattacgagtacatcatcgagaaagagcgcattgttaattttggcgaattcgtgtttatctttttcggcaccaatgatattgttgtgagcgactggaaaaaagaggataccctttggtatctgaaaaagacctgccagtacatcaaaaaaaagaatgcagccagcaaaatctacctgctgagcgttccgcctgtttttggtcgtattgatcgtgataatcgtatcatcaatgacctgaatagctatctgcgtgaaaatgtggatttcgccaaatttatcagcctggatcatgttctgaaagattcctatggcaacctgaacaaaatgtatacctatgatggcctgcactttaacagcaatggttataccgttctggaaaacgaaattgccgagatcgttaaatggagccatccgcagttcgaaaaaggtggtggttctggtggtggttctggtggttctgcgtggagccatccgcagttcgaaaaataa

SEQ ID NO: 16 - DNA, 1341 bp, synthetic construct
  source 1..1341: mol_type = other DNA
atgcgcaccaaaattatcgcaattattccggcacgtagcggtagcaaaggtctgcgtaacaaaaatgcactgatgctgattgataaaccgctgctggcatataccattgaagcagcactgcagagcgaaatgtttgaaaaagttattgttaccaccgacagcgaacagtatggtgcaattgccgaaagctatggtgcagattttctgctgcgtccggaagaactggcaaccgataaagcaagcagctttgaatttatcaaacatgccctgagcatctataccgattatgaaaattttgcactgctgcagccgaccagtccgtttcgtgatagcacccatattattgaagccgtgaaactgtatcagaccctggaaaagtatcagtgtgttgttagcgttacccgtagcaataaaccgagccagattattcgtccgctggatgattatagcaccctgagcttttttgatctggactacagcaaatataaccgcaacagcattgttgaatatcatccgaatggtgccatttttatcgccaacaaacagcattatctgcataccaaacattttttcggtcgttatagcctggcctacatcatggataaagaaagctcactggatatcgatgatcgcatggattttgaactggccattaccattcagcagaagaaaaatcgtcagaagattctgtaccagaacatccataatcgcatcaacgaaaaacgcaacgaatttgatagcgtgagcgatattaccctgattggtcatagcctgtttgattattgggacgtgaaaaagatcaacgacatcgaagttaataacctgggtattgccggtatcaacagcaaagaatattacgagtacatcatcgagaaagagcgcattgttaattttggcgaattcgtgtttatctttttcggcaccaatgatattgttgtgagcgactggaaaaaagaggataccctttggtatctgaaaaagacctgccagtacatcaaaaaaaagaatgcagccagcaaaatctacctgctgagcgttccgcctgtttttggtcgtattgatcgtgataatcgtatcatcaatgacctgaatagctatctgcgtgaaaatgtggatttcgccaaatttatcagcctggatcatgttctgaaagattcctatggcaacctgaacaaaatgtatacctatgatggcctgcactttaacagcaatggttataccgttctggaaaacgaaattgccgagatcgttaaatggagccatccgcagttcgaaaaaggtggtggttctggtggtggttctggtggttctgcgtggagccatccgcagttcgaaaaataa

SEQ ID NO: 17 - DNA, 474 bp, synthetic construct; identical to SEQ ID NO: 2
  source 1..474: mol_type = other DNA

SEQ ID NO: 18 - protein, 6 aa, synthetic construct
GGNWTT

SEQ ID NO: 19 - protein, 10 aa, synthetic construct
ATTGGNWTTR

(19) United States
(12) Patent Application Publication
(10) Pub. No.: US 2026/0317803 A1
(43) Pub. Date: Oct. 15, 2026

(54) GLYCOCAP: A CELL-FREE GLYCOSYLATION PLATFORM FOR CLICKABLE AZIDO-SIALOGLYCAN PRODUCTION

(71) Applicant: Northwestern University, Evanston, IL (US)

(72) Inventors: Ariel Nicole Thames, Evanston, IL (US); Michael Christopher Jewett, Evanston, IL (US); Bruce Scott Bochner, Evanston, IL (US)

(21) Appl. No.: 19/113,720

(22) PCT Filed: Sep. 25, 2023
(86) PCT No.: PCT/US2023/075024

§ 371 Date: Mar. 20, 2025

Related U.S. Application Data
(60) Provisional application No. 63/376,869, filed on Sep. 23, 2022.

(51) Int. Cl. C12P 21/00; C12N 9/10; C12N 9/12

(52) U.S. Cl. CPC C12P 21/005; C12N 9/1051; C12N 9/1081; C12N 9/1241; C12Y 204/0109; C12Y 204/99001; C12Y 207/07043

(57) ABSTRACT

Disclosed herein are components, kits, platforms, and systems for novel, bacterial-based, cell-free glycosylation for incorporating non-canonical sugars and constructing azido-sialoglycoprotein structures.

### CROSS-REFERENCE TO RELATED PATENT APPLICATIONS

This application claims priority to U.S. application No. 63/376,869 filed on Sep. 23, 2022, the entire contents of which are incorporated by reference herein.

### STATEMENT REGARDING FEDERALLY SPONSORED RESEARCH OR DEVELOPMENT

This invention was made with government support under grant number AI65279 awarded by the National Institutes of Health, and grant number 1936789 awarded by the National Science Foundation, and grant HDTRA-1-20-10004 awarded by the Department of Defense. The government has certain rights in the invention.

### SEQUENCE LISTING

A Sequence Listing accompanies this application and is submitted as an XML file of the sequence listing named “702581_02410_Sequence_Listing.xml” which is 29,827 bytes in size and was created on Sep. 22, 2023. The sequence listing is electronically submitted via the Patent Center with the application and is incorporated herein by reference in its entirety.

### BACKGROUND

Lectins represent a class of promising therapeutic targets, but tools for making glycan-based therapeutics remain limited.

### SUMMARY

To address this need, the inventors developed a cell-free glycosylation platform for building clickable azido-sialoglycoproteins (GlycoCAP). A feature is the use of an Escherichia coli-based cell-free protein synthesis (CFPS) system that offers a blank slate for glycoengineering. By supplementing necessary building blocks and cofactors with glycosyltransferase-encoded plasmids, the inventors express a range of different bacterial glycosyltransferases to construct minimal glycan structures on a desired protein target. This system yields a highly homogeneous glycan population and allows supplementation of non-canonical sugars. As a model, the inventors constructed a minimal trisaccharide with a terminal azido-sialic acid. Through a series of optimizations, the inventors achieved >60% sialylation efficiency with a non-canonical azido-sialic acid. The azido group on the sialic acid serves as a handle for click chemistry conjugation for optimizing lectin binding pocket interactions with small chemical modifications of the glycan. The disclosed technology and platform will grant access to a wide range of glycan structures that can be used to therapeutically target lectins in a range of disease applications.

Accordingly, in one aspect a method for preparing an azido-sialoglycoprotein is provided. The method can include, in a cell-free in vitro glycosylation assembly (IVA) reaction, providing a polypeptide comprising an amino acid sequence recognized by ApNGT for glycosylation; wherein the IVA comprises one or more of: ApNGT, LgtB, CSTI, PdST6, NeuA, or CTP.

In another aspect, a composition and/or kit is provided. The composition and/or kit can include an in vitro glycosylation assembly (IVA), where the IVA comprises one or more of: ApNGT, LgtB, CSTI, PdST6, NeuA, or CTP.

In yet another aspect, a genetically engineered E. coli strain is provided. The genetically engineered E. coli strain can include one or more nucleic acid sequences encoding one or more of: ApNGT, LgtB, CSTI, PdST6, or NeuA.

### BRIEF DESCRIPTION OF THE FIGURES

FIGS. 1A, 1B, and 1C. GlycoCAP platform: a cell-free glycosylation platform for building clickable azido-glycoproteins. (FIG. 1A) Plasmids encoding each component of the glycosylation pathway are supplemented in lysate harvested from E. coli cells along with necessary cofactors and building blocks for transcription and translation. After overnight expression, cell-free expressed components are mixed in optimized proportions along with sugar substrates and cofactors in an in vitro glycosylation (IVA) reaction. (FIG. 1B) Components of the pathway include (i) a target glycoprotein designed with an amino acid sequence recognized by (ii) ApNGT for modification with glucose by N-linked glycosylation; (iii) LgtB elaborates galactose onto glucose in a β1,4 linkage, and either (iv) CSTI or PdST6 can be added to elaborate a sialic acid on galactose in an α2,3 or α2,6 linkage, respectively. Finally, (v) NeuA is required to convert noncanonical sugars, such as C5-azido-sialic acid or C9-azido-sialic acid, into activated sugar substrates for CSTI/PdST6 by conjugating the sugars to a phosphonucleotide. (FIG. 1C) Soluble concentration of each component in the CFPS reaction after expression is detected by radioactive incorporation of [14C]-leucine (n=3; error bars=standard deviation).

FIGS. 2A, 2B, and 2C. Optimization of GlycoCAP for noncanonical sugar incorporation. (FIG. 2A) IVA reactions were assembled based on optimized ratios (i.e., where 13% of the final reaction volume was made up of either crude lysate expressing PdST6 if an α2,6 linkage was desired or crude lysate expressing CSTI if an α2,3 linkage was desired) to achieve glycosylation when activated CMP-sialic acid is supplemented directly and NeuA is not required to install sialyllactose onto the target glycoprotein. Under these conditions, 67±7% glycosylation efficiency is achieved (n=2; error bars=average error). The color-coded legend applies to all bar graphs shown. (FIG. 2B) Plasmid encoding NeuA was designed for CFPS. Once expressed, NeuA was assembled into the IVA reaction with its needed substrates: nonactivated sialic acid and CTP. Titration of NeuA was performed to determine 2 μL (which corresponds to 4.5% volume) as the optimal volume to add to the IVA assembly (n=4; error bars=standard deviation). An additional titration of standard sialic acid shows that increasing [sialic acid] to 7.5 mM and [CTP] to 45 μM enhances the glycosylation efficiency to 64±5% (n=4; error bars=standard deviation). (FIG. 2C) Titration of noncanonical sialic acids shows that C9-azido-sialic acid is as efficiently incorporated as standard sialic acid, with 7.5 mM sialic acid and 45 μM CTP being the optimal concentration to achieve 69±8% efficiency. A concentration of 125 mM C5-azido-sialic acid is required to achieve the optimal glycosylation efficiency of 29±1% (n=2; error bars=average error). All glycosylation efficiencies are determined by MS of trypsinized samples and estimated by the area under the curve of each pathway intermediate's extracted ion chromatogram based on the theoretical mass of the resulting glycopeptide.

FIGS. 3A, 3B, and 3C. Structures made with optimized conditions of GlycoCAP. (FIG. 3A) Chemical structures installed onto proteins in GlycoCAP. (FIG. 3B) Efficiencies for each species are represented as a bar graph (n=2; error bars=average error). (FIG. 3C) MS confirmation of each species is shown in relation to the peaks of its pathway intermediates in the +2 charge state. MS data show a representative spectra selected from n=2 experiments.

FIGS. 4A and 4B. Click chemistry conjugation of azido-sialoglycoproteins. (FIG. 4A) Conjugation by click chemistry with either a strained alkyne (top) or standard alkyne (bottom) is possible with the azido-sialoglycan structures built by GlycoCAP. SA=sodium ascorbate. (FIG. 4B) Fluorescent gel demonstrates successful click conjugation onto a C9-azido-sialoglycoprotein with either a fluorescent strained alkyne (DBCO 545) by strain-promoted click chemistry (lane 3) or a fluorescent standard alkyne (AF546) by copper-catalyzed click chemistry (lane 4). Fluorescent bands are visible at the expected masses for Der p 2 in its monomer form (~17 kDa) and dimer form (~34 kDa) except in the lane where azido-sialoglycoprotein was loaded without any fluorophore conjugation as a negative control (lane 2). Gel representative of n=3 experiments.

FIG. 5. Total and soluble cell-free expression of pathway enzymes. Total and soluble expression of each enzyme is quantified by incorporation of radioactive [14C]-leucine (n=3; error bars=standard deviation). ApNGT, LgtB, and PdST6 demonstrate a high degree of relative soluble expression to total expression, where NeuA and CSTI have a lesser degree of relative soluble expression. Only the soluble portion is assembled into in vitro glycosylation reactions and all pathway enzymes have sufficient solubility and activity for the GlycoCAP platform.

FIGS. 6A and 6B. NeuA DNA expression and glycosylation with and without a CAT linker. NeuA was designed with and without a 5′ CAT linker nucleotide sequence, which is often used to improve expression. (FIG. 6A) Both versions of NeuA expressed similarly as determined by incorporation of radioactive [14C]-leucine (n=3; error bars=standard deviation). (FIG. 6B) Both versions of NeuA also led to similar glycosylation efficiencies at equivalent titration volumes (n=2; errors bars=average error). Therefore, the CAT linker is not necessary to optimize NeuA expression and function in the GlycoCAP platform.

FIG. 7. ¹H and ¹³C NMR spectra of synthesized N-azidoacetylneuraminic acid (C5-azido sialic acid) in D₂ (500 MHz).

FIGS. 8A and 8B. Coomassie-stained protein gel (FIG. 8A) and extracted ion chromatograms (EICs) of 1 mL IVA azido-sialoglycoprotein products (FIG. 8B). Area under the curve of each EIC estimates relative abundance of each pathway intermediate and is used to calculate glycosylation efficiency.

FIG. 9. C9-azido sialic acid demonstrates E. coli cell toxicity. E. coli cells were supplemented with increasing [C9-azido sialic acid] from left to right (0.25, 0.5, 1, 1.5, 2, 2.5 mM) in otherwise equivalent culture conditions. Decreasing optical densities were observed for cultures incubated with increasing [C9-azido sialic acid], indicating some degree of cellular toxicity caused by the compound.

FIGS. 10A and 10B. Optimization of base pathway for Example 2. An optimal set of conditions was used to establish a benchmark for yields based on “base conditions” without the added step of NeuA activation of sialic acid. (FIG. 10A) With initial conditions, the sialylated final product represents ~20% of the total reaction products. The remaining 80% is comprised of the precursor reaction intermediates. (FIG. 10B) After a series of optimizations that includes (i) flash freezing LgtB after 6 hours of expression to prevent degradation products from protease activity (ii) adding flanking linker sequences to the Glyctag, and (iii) reducing precipitation induced by the HEPES buffer, the reaction yields a final product proportion of ~70% for the pathway with CSTI and ~60% for the pathway with PdST6.

FIGS. 11A and 11B. Optimization of NeuA Addition to Base Pathway for Example 2. A titration of CFPS lysate expressing NeuA was performed to determine the amount that achieves highest yields of sialylated product. (FIG. 11A) When 2 uL of the NeuACFPS reaction is added to the IVA containing CSTI pathway components, sialylated product makes up ~60% of the reaction product. (FIG. 11B) 5 uL of CFPS lysate expressing NeuA is required to achieve optimal yields in the IVA containing PdST6 pathway components (~40%). These yields are very high, but do not quite match the yields of base case conditions.

FIG. 12. NeuA does not require a CAT linker for optimal activity. Yields of sialylated product do not vary significantly with equivalent volumes of CFPS lysate when NeuA is designed with a CAT linker (right) or without (left). This is true in both the CSTI (top) and PdST6 (bottom) pathways IVA's.

FIGS. 13A and 13B. Supplementation of additional sugar and nucleotide substrate matches yields to “base conditions.” With addition of 2 uL of CFPS lysate expressing NeuA and triple the concentration of sialic acid and CTP (to 7.5 mM) in IVA, the yield of sialylated product approaches the optimal value in “base conditions” in IVA's containing either CSTI pathway (FIG. 13A) components or PdST6 pathway components (FIG. 13B).

FIGS. 14A and 14B. Titration of Non-Canonical Sialic Acid in IVA with CSTI Pathway. (FIG. 14A) Azido-sialic acid was supplemented into the IVA in place of standard sialic acid to allow for incorporation of this clickable sialic acid into the glycan structure. (FIG. 14B) The azido-sialic acid had an azide group either at the carbon 9 position (left) or carbon 5 position (right). Supplementation of C9-azido sialic acid at 2× the concentration of standard sialic acid promotes highest yield of sialylated product. Supplementation of C5-azido sialic acid at 10× the concentration yields highest yield of sialylated product. Yields are much lower for incorporation of C5-azido sialic acid than for C9-azido sialic acid, presumably due to reduced permissivity of NeuA, or CSTI to the additional chemical group at this position of its substrate.

FIGS. 15A and 15B. Titration of Non-Canonical Sialic Acid in IVA with PdST6 Pathway. (FIG. 15A) Azido-sialic acid was supplemented into the IVA in place of standard sialic acid to allow for incorporation of this clickable sialic acid into the glycan structure. (FIG. 15B) The azido-sialic acid had an azide group either at the carbon 9 position (left) or carbon 5 position (right). Supplementation of C9-azido sialic acid at 3× the concentration of standard sialic acid promotes highest yield of sialylated product in the PAST6 pathway. Supplementation of C5-azido sialic acid at 10× the concentration yields highest yield of sialylated product. Yields are much lower for incorporation of C5-azido sialic acid than for C9-azido sialic acid, presumably due to reduced permissivity of NeuA, or PdST6 to the additional chemical group at this position of its substrate.

FIG. 16. Higher supplementation of C5-azido sialic acid improves yields of sialylated product. Supplementation of C5-azido sialic acid at 50× the concentration of standard sialic acid in base conditions yields the highest yield of sialylated product in the CSTI pathway.

FIG. 17. Higher supplementation of C5-azido sialic acid improves yields of sialylated product. Supplementation of C5-azido sialic acid at 50× the concentration of standard sialic acid in base conditions yields the highest yield of sialylated product in the PAST6 pathway.

FIGS. 18A and 18B. Siglec recognition of GlycoCAP installed glycan. (FIG. 18A) An AlphaLISA assay was used to assess binding of Siglec-3 (CD33) to the sialoglycan installed with the GlycoCAP platform. The AlphaLISA involves a donor bead that associates with one binding partner via an affinity tag and an acceptor bead that associates with the other binding partner. If the two entities bind, they will bring the beads in close proximity and a energy transfer will occur causing an emission of light that can be read on a plate reader. (FIG. 18B) CD33 and Der p 2 were co-titrated to determine optimal concentration for detection of binding. At 50 nM CD33 and 12.5 nM α2,6-sialoglyco-Der p 2, there is the highest signal over background indicating that CD33 does indeed recognize the glycan and does so at these optimal concentrations. This experiment confirms that the glycan installed retains expected functional activity.

FIGS. 19A, 19B, and 19C. Click conjugation of clickable azido-sialoglycoprotein product from GlycoCAP. (FIG. 19A) An alkyne-containing fluorophore was conjugated to the azido-sialoglycorprotein and detected on a fluorescent gel. (FIG. 19B) Lane 1=ladder, lane 2=negative control, lane 3=positive control, lane 4=ladder, lane 5=GlycoCAP-constructed azido-sialoglycoprotein with no alkyne-containing moiety provided, lane 6=GlycoCAP-constructed azido-sialoglycoprotein with a strained alkyne (DBCO)-containing fluorophore provided, lane 7=GlycoCAP-constructed azido-sialoglycoprotein with a standard alkyne-containing fluorophore provided. The presence of a fluorescent band at the expected mass (~70 kDa for α-crystalline and 17 kDa for C9-azidosialoglyco-Der p 2) when a fluorophore is provided indicates successful click conjugation of the fluorophore onto the protein. The band at ~34 kDa indicates the presence of dimer formation, which appears to be enhanced by the conditions for the click chemistry reactions with the standard alkyne. (FIG. 19C) Click chemistry conjugation with either a standard (top) or strained (DBCO) (bottom) alkyne results in conjugation of the azide-containing group and alkyne-containing group with the intervening structures shown.

FIG. 20. Peak lists used to generate mass spectra. Yellow highlight=species of interest. Red=contaminating species carried over from purification. Intensity of species in red was manually reduced (1% deconvolute column) to eliminate noise and emphasize peaks of interest in final spectra.

### DETAILED DESCRIPTION

Lectins represent a class of promising therapeutic targets, but tools for making glycan-based therapeutics remain limited. To address this need, a cell-free glycosylation platform for building clickable azido-sialoglycoproteins (GlycoCAP) has been developed herein. A key feature is the use of an Escherichia coli-based cell-free protein synthesis (CFPS) system that offers a blank slate for glycoengineering. By supplementing necessary building blocks and cofactors with glycosyltransferase-encoded plasmids, a range of different bacterial glycosyltransferases can be expressed to construct minimal glycan structures on a desired protein target. This system yields a highly homogeneous glycan population and allows supplementation of non-canonical sugars. As an example, a minimal trisaccharide with a terminal azido-sialic acid was constructed. Through a series of optimizations, >60% sialylation efficiency with a non-canonical azido-sialic acid has been achieved. The azido group on the sialic acid serves as a handle for click chemistry conjugation for optimizing lectin binding pocket interactions with small chemical modifications of the glycan. It is anticipated that this platform will grant access to a wide range of glycan structures that can be used to therapeutically target lectins in a range of disease applications.

Disclosed herein are novel, bacterial-based, cell free glycosylation platforms for incorporating non-canonical sugars and constructing azido-sialoglycoprotein structures. This platform also offers a novel approach for conjugating different species to a protein on an entity separate from the protein itself. This approach can be used toward the application of many diseases and can be used to discover Siglec and lectin ligands and develop lectin-targeting therapeutics for the treatment of allergy, and for the prevention of anaphylaxis.

The technology disclosed herein provides a commercially friendly platform for constructing non-canonical glycans on proteins with (1) high homogeneity, (2) site specificity, (3) and high efficiency. This invention provides a novel tool that would open up a new approach for discovering Siglec and lectin ligands, which have relevance in allergy, autoimmunity, blood cancers, mast cell and eosinophil-driven diseases, and neurological diseases, among others. The technology disclosed herein also provides a new approach for protein conjugation which would offer new strategies for protein labeling, development of bi-specific therapeutics, and protein localization.

### Exemplary Applications and Advantages

Current strategies for glycoprotein production often rely on mammalian cells, which yield heterogeneous glycan populations and are low efficiency, or chemo-enzymatic synthesis, which is difficult to scale up. These strategies have more stringent parameters for reaction conditions, which may exclude the addition and incorporation of non-canonical sugars. The blank slate bacterial platform and open reaction environment of a cell free system allow for the site-specific construction glycans on proteins with easy supplementation of noncanonical sugars, yielding homogeneous non-canonical glycoprotein production and easy scale up. This platform not only addresses challenges in glycosylation of proteins, but also provides a novel handle for conjugating two constructs of interest.

Existing tools for conjugating moieties (chemical groups, nucleotides, proteins, carbohydrates, fluorophores, labels) to proteins relies on chemistry in amine or thiol-containing amino acids (which is less efficient and doesn't grant site-specific control), direct chemical synthesis (which is complex and costly), or via non-canonical amino acids (which may disrupt protein folding and structure). This platform provides a conjugation handle by incorporation of an azido-containing glycan that can be incorporated site-specifically and is separate from the protein, allowing protein structure to remain intact. It also relies on a conjugation method that is highly efficient, but relies on relatively simple reaction conditions.

Exemplary applications of the disclosed technology include, but are not limited to the following: Siglec ligand discovery, lectin ligand discovery, development of siglec-targeting therapeutics, development of lectin-targeting therapeutics, metabolic labelling of glycoprotein, warhead conjugation of glycoprotein, conjugation of proteins for bi-specific therapeutics.

Exemplary advantages of the disclosed technology includes, but is not limited to the following: E. coli lack native glycosylation machinery, so there is no competition for glycosylation sites or machinery and E. coli provide a blank slate for glycoengineering; A cell free system allows an open reaction environment, allowing for addition of non-canonical sugars without concern for effects on living cells; The open reaction environment also gives a tunable system for non-canonical glycoprotein production condition optimizations; The bacterial system yields highly homogeneous glycan populations; Control over DNA design enables site-specific glycosylation, allowing control over location and over valency; This platform also grants easy scale up for manufacturing; Click chemistry is a highly efficient conjugation method.

### Definitions and Terminology

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a gene” or “an oligosaccharide” should be interpreted to mean “one or more genes” and “one or more oligosaccharides,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the terms “bind,” “binding,” “interact,” “interacting,” “occupy” and “occupying” refer to covalent interactions, noncovalent interactions and steric interactions. A covalent interaction is a chemical linkage between two atoms or radicals formed by the sharing of a pair of electrons (a single bond), two pairs of electrons (a double bond) or three pairs of electrons (a triple bond). Covalent interactions are also known in the art as electron pair interactions or electron pair bonds. Noncovalent interactions include, but are not limited to, van der Waals interactions, hydrogen bonds, weak chemical bonds (via short-range noncovalent forces), hydrophobic interactions, ionic bonds and the like. A review of noncovalent interactions can be found in Alberts et al., in Molecular Biology of the Cell, 3d edition, Garland Publishing, 1994. Steric interactions are generally understood to include those where the structure of the compound is such that it is capable of occupying a site by virtue of its three-dimensional structure, as opposed to any attractive forces between the compound and the site.

### Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem, and Mol. Biol. 26 (3/4): 227-259; and Owczarzy et al., 2008, Biochemistry, 47:5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

Oligonucleotides and polynucleotides may optionally include one or more non-standard nucleotide(s), nucleotide analog(s) and/or modified nucleotides. Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

As utilized herein, a “deletion” means the removal of one or more nucleotides relative to the native polynucleotide sequence. The engineered strains that are disclosed herein may include a deletion in one or more genes (e.g., a deletion in gmd and/or a deletion in waaL). Preferably, a deletion results in a non-functional gene product. As utilized herein, an “insertion” means the addition of one or more nucleotides to the native polynucleotide sequence. The engineered strains that are disclosed herein may include an insertion in one or more genes (e.g., an insertion in gmd and/or an insertion in waaL). Preferably, a deletion results in a non-functional gene product. As utilized herein, a “substitution” means replacement of a nucleotide of a native polynucleotide sequence with a nucleotide that is not native to the polynucleotide sequence. The engineered strains that are disclosed herein may include a substitution in one or more genes (e.g., a substitution in gmd and/or a substitution in waaL). Preferably, a substitution results in a non-functional gene product, for example, where the substitution introduces a premature stop codon (e.g., TAA, TAG, or TGA) in the coding sequence of the gene product. In some embodiments, the engineered strains that are disclosed herein may include two or more substitutions where the substitutions introduce multiple premature stop codons (e.g., TAATAA, TAGTAG, or TGATGA).

In some embodiments, the engineered strains disclosed herein may be engineered to include and express one or heterologous genes. As would be understood in the art, a heterologous gene is a gene that is not naturally present in the engineered strain as the strain occurs in nature. A gene that is heterologous to E. coli is a gene that does not occur in E. coli and may be a gene that occurs naturally in another microorganism (e.g. a gene from C. jejuni) or a gene that does not occur naturally in any other known microorganism (i.e., an artificial gene).

### Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl) alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 21ufa21hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≥100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

As used herein, “translation template” refers to an RNA product of transcription from an expression template that can be used by ribosomes to synthesize polypeptides or proteins.

The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture.

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Cell-Free Protein Synthesis (CFPS)

The strains and systems disclosed herein may be applied to cell-free protein synthesis methods as known in the art. See, for example, U.S. Pat. Nos. 5,478,730; 5,556,769; 5,665,563; 6,168,931; 6,869,774; 6,994,986; 7,118,883; 7,189,528; 7,338,789; 7,387,884; 7,399,610; 8,703,471; and 8,999,668. See also U.S. Published Application Nos. 2015-0259757, 2014-0295492, 2014-0255987, 2014-0045267, 2012-0171720, 2008-0138857, 2007-0154983, 2005-0054044, and 2004-0209321. See also U.S. Published Application Nos. 2005-0170452; 2006-0211085; 2006-0234345; 2006-0252672; 2006-0257399; 2006-0286637; 2007-0026485; 2007-0178551; and 2018-0016612. See also Published PCT International Application Nos. 2003/056914; 2004/013151; 2004/035605; 2006/102652; 2006/119987; and 2007/120932. See also Jewett, M. C., Hong, S. H., Kwon, Y. C., Martin, R. W., and Des Soye, B. J. 2014, “Methods for improved in vitro protein synthesis with proteins containing non standard amino acids,” U.S. Patent Application Ser. No. 62/044,221; Jewett, M. C., Hodgman, C. E., and Gan, R. 2013, “Methods for yeast cell-free protein synthesis,” U.S. Patent Application Ser. No. 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No. 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No. 62/098,578. See also Guarino, C., & DeLisa, M. P. (2012). A prokaryote-based cell-free translation system that efficiently synthesizes glycoproteins. Glycobiology, 22 (5), 596-601. The contents of all of these references are incorporated in the present application by reference in their entireties.

In certain exemplary embodiments, one or more of the methods described herein are performed in a vessel, e.g., a single, vessel. The term “vessel,” as used herein, refers to any container suitable for holding on or more of the reactants (e.g., for use in one or more transcription, translation, and/or glycosylation steps) described herein. Examples of vessels include, but are not limited to, a microtitre plate, a test tube, a microfuge tube, a beaker, a flask, a multi-well plate, a cuvette, a flow system, a microfiber, a microscope slide and the like.

In certain exemplary embodiments, physiologically compatible (but not necessarily natural) ions and buffers are utilized for transcription, translation, and/or glycosylation, e.g., potassium glutamate, ammonium chloride and the like. Physiological cytoplasmic salt conditions are well-known to those of skill in the art.

The strains and systems disclosed herein may be applied to cell-free protein methods in order to prepare glycosylated macromolecules (e.g., glycosylated peptides, glycosylated proteins, and glycosylated lipids). Glycosylated proteins that may be prepared using the disclosed strains and systems may include proteins having N-linked glycosylation (i.e., glycans attached to nitrogen of asparagine and/or arginine sidechains) and/or O-linked glycosylation (i.e., glycans attached to the hydroxyl oxygen of serine, threonine, tyrosine, hydroxylysine, and/or hydroxyproline). Glycosylated lipids may include O-linked glycans via an oxygen atom, such as ceramide.

The glycosylated macromolecules disclosed herein may include unbranched and/or branched sugar chains composed of monomers as known in the art such as glucose (e.g., β-D-glucose), galactose (e.g., β-D-galactose), mannose (e.g., β-D-mannose), fucose (e.g., α-L-fucose), N-acetyl-glucosamine (GlcNAc), N-acetyl-galactosamine (GalNAc), neuraminic acid, N-acetylneuraminic acid (i.e., sialic acid), and xylose, which may be attached to the glycosylated macromolecule, growing glycan chain, or donor molecule (e.g., a donor lipid and/or a donor nucleotide) via respective glycosyltransferases (e.g., oligosaccharyltransferases, GlcNAc transferases, GalNAc transferases, galactosyltransferases, and sialyltransferases). The glycosylated macromolecules disclosed herein may include glycans as known in the art including but not limited to Man₃GlcNAc₂ glycan, Man₅GlcNAc₃ glycan, and the fully sialylated human glycan Man₃GlcNAc₄Gal₂Neu₅Ac₂. As such, the disclosed engineered strains may be enriched in glycans and/or lipid-linked oligosaccharides (LLOs) such as, but not limited to, Man₃GlcNAc₂ glycan, Man₅GlcNAc₃ glycan, and/or Man₃GlcNAc₄Gal₂Neu₅Ac₂ glycan, and the engineered strains may be utilized to prepare lysates that are enriched in glycans and/or lipid-linked oligosaccharides (LLOs) such as, but not limited to, Man₃GlcNAc₂ glycan, Man₅GlcNAC₃ glycan, and/or Man₃GlcNAc₄Gal₂Neu₅Ac₂ glycan.

The disclosed cell-free protein synthesis systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

Cell-Free Glycoprotein Synthesis (CFGpS) in Prokaryotic Cell Lysates Enriched with Components for Glycosylation

Disclosed are compositions and methods for performing cell-free glycoprotein synthesis (CFGpS). In some embodiments, the composition and methods include or utilize prokaryotic cell lysates enriched with components for glycosylation and prepared from genetically modified strains of prokaryotes. Compositions and methods for performing cell-free glycoprotein synthesis (CFGpS) and for in vitro synthesis of bioconjugates and uses thereof (e.g., as vaccines) via recombinant production of N-glycosylated proteins in prokaryotic lysates are known in the art. (See, e.g., U.S. Published Application No. 2018-0016612, the content of which is incorporated herein by reference in its entirety). Disclosed herein are improved compositions and methods for performing cell-free glycoprotein synthesis (CFGpS).

In some embodiments, the genetically modified prokaryote is a genetically modified strain of Escherichia coli or any other prokaryote suitable for preparing a lysate for CFGpS. Optionally, the modified strain of Escherichia coli is derived from rEc.C321. IN some embodiments, the modified strain includes genomic modifications (e.g., deletions of genes rendering the genes inoperable) that preferably result in lysates capable of high-yielding cell-free protein synthesis. Also, in some embodiments, the modified strain includes genomic modification (e.g., deletions of genes rendering the genes inoperable) that preferably result in lysates comprising sugar precursors for glycosylation at relatively high concentrations (e.g., in comparison to a strain not having the genomic modification). In some embodiments, a lysate prepared from the modified strain comprises sugar precursors at a concentration that is at least 20%, 30%, 40%, 50%, 60%, 70%, 80%, 90%, 100%, 150%, 200%, or higher than a lysate prepared from a strain that is not modified.

In some embodiments, the modified strain includes a modification that results in an increase in the concentration of a monosaccharide utilized in glycosylation (e.g., glucose, mannose, N-acetyl-glucosamine (GlcNAc), N-acetyl-galactosamine (GalNAc), galactose, sialic acid, neuraminic acid, fucose). As such, the modification may inactivate an enzyme that metabolizes a monosaccharide or polysaccharide utilized in glycosylation. In some embodiments, the modification inactivates a dehydratase or carbon-oxygen lyase enzyme (EC 4.2) (e.g., via a deletion of at least a portion of the gene encoding the enzyme). In particular, the modification may inactivate a GDP-mannose 4,6-dehydratase (EC 4.2.1.47). When the modified strain is E. coli, the modification may include an inactivating modification in the gmd gene (e.g., via a deletion of at least a portion of the gmd gene).

In some embodiments, the modified strain includes a modification that inactivates an enzyme that is utilized in the glycosyltransferase pathway. In some embodiments, the modification inactivates an oligosaccharide ligase enzyme (e.g., via a deletion of at least a portion of the gene encoding the enzyme). In some embodiments, the modification may inactivate an O-antigen ligase that optionally conjugates an O-antigen to a lipid A core oligosaccharide. The modification may include an inactivating modification in the waaL gene (e.g., via a deletion of at least a portion of the waaL gene).

In some embodiments, the modified strain includes a modification that inactivates a dehydratase or carbon-oxygen lyase enzyme (e.g., via a deletion of at least a portion of the gene encoding the enzyme) and also the modified strain includes a modification that inactivates an oligosaccharide ligase enzyme (e.g., via a deletion of at least a portion of the gene encoding the enzyme). The modified strain may include an inactivation or deletion of both gmd and waaL.

In some embodiments, the modified strain may be modified to express one or more orthogonal or heterologous genes. In particular, the modified strain may be genetically modified to express an orthogonal or heterologous gene that is associated with glycoprotein synthesis such as a glycosyltransferase (GT) which is involved in the lipid-linked oligosaccharide (LLO) pathway. In some embodiments, the modified strain may be modified to express an orthogonal or heterologous oligosaccharyltransferase (EC 2.4.1.119) (OST). Oligosaccharyltransferases or OSTs are enzymes that transfer oligosaccharides from lipids to proteins.

In some embodiments, the modified strain may be genetically modified to express an orthogonal or heterologous gene in a glycosylation system (e.g., an N-linked glycosylation system and/or an O-linked glycosylation system). The N-linked glycosylation system of Campylobacter jejuni has been transferred to E. coli. (See Wacker et al., “N-linked glycosylation in Campylobacter jejuni and its functional transfer into E. coli,” Science 2002 Nov. 29; 298 (5599): 1790-3, the content of which is incorporated herein by reference in its entirety). In particular, the modified strain may be modified to express one or more genes of the pgl locus of C. jejuni or one or more genes of a homologous pgl locus. The genes of the pgl locus include pglG, pglF, pglE, wlaJ, pglD, pglC, pglA, pglB, pglJ, pglI, pglH, pglK, and gne, and are used to synthesize lipid-linked oligosaccharides (LLOs) and transfer the oligosaccharide moieties of the LLOs to a protein via an oligosaccharyltransferase.

In some embodiments, orthogonal or heterologous oligosaccharyltransferases (OST) which may be expressed in the genetically modified strains may include Campylobacter jejuni oligosaccharyltransferase PglB. The gene for the C. jejuni OST is referred to as pglB. PglB catalyzes transfer of an oligosaccharide to a D/E-Y-N-X-S/T motif (Y, X≠P) present on a protein.

Crude cell lysates may be prepared from the modified strains disclosed herein. The crude cell lysates may be prepared from different modified strains as disclosed herein and the crude cell lysates may be combined to prepare a mixed crude cell lysate. In some embodiments, one or more crude cell lysates may be prepared from one or more modified strains including a genomic modification (e.g., deletions of genes rendering the genes inoperable) that preferably result in lysates comprising sugar precursors for glycosylation at relatively high concentrations (e.g., in comparison to a strain not having the genomic modification). In some embodiments, one or more crude cell lysates may be prepared from one or more modified strains that have been modified to express one or more orthogonal or heterologous genes or gene clusters that are associated with glycoprotein synthesis. Preferably, the crude cell lysates or mixed crude cell lysates are enriched in glycosylation components, such as lipid-linked oligosaccharides (LLOs), glycosyltransferases (GTs), oligosaccharyltransferases (OSTs), or any combination thereof. More preferably, the crude cell lysates or mixed crude cell lysates are enriched in Man₃GlcNAc₂ LLOs representing the core eukaryotic glycan and/or Man₃GlcNAc₄Gal₂Neu₅Ac₂ LLOs representing the fully sialylated human glycan.

The disclosed crude cell lysates may be used in cell-free glycoprotein synthesis (CFGpS) systems to synthesize a variety of glycoproteins. The glycoproteins synthesized in the CFGpS systems may include prokaryotic glycoproteins and eukaryotic proteins, including human proteins. The CFGPS systems may be utilized in methods for synthesizing glycoproteins in vitro by performing the following steps using the crude cell lysates or mixtures of crude cell lysates disclosed herein: (a) performing cell-free transcription of a gene for a target glycoprotein; (b) performing cell-free translation; and (c) performing cell-free glycosylation. The methods may be performed in a single vessel or multiple vessels. Preferably, the steps of the synthesis method may be performed using a single reaction vessel. The disclosed methods may be used to synthesis a variety of glycoproteins, including prokaryotic glycoproteins and eukaryotic glycoproteins. The disclosed glycoproteins may be utilized in a variety of applications including vaccines and immunological compositions. The vaccines and immunological compositions including the disclosed glycoproteins may be lyophilized to extend their shelf life.

In various aspects, the systems and methods disclosed herein can incorporate non-canonical sugars and/or construct azido-sialoglycoprotein structures. In various aspects, one or more genetic constructs can be present in the CFPS and/or in vitro glycosylation assembly (IVA) reaction for expressing glycosyltransferases. In one or more aspects, the glycosyltransferases can include one or more of ApNGT, LgtB, CSTI, PdST6, or NeuA. In certain aspects, the ApNGT can be from, or derived from, Actinobacillus pleuropneumoniae, the CSTI can be from, or derived from, Campylobacter jejuni, the PAST6 can be from, or derived from, Photobacterium damselae.

In various aspects, the CFPS or in vitro glycosylation assembly (IVA) can be supplemented with sugar donors, including non-canonical sugars. In various aspects, the non-canonical sugars can include, but are not limited to C5 azido-sialic acid, C9-azido sialic acid, or both.

In the same or alternative aspects, the systems and methods disclosed herein can conjugate different or various species to a protein on an entity separate from the protein itself. In one aspect, this can occur via conjugation to an azido group. In certain aspects, the azido group can be provided from an azido-sialic acid, such as those discussed above. Any type of species or can be utilized for conjugation to the azido group. In one aspect, an alkyne-containing moiety is conjugated to the azido group. In aspects, the alkyne-containing moiety can be any type of alkyne-containing moiety. In various aspects, the alkyne-containing moiety can be a standard alkyne, a strained alkyne (e.g., Dibenzocyclooctyne (DBCO), a fluorophore, a chemical group, therapeutic agent, a chemical warhead, a nucleotide sequence, a protein, a carbohydrate, or a combination thereof.

In various aspects, any proteins can be utilized in the IVA. In various aspects, the proteins can be glycosylation site-containing proteins. In one or more aspects, the proteins and/or glycosylation site-containing proteins can be provided in the CFPS and/or IVA as a polynucleotide construct for expression therein and then can be utilized in the IVA. In various aspects, the glycosylation site-containing proteins can include an amino acid sequence recognized by ApNGT for glycosylation, otherwise known as a Glyctag (GGNWTT (SEQ ID NO: 18)). In various aspects, the glycosylation site can be present within the protein of interest and/or can be present on a fusion protein or linker associated with the protein of interest. In one or more aspects, the glycosylation site-containing protein can be an allergen protein, a model protein, a biological protein of interest, a therapeutic protein, a synthetic protein, an antibody or fragment thereof, a carrier protein with useful drug and/or therapeutic characteristics. In various aspects, example allergen proteins include Der p 1 (dust mite); Der p 2 (dust mite); Bos d 5 (milk); Bos d 4 (milk); Gal d 1 (ovomucoid); Gal d 2 (ovalbumin); Gal d 4 (lysozyme); Ara h 2 (peanut); Fel d 1 (cat); Amb a 1 (ragweed); Bet v 1 (birch pollen); Phl p 5 (grass pollen); Bla g 2 (cockroach); Cry j 1 (japanese cedar pollen). In various aspects, the proteins for use in the IVA, methods, and/or systems disclosed herein can include an amino acid sequence encoded in a polynucleotide that is at least about 50%, at least about 60%, at least about 70%, at least about 80%, at least about 85%, at least about 90%, at least about 95%, or at least about 99% identical to one or more of SEQ ID NOs: 1-14.

### ILLUSTRATIVE EMBODIMENTS

The following embodiments are illustrative and should not be interpreted to limit the scope of the claimed subject matter.
1. Incorporation of non-canonical sugars into glycoprotein structures using a cell free system: This method comprises one or more of the following:
   1.1 DNA design of genetic constructs encoding proteins containing a recognizable amino acid sequence that comprises a glycosylation site;
   1.2 DNA design of genetic constructs encoding bacterial glycosyltransferases with purification tags that retain activity;
   1.3 Expression of glycosylation site-containing proteins in genetically modified E. coli extracts;
   1.4 Expression of glycosyltransferase enzymes in genetically modified E. coli extracts;
   1.5 Assembly of expressed glycosyltransferase enzymes and glycosylation site-containing proteins in in vitro glycosylation reactions;
   1.6 Supplementation of sugar donors, including non-canonical sugars C5 azido-sialic acid or C9-azido sialic acid, in in vitro glycosylation reactions;
   1.7 Optimization of cell free protein synthesis expression conditions;
   1.8 Optimization of in vitro glycosylation reaction conditions;
   1.9 Evaluation of installed glycan by mass spectrometry;
   1.10 Evaluation of glycan binding by lectin using an AlphaLISA assay.
2. Conjugation of other moieties through non-canonical sugar: This method comprises:
   2.1 Assembly of click chemistry reagents to conjugate an alkyne-containing moiety to the azide-containing sugar.
3. The method of embodiment 1 where the glycosylated protein is an allergen including but not limited to the following: Der p 1 (dust mite); Der p 2 (dust mite); Bos d 5 (milk); Bos d 4 (milk); Gal d 1 (ovomucoid); Gal d 2 (ovalbumin); Gal d 4 (lysozyme); Ara h 2 (peanut); Fel d 1 (cat); Amb a 1 (ragweed); Bet v 1 (birch pollen); Phl p 5 (grass pollen); Bla g 2 (cockroach); Cry j 1 (japanese cedar pollen), or combinations thereof.
4. The method of embodiment 1 where the glycosylated protein is a model protein.
5. The method of embodiment 1 where the glycosylated protein is a biological protein of interest.
6. The method of embodiment 1 where the glycosylated protein is a synthetic protein.
7. The method of embodiment 1 where the glycosylated protein is an antibody.
8. The method of embodiment 1 where the glycosylated protein is a carrier protein with useful drug characteristics.
9. The method of embodiment 1 where the protein and glycosylation site are separated by a fusion protein.
10. The method of embodiment 1 where the protein and glycosylation site are separated by a linker.
11. The method of embodiment 1 where cell-free extracts are derived from other organisms.
12. The method of embodiment 1 where the azide-group is at a different position on the sialic acid.
13. The method of embodiment 1 where glycan binding to lectin is assessed by biolayer interferometry.
14. The method of embodiment 1 where glycan binding to lectin is assessed by surface plasmon resonance.
15. The method of embodiment 2 where the alkyne is a standard alkyne.
16. The method of embodiment 2 where the alkyne is a strained alkyne (DBCO).
17. The method of embodiment 2 where the alkyne-containing moiety is a fluorophore.
18. The method of embodiment 2 where the alkyne-containing moiety is a chemical group.
19. The method of embodiment 2 where the alkyne-containing moiety is a chemical warhead.
20. The method of embodiment 2 where the alkyne-containing moiety is a nucleotide sequence.
21. The method of embodiment 2 where the alkyne-containing moiety is a protein.
22. The method of embodiment 2 where the alkyne-containing moiety is a carbohydrate.

### Additional Embodiments

1. A method for preparing an azido-sialoglycoprotein, the method comprising: in a cell-free in vitro glycosylation assembly (IVA) reaction, providing a polypeptide comprising an amino acid sequence recognized by ApNGT for glycosylation; wherein the IVA comprises one or more of: ApNGT, LgtB, CSTI, PdST6, NeuA, or CTP.
2. The method of additional embodiment 1, wherein the IVA comprises one or more E. coli lysates.
3. The method of additional embodiment 2, wherein at least one of the E. coli lysates is derived from a genetically modified E. coli strain.
4. The method of additional embodiment 2, wherein the one or more E. coli lysates expresses one or more of ApNGT, LgtB, CSTI, PdST6, or NeuA.
5. The method of any of additional embodiments 1-4, wherein the IVA comprises one or sugars comprising an azido moiety.
6. The method of additional embodiment 5, wherein the one or more sugars comprising an azido moiety is C5 azido-sialic acid, C9-azido sialic acid, or both.
7. The method of any of additional embodiments 1-6, wherein the polypeptide is expressed in a cell-free protein synthesis system (CFPS).
8. The method of any of additional embodiments 1-7, wherein the amino acid sequence recognized by ApNGT for glycosylation is GGNWTT (SEQ ID NO: 18).
9. The method of any of additional embodiments 1-8, wherein the amino acid sequence recognized by ApNGT for glycosylation is attached to the polypeptide by a linker or fusion protein.
10. The method of any of additional embodiments 1-9, wherein the polypeptide is an allergen protein, a synthetic protein, or an antibody or fragment thereof.
11. The method of any of additional embodiments 1-10, wherein the polypeptide is an allergen protein, and wherein the allergen protein is one or more of Der p 1; Der p 2; Bos d 5; Bos d 4; Gal d 1; Gal d 2; Gal d 4; Ara h 2; Fel d 1; Amb a 1; Bet v 1; Phl p 5; Bla g 2; or Cry j 1.
12. The method of any of additional embodiments 1-10, further comprising converting the polypeptide into an azido-sialoglycoprotein.
13. The method of additional embodiment 12, conjugating an alkyne-containing moiety to the azido-sialoglycoprotein.
14. A composition comprising the IVA of additional embodiment 1.
15. The composition of additional embodiment 14, further comprising one or sugars comprising an azido moiety.
16. The composition of additional embodiment 15, wherein the one or more sugars comprising an azido moiety is C5 azido-sialic acid, C9-azido sialic acid, or both.
17. A genetically engineered E. coli strain comprising one or more nucleic acid sequences encoding one or more of: ApNGT, LgtB, CSTI, PAST6, or NeuA.
18. The genetically engineered E. coli strain of additional embodiment 17, wherein the ApNGT is from Actinobacillus pleuropneumoniae, wherein the LgtB is from Neisseria gonorrhoeae, wherein the CSTI is from Campylobacter jejuni, and wherein the PAST6 is from Photobacterium damselae.
19. A kit comprising the IVA of additional embodiment 1.
20. The kit of additional embodiment 19, further comprising one or more E. coli lysates, one or sugars comprising an azido moiety, or both.

### EXAMPLES

The following Examples are illustrative and are not intended to limit the scope of the claimed subject matter.

### Example 1. GlycoCAP: A Cell-Free, Bacterial Glycosylation Platform for Building Clickable Azido-Sialoglycoproteins

Glycan-binding receptors known as lectins represent a class of potential therapeutic targets. Yet, the therapeutic potential of targeting lectins remains largely untapped due in part to limitations in tools for building glycan-based drugs. One group of desirable structures is proteins with noncanonical glycans. Cell-free protein synthesis systems have matured as a promising approach for making glycoproteins that may overcome current limitations and enable new glycoprotein medicines. Yet, this approach has not been applied to the construction of proteins with noncanonical glycans. To address this limitation, we developed a cell-free glycoprotein synthesis platform for building noncanonical glycans and, specifically, clickable azido-sialoglycoproteins (called GlycoCAP). The GlycoCAP platform uses an Escherichia coli-based cell-free protein synthesis system for the site-specific installation of noncanonical glycans onto proteins with a high degree of homogeneity and efficiency. As a model, we construct four noncanonical glycans onto a dust mite allergen (Der p 2): α2,3 C5-azido-sialyllactose, α2,3 C9-azido-sialyllactose, α2,6 C5-azido-sialyllactose, and α2,6 C9-azido-sialyllactose. Through a series of optimizations, we achieve more than 60% sialylation efficiency with a noncanonical azido-sialic acid. We then show that the azide click handle can be conjugated with a model fluorophore using both strain-promoted and copper-catalyzed click chemistry. We anticipate that GlycoCAP will facilitate the development and discovery of glycan-based drugs by granting access to a wider variety of possible noncanonical glycan structures and also provide an approach for functionalizing glycoproteins by click chemistry conjugation.

### INTRODUCTION

Lectins are glycan-binding receptors that represent a promising class of therapeutic targets. Sialic acid-binding immunoglobu-lin-like lectins (Siglecs) are one example of a lectin family that has shown particular promise as targets in treating allergic inflammation and preventing anaphylaxis (1-14). Siglecs are transmembrane receptors that bind sialic acid-containing glycans and are differentially expressed on immune cells suchthat each immune cell has its own Siglec expression profile (15-19). Most, but not all, Siglecs have immune suppressive activity by virtue of possessing cytoplasmic immunoreceptor tyrosine-based inhibitory motifs that recruit phosphatases (19). Therefore, Siglecs not only offer an avenue for immune cell inhibition but also provide an approach for cell-specific targeting.

Despite their promise, the full potential of lectin-targeting therapeutics, like Siglecs, remains untapped. This is because lectins specifically bind glycans, and we lack adequate tools to rapidly build and manufacture a multiplicity of structures needed for biochemical, mechanistic, and structural studies to create glycan-based, lectin-binding drugs. Current approaches often utilize mammalian cell culture, which yields heterogeneous glycan populations, (20-23) or chemoenzymatic synthesis, which produces homogeneous glycan populations but can be expensive and difficult to scale up (5,7,11,12,24-26).

Cell-free protein synthesis (CFPS) systems from Escherichia coli-based extracts have emerged as an approach to rapidly make glycoproteins (27-29) and could overcome the challenges of current manufacturing methods. Making glycoproteins in CFPS systems involves expressing glycosyltransferases from other organisms and supplementing the reaction with appropriate sugar substrates (e.g., UDP-glucose, lipid-linked oligosaccharides) to install glycans onto proteins. Cell-free glycoprotein synthesis systems have several advantages over conventional cellular manufacturing strategies. First, because E. coli lacks endogenous glycosylation machinery, site-specific glycosylation occurs without competition from other host glycosyltransferases. Second, cell-free systems are not limited by viability requirements, thus avoiding constraints arising from noncanonical substrates (30). Third, the lack of a cell membrane circumvents the need for membrane transport of provided sugar substrates. Finally, cell-free systems are amenable to scale-up for manufacturing, having demonstrated linear scalability over a six order of magnitude range in volume to reach the 100 L milestone (31,32).

To date, cell-free glycoprotein synthesis systems have been developed for making human and bacterial glycoproteins, (33-42) designing protein glycosylation sites, (43,44) and modularly constructing glycosylation pathways (45). However, while cell-free systems offer promise for making glycoproteins, there is one group of glycan structures that has shown therapeutic promise but has, to our knowledge, never been made in an integrated cell-free system: that group is noncanonical glycans.

Noncanonical glycans and, more specifically, clickable azido-sialoglycans have been used to optimize lectin-binding pocket interactions by chemically modifying sialic acid-containing glycans through click chemistry conjugation of small chemical groups (46-48). This approach has shown great promise in targeting Siglecs to treat allergy and autoimmune disease (5, 11-14, 49-52). Clickable azido-sialoglycans also offer opportunities for creating dual function therapeutics, functionalizing glycoprotein with favorable properties, and facilitating scientific research by providing methods for labeling and localizing proteins of interest (53-55).

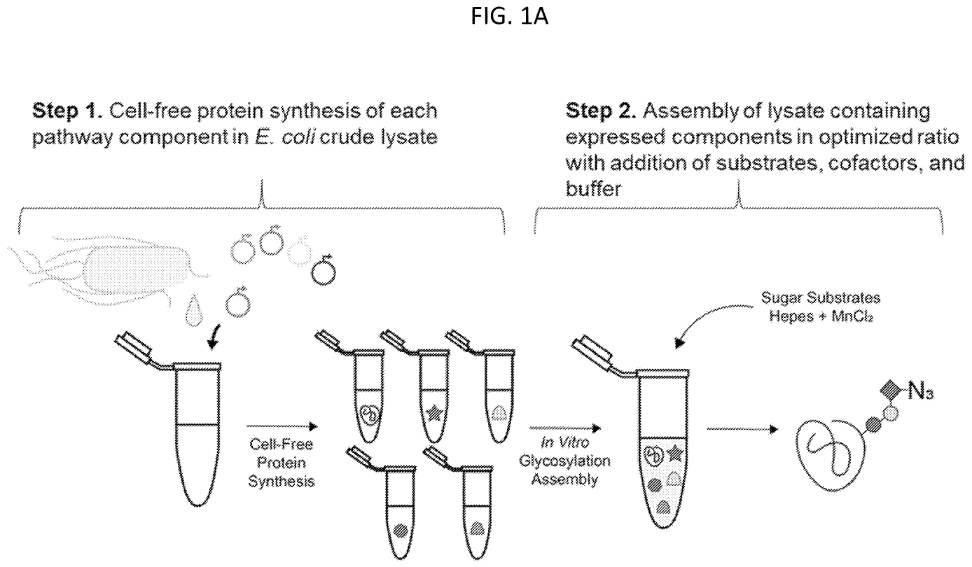

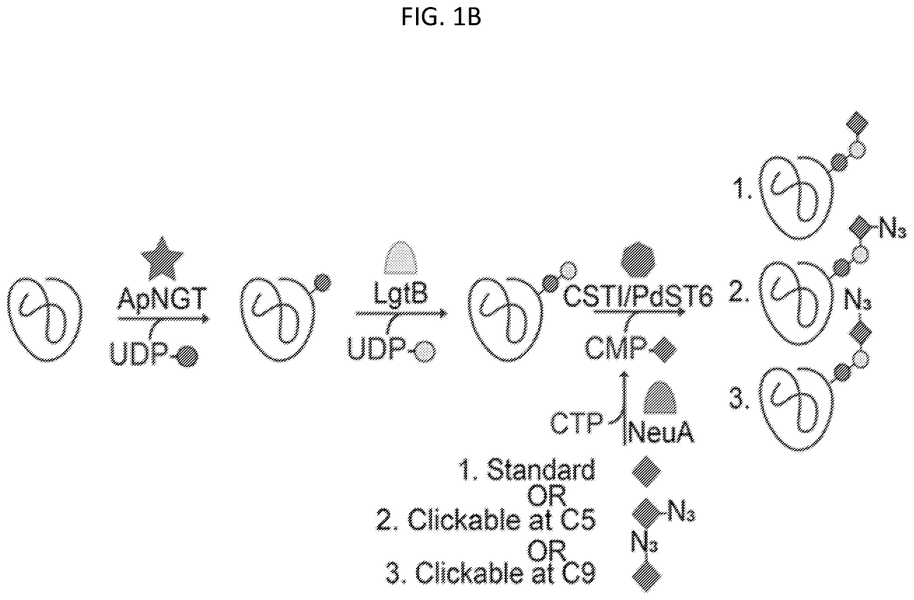

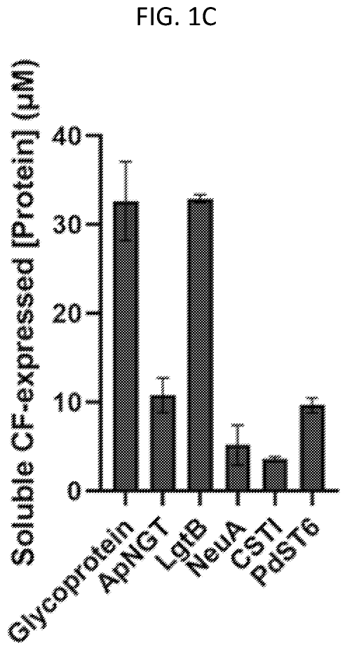

Here, we set out to leverage the advantages of a cell-free system to harness the promise of noncanonical glycans. Toward this aim, we developed a bacterial-based, cell-free glycosylation platform that enables site-specific installation of clickable azido-sialoglycans onto proteins (called GlycoCAP) (FIG. 1A). The key idea was to create cell-free biosynthesis “units” made from crude cell lysates that are selectively enriched with pathway enzymes expressed directly in lysates by CFPS. Then, these units are assembled modularly, in a mix-and-match fashion, to build defined glycosylation pathways harboring noncanonical glycans (FIG. 1B). As proof-of-concept, we apply GlycoCAP for the synthesis of four Siglec ligands, which are amenable to strain-promoted and copper-catalyzed click chemistry modification. The GlycoCAP platform contributes a set of tools for constructing noncanonical glycans onto proteins, which could open opportunities for therapeutically targeting Siglecs, among other lectins, and offers strategies for protein labeling, development of dual function therapeutics, and protein localization.

### Results

GlycoCAP Platform. The goal of this work was to create a bacterial-based, cell-free platform for installing noncanonical glycans onto desired target glycoproteins. Specifically, we focused on site-specific installation of azido-sialyllactose onto proteins as the azide handle on the glycan grants an avenue for conjugation by click chemistry for Siglec therapeutics. To achieve our goal, we (i) established a base pathway for installing sialyllactose onto proteins, (ii) implemented and optimized a pathway required for activating and incorporating azido-glycan substrates, and (iii) applied our pathway to produce azide-containing glycoproteins that could be modified through bioorthogonal chemistry.

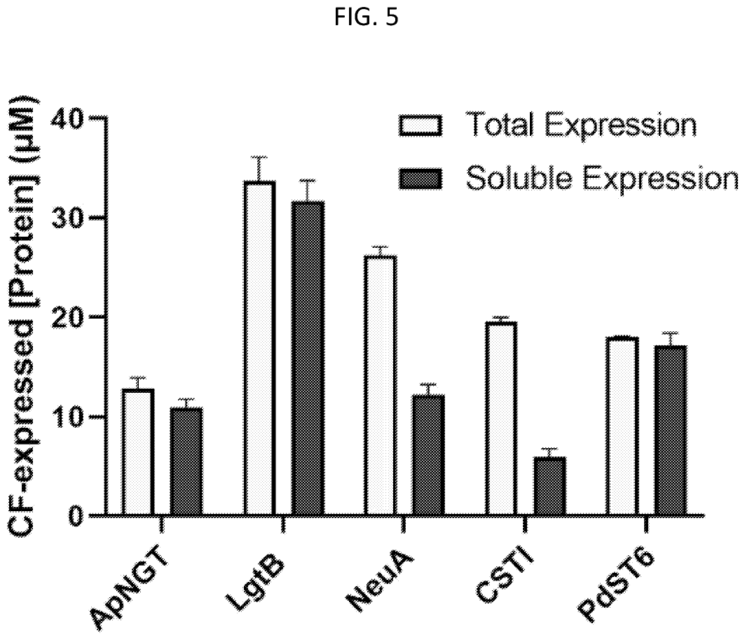

Establishing Conditions for Installing Sialyllactose. Initially, we set out to construct a minimal trisaccharide structure terminating in sialic acid on a model protein target. This involved two steps. First, we individually expressed multiple components of a bacterial glycosylation pathway we previously established (45) by CFPS in the lysate of an engineered E. coli strain (56). Pathway components include (i) the target glycoprotein, which has been designed with an amino acid sequence (43) recognized for N-linked glycosylation (Table 1 below), (ii) ApNGT, which is a glycosyltransferase from Actinobacillus pleuropneumoniae and installs glucose onto an asparagine residue, (57, 58) (iii), LgtB, which is from Neisseria gonorrhoeae and elaborates a galactose onto the glucose in a β1,4 linkage, and (iv) CSTI, from Campylobacter jejuni, which elaborates a sialic acid onto the lactose in an α2,3 linkage, or PdST6, from Photobacterium damselae, which elaborates a sialic acid onto the lactose in an α2,6 linkage. Soluble expression and yield of pathway components were quantified by [14C]-leucine incorporation (FIGS. 1C and 5).

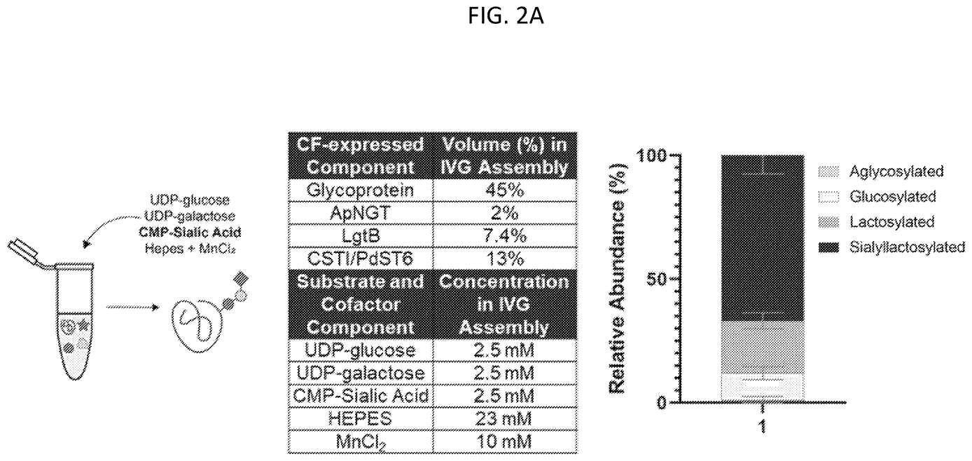

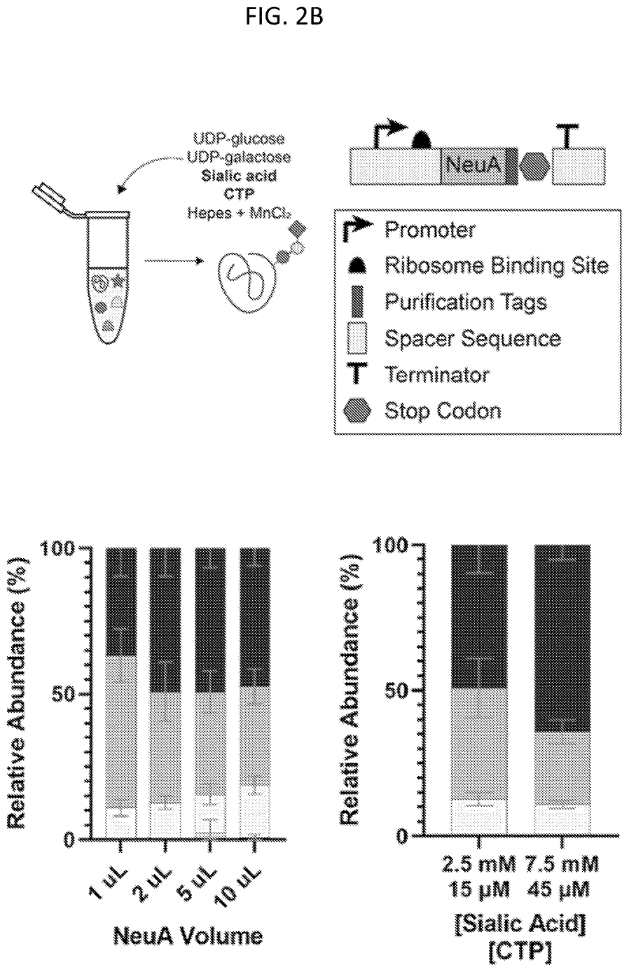

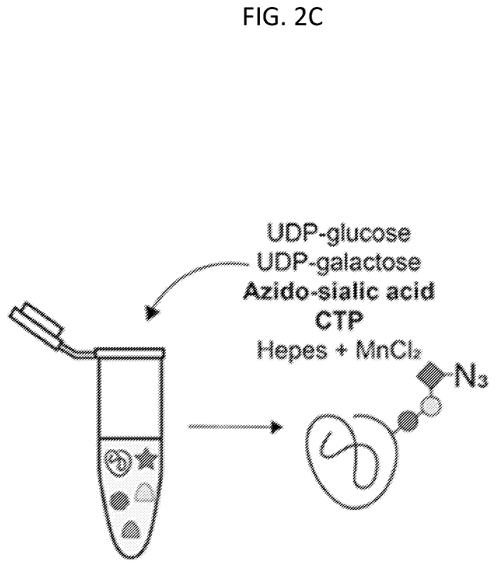

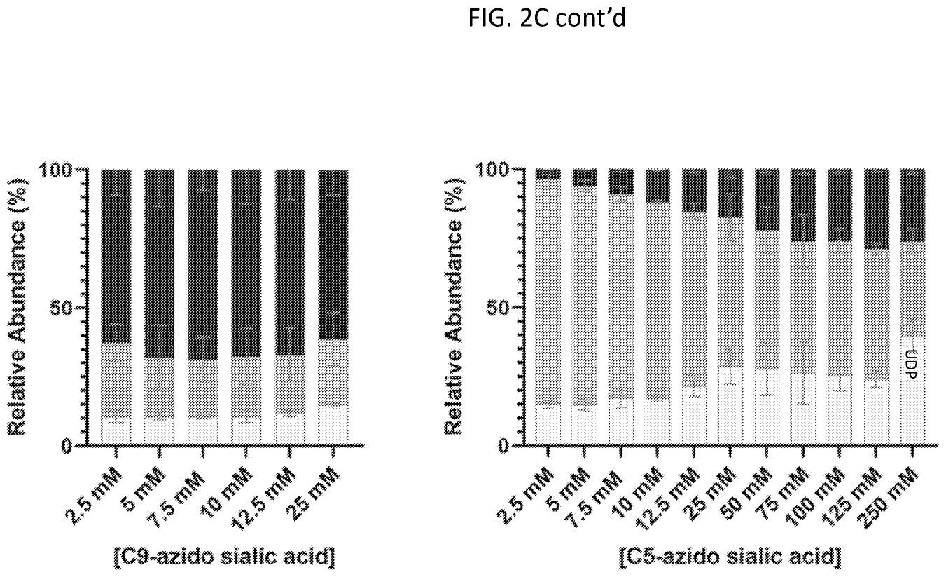

In step 2, following the expression of pathway components, we assembled in vitro glycosylation (IVA) reactions such that the reactions were composed of an optimized percent volume of each CF-expressed component and optimized molar concentrations of substrates and cofactors based on parameters previously published. 45 IVA reactions included CF-expressed (i) target glycoprotein (45% by volume), (ii) ApNGT (2%), (iii) LgtB (7.4%), and (iv) CSTI (13%) or PdST6 (13%) along with (v) activated sugar donors UDP-glucose (2.5 mM) UDP-galactose (2.5 mM), and CMP-sialic acid (2.5 mM) and MnCl2 (10 mM) and HEPES (23 mM) buffer. As a model protein, we used dust mite allergen (Der p 2) for glycan installation (59, 60). IVA reactions were incubated overnight, re-supplemented with sugar substrate, and incubated for an additional 24 h. Glycosylated allergen was isolated by affinity-tag purification, and liquid chromatography—mass spectrometry (LC-MS) was used to calculate the glycosylation efficiency. We determined a base case standard for glycosylation efficiency, which we determined to be 67±7% (FIG. 2A).

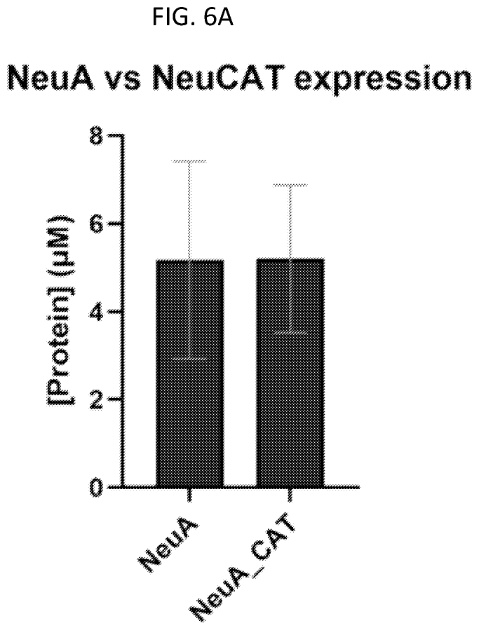

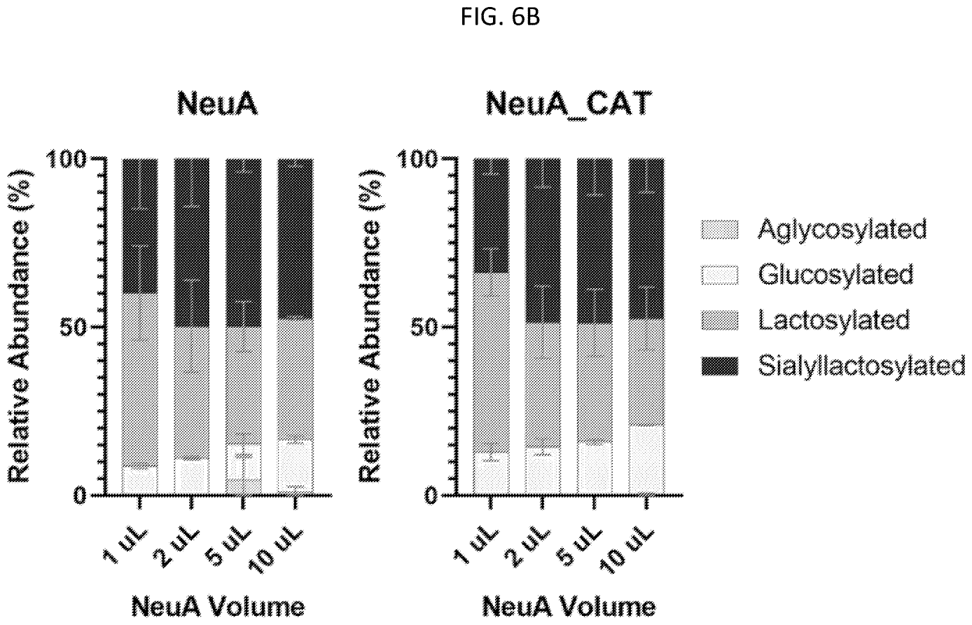

Extending the Pathway for Incorporation of Non-activated Sialic Acids. With a base case glycoprotein synthesis system in place, we turned our efforts toward incorporating noncanonical glycans. Since noncanonical sialic acids are only available in their nonactivated form (not conjugated to a phosphonucleotide), incorporation of a noncanonical sialic acid requires the addition of N-acylneuraminate cytidylyltransferase (NeuA) (FIGS. 6A and 6B and Table 2 below), which catalyzes the formation of CMP-sialic acid from canonical or noncanonical sialic acid and cytidine triphosphate (CTP) to produce the appropriate, activated sugar donor for CSTI/PdST6 enzymes. We performed a titration of CFPS-expressed NeuA into a series of IVA reactions where 2.5 mM standard sialic acid and 15 μM CTP were supplemented. NeuA was added at a volume of 1, 2, 5, or 10 μL. Adding 2 μL (corresponding to 4.5% of the reaction volume) was enough to achieve the optimal glycosylation efficiency of 49±10%, meaning the relative abundance of the final desired sialylated product is ~49% and the remaining species are earlier pathway intermediates (FIG. 2B).

To determine whether the lower efficiency compared to the base case standard is a result of the IVA assembly being substrate limited, we titrated additional sialic acid and CTP. Increasing [sialic acid] to 7.5 mM and [CTP] to 45 μM achieved a glycosylation efficiency of 64±5%, which is on par with the base case standard using the activated sialic CMP-acid (FIG. 2B).

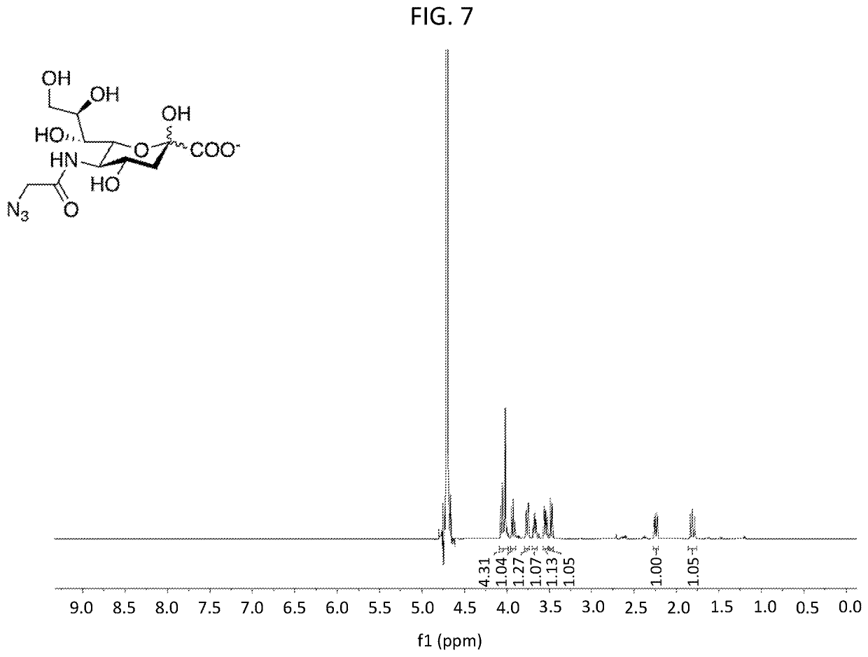

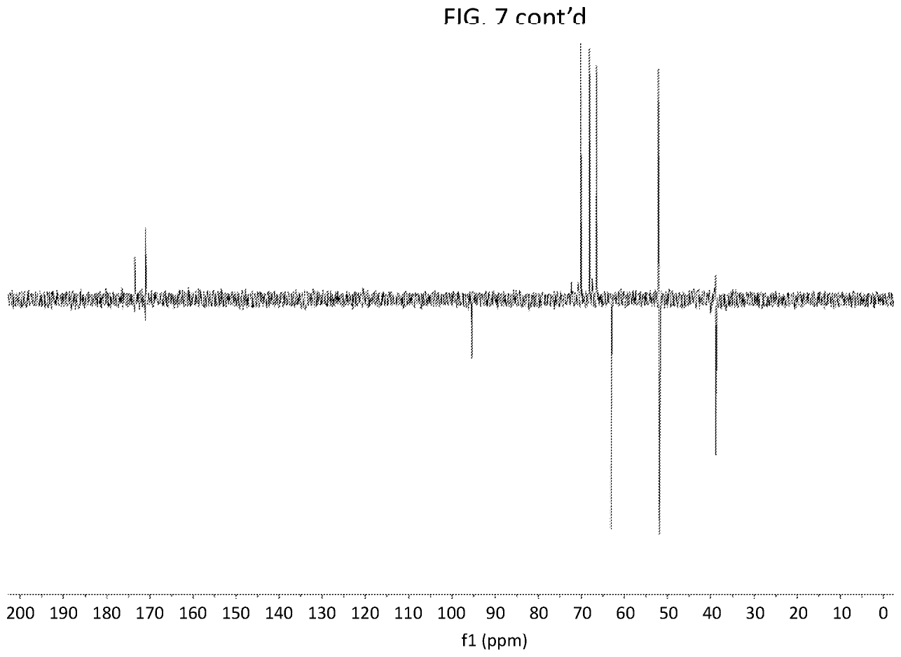

Incorporation of C9- and C5-Azido-Sialic Acids. We next performed a titration of azido-sialic acids where the azide group is either at the 9th carbon of sialic acid (C9-azido-sialic acid) or where an azidoacetyl group is on the 5th carbon of sialic acid (C5-azido-sialic acid) (FIG. 7). The goal was to test whether the enzymes of our pathway are permissive to an additional chemical group on their sialic acid substrate. C9-azido-sialic acid was incorporated at an equal or better efficiency than canonical sialic acid, where 7.5 mM C9-azido-sialic acid and 15 μM CTP led to 69±8% glycosylation efficiency (FIG. 2C). C5-azido-sialic acid could also be incorporated. However, higher concentrations of C5-azido-sialic acid were required and still did not match the base case efficiency with the canonical sialic acid (FIG. 2A), indicating that the pathway enzymes are less permissive to C5-azido-sialic acid than to C9-azido-sialic acid, possibly due to disrupted binding pocket interactions caused by the azide group at the 5th carbon position. By adding 125 mM C5-azido-sialic acid and 750 μM CTP, we were able to achieve a glycosylation efficiency of 29±1% (FIG. 2C).

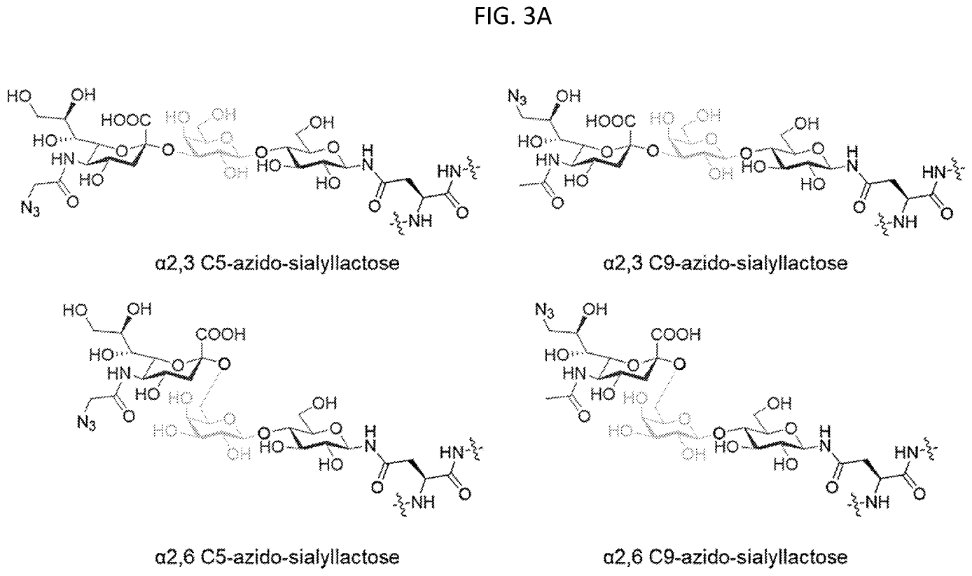

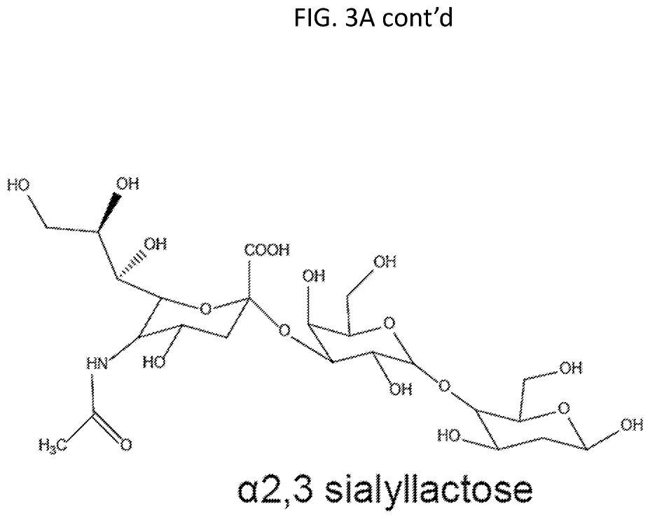

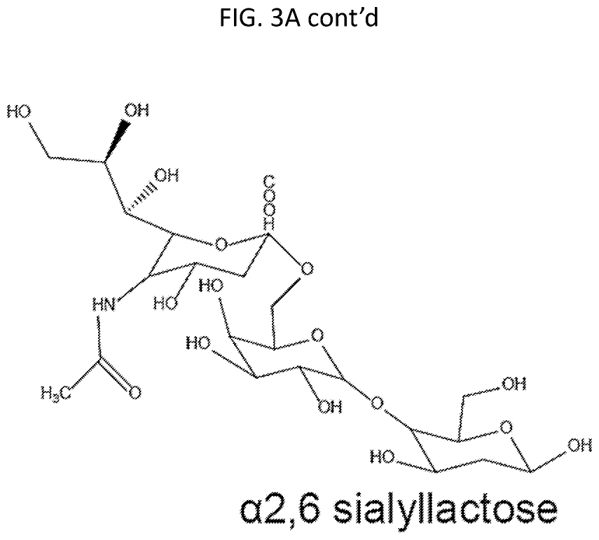

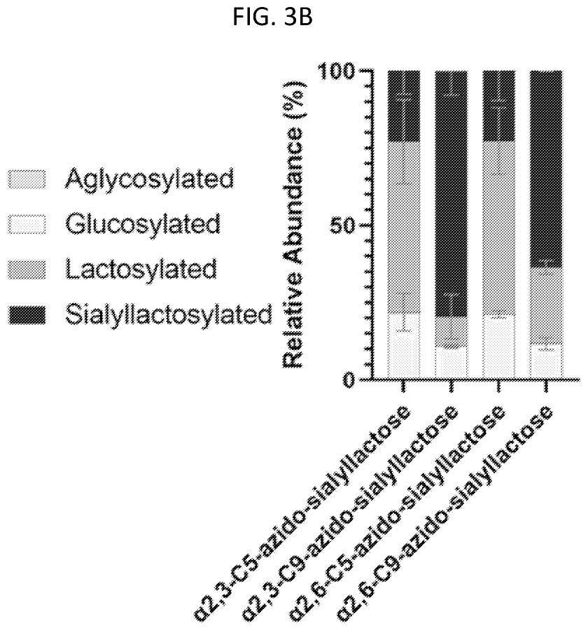

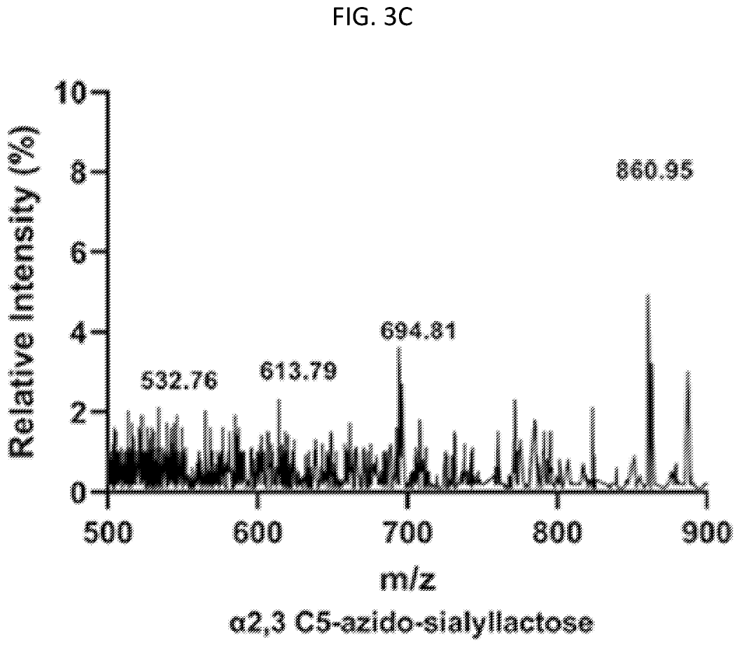

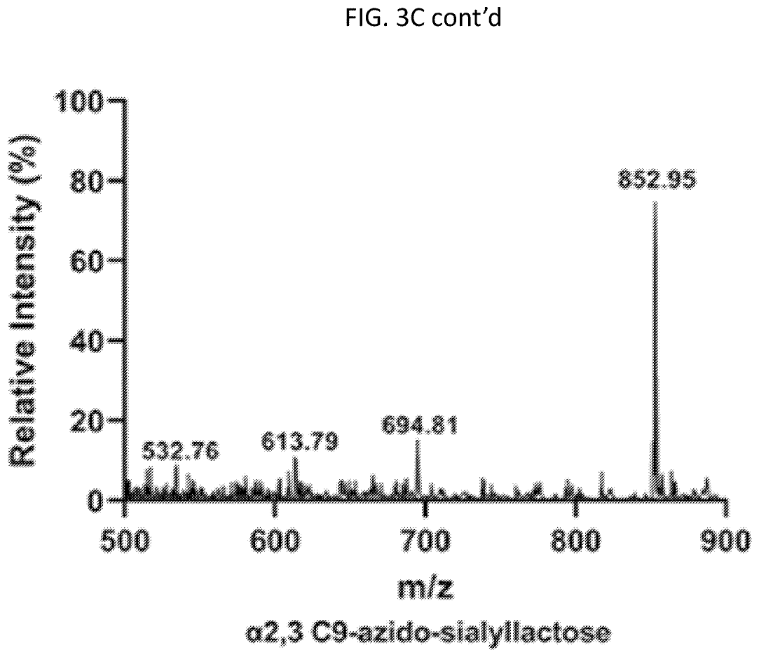

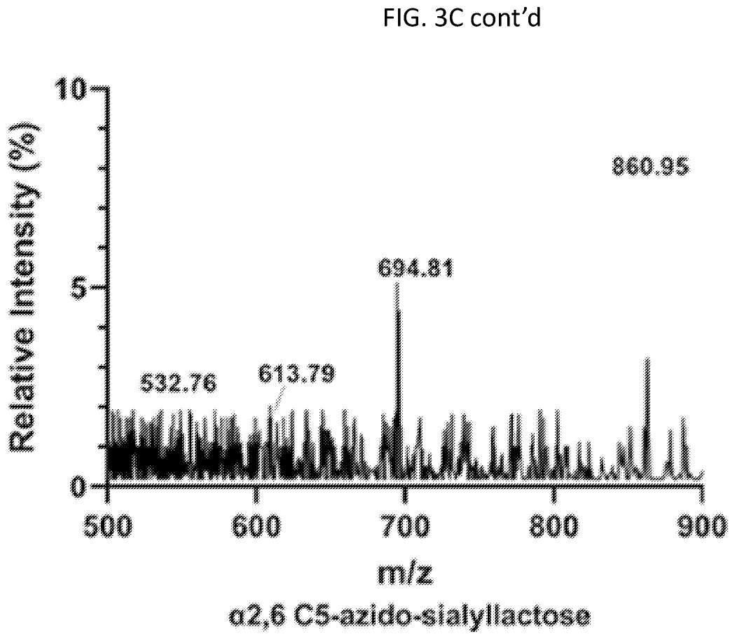

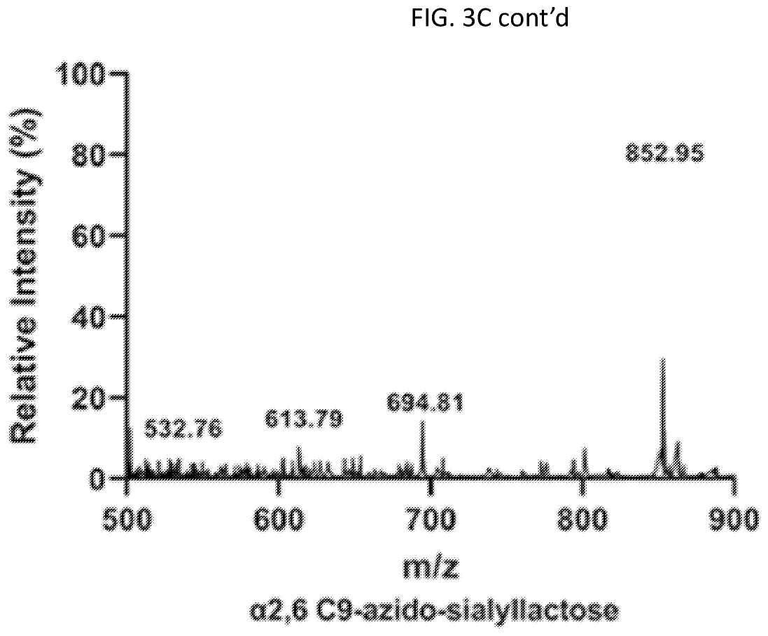

GlycoCAP Production of α2,3-C5-Azido-sialyllactose, α2,3-C9-Azido-sialyllactose, α2,6-C5-Azido-sialyllactose, and α2,6-C9-Azido-sialyllactose. With optimal conditions established for noncanonical azido-sialic acid incorporation, we installed four different clickable azido-sialoglycans onto proteins (FIG. 3A). Under optimized GlycoCAP conditions, α2,3-C5-azido-sialyllactose was produced at 23±7%, efficiency, α2,3-C9-azido-sialyllactose was produced at 80±8%, efficiency, α2,6-C5-azido-sialyllactose was produced at 23±10%, efficiency, and α2,6-C9-azido-sialyllactose was produced at 64±1% efficiency (FIG. 3B). MS confirmed the production of these species and demonstrated the relative proportion of reaction intermediates and the final product present in the final reaction (FIG. 3C).

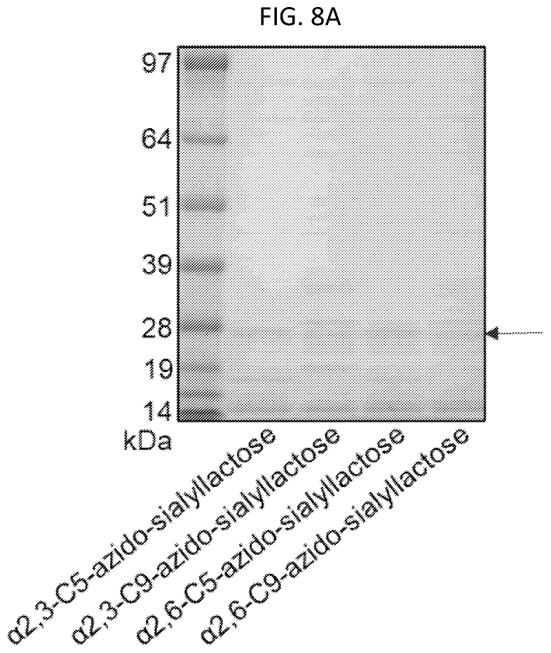

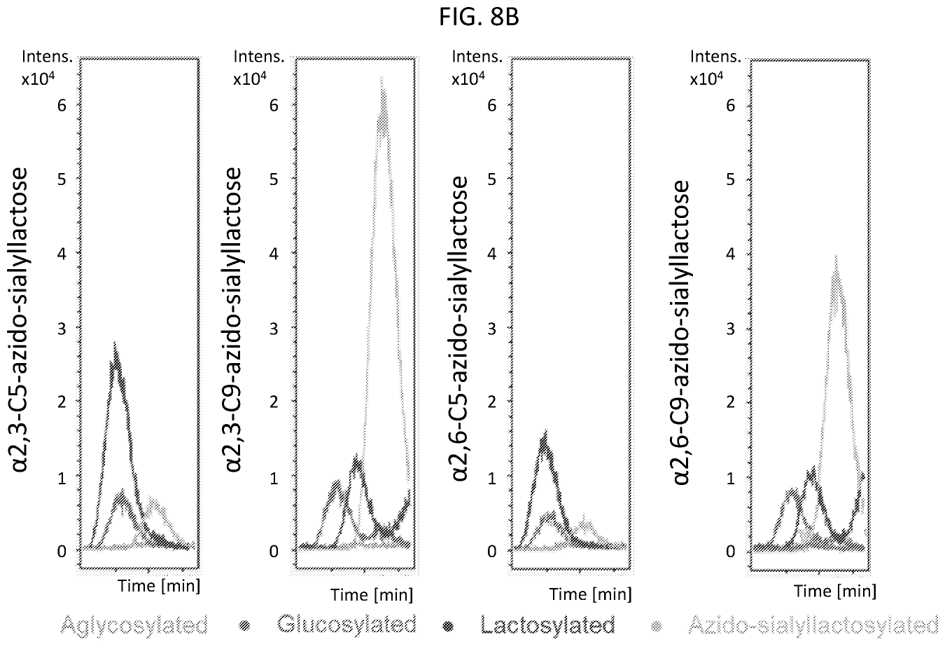

Preparative Scale Production of Azido-Sialoglyco-proteins. As a representative synthesis to demonstrate the scalability of the GlycoCAP platform, we performed 1 mL IVAs for each of the four clickable azido-sialoglycoproteins. After purification, we characterized the protein yield by UV absorbance at 280 nm. The abundance of azido-sialylated protein relative to other pathway intermediates was deter-mined by MS and applied as a percentage to the total protein yield to estimate the final product yield. Preparation at this scale yielded approximately 70 μg of C5-azido-α2,3-sialyllac-tose, 200 μg of C9-azido-α2,3-sialyllactose, 50 μg of C5-azido-α2,6-sialyllactose, and 150 μg of C5-azido-α2,3-sialyllactose (Table 1 and FIGS. 8A and 8B).

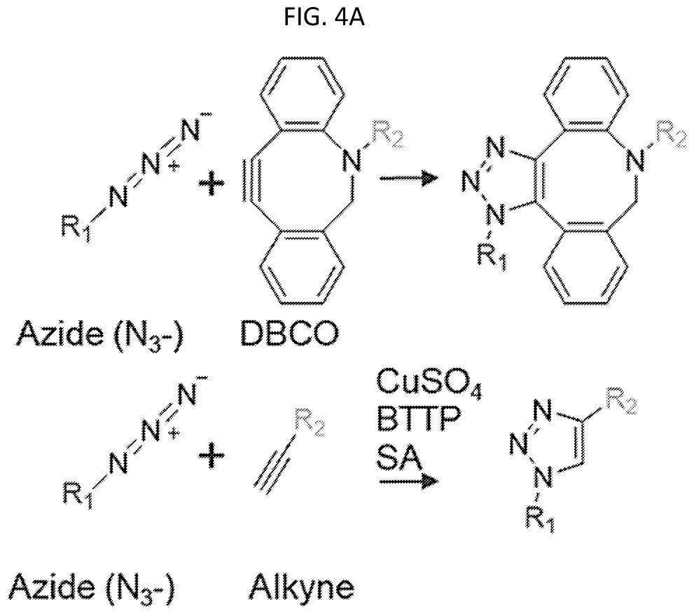

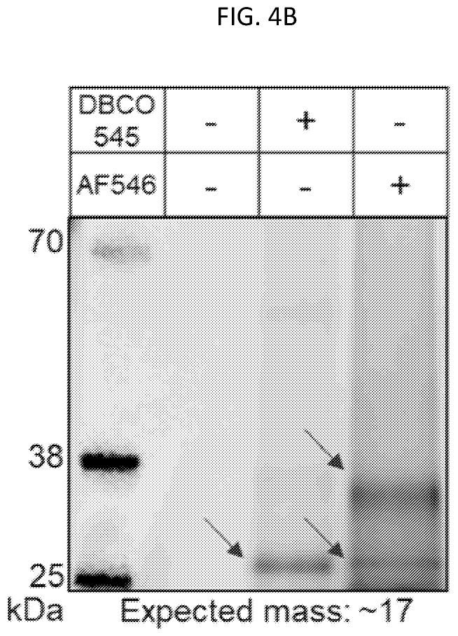

Click Chemistry Conjugation of Clickable Azido-Sialoglycoproteins. To show that installed azido-sialoglycans are amenable to click chemistry conjugation, we next performed both strain-promoted click chemistry with a fluorophore containing a dibenzocyclooctyne (DBCO) group (FIG. 4A, top) and copper-catalyzed click chemistry with a fluorophore containing a standard alkyne (FIG. 4A, bottom). In the absence of copper, steric strain around the alkyne is required for successful conjugation with DBCO. Copper-catalyzed reactions yield a smaller intervening triazole ring. Samples were run on a NuPAGE Bis-Tris gel and imaged under fluorescent excitation such that only fluorescent bands are visible. Click conjugation was successful with both methods as indicated by the presence of fluorescent bands at the expected masses for the monomer form of the Der p 2 glycoprotein (~17 kDa) and the dimer form (~34 kDa) (FIG. 4B). Additional weight from glycosylation and click modification in addition to incomplete denaturation of Der p 2 due to heat restrictions imposed by the fluorophore's heat sensitivity and the allergen's intrinsic stability likely influence its appearance on the gel slightly above its expected mass. The high intensity band at ~34 kDa for the copper-catalyzed click reaction indicates that these conditions favor dimer formation of the target glycoprotein.

### DISCUSSION

Here, we developed the GlycoCAP platform. GlycoCAP enables installation of noncanonical glycans onto proteins in a cell-free glycoprotein synthesis system. We demonstrated the utility of GlycoCAP by producing four noncanonical glycans: α2,3 C5-azido-sialyllactose, α2,3 C9-azido-sialyllactose, α2,6 C5-azido-sialyllactose, and α2,6 C9-azido-sialyllactose, all of which can be functionalized by click chemistry.

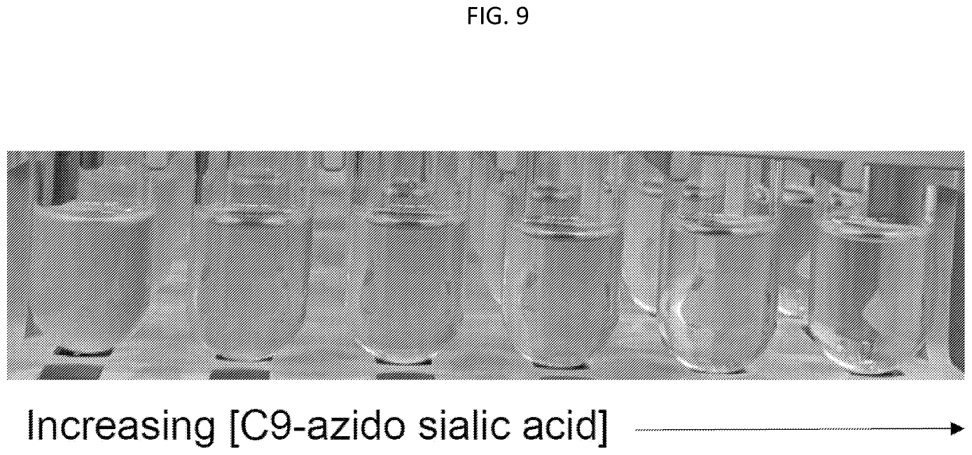

GlycoCAP offers several potential advantages for incorporating noncanonical glycans onto proteins. For example, freedom from constraints imposed by living cells may grant access to a wider repertoire of noncanonical glycans than is possible in living cells. As an example, we observed that culturing living E. coli with increasing [C9-azido-sialic acid] led to decreasing optical densities under otherwise equivalent culture conditions (FIG. 9). The observation that C9-azido-sialic acid incorporated as efficiently as standard sialic acid in our cell-free system provides proof of concept that our approach can overcome cell toxicity challenges and may be able to build structures otherwise inaccessible.

While GlycoCAP offers some benefits, there remains room for improvement. For example, yields of the desirable products were not 100%. To address this limitation and make the approach suitable for longer, more complex glycans, future efforts can seek to identify glycosyltransferases with higher efficiencies. Reduced permissivity toward C5-azido-sialic acid—possibly due to disrupted binding pocket interactions in NeuA caused by the position of the azide group—could be overcome by future efforts to engineer enzymes to alter promiscuity and efficiency. The GlycoCAP platform could be used to quickly screen a wide range of enzymes toward these efforts.

The GlycoCAP platform offers a range of potential applications. As an example, higher affinity glycan ligands for Siglec binding to generate novel allergy treatments could be pursued. Studies have shown that coengagement of a Siglec receptor with the high affinity IgE receptor (FcεRI) on the surface of mast cells results in robust inhibition of mast cell activation and anaphylaxis (5, 12). Siglec-3 (CD33) is expressed on the surface of mast cells and basophils and its endogenous ligand is α2,3 sialyllactose and α2,6 sialyllactose-both of which form the basis of the structures made here. GlycoCAP could be used to install clickable azido-sialoglycans onto allergen, which would engage allergen-specific IgE attached to FcεRI. The azido-sialoglycan could then be chemically modified by clicking on small chemical groups to improve CD33 binding pocket interactions and increase ligand affinity (49,51,52). This would generate a dual-specific therapeutic that co-engages IgE/FcεRI and CD33 to inhibit the allergic response. Siglec-8 has been targeted by liposomes decorated with click chemistry modified, sulfated sialoglycans to treat eosinophilic and mast cell diseases, (7) and Siglecs have been shown to play a role in neurodegenerative disease (61) and cancer (62, 63). The tools produced here could aid in the therapeutic development for a range of pathologies.

Looking forward, GlycoCAP provides a potentially powerful approach to discover, study, and optimize glycosylation pathways for noncanonical sugars. This could facilitate basic research and open the door for functionalizing glycoproteins, including conjugating a small-molecule drug to an antibody for localized delivery, modulating drug properties by conjugating property-endowing moieties, or barcoding or labeling proteins of interest with a nucleotide sequence or fluorophore. In addition, GlycoCAP could broaden capabilities for targeting lectins and Siglecs, paving the way for a novel class of medicines that are glycan-based therapeutics.

### Materials and Methods

Plasmid and DNA Design. The protein sequence of Der p 2 allergen was retrieved from Uniprot (accession ID: ALL2_DERPT), signaling sequence removed, and then codon optimized for E. coli K12 strains into a DNA sequence using the IDT Codon Optimization Tool. Additional synthetic sequences were added to optimize expression and to enable site-specific glycosylation and affinity purification (Table 1). This insert as well as the NeuA inserts (Table 2) were synthesized into a pJL1 backbone at NdeI and SalI restriction sites by Twist Biosciences.

TABLE 1
DNA Sequence of target glycoprotein
(in pJLI backbone)
Cat linker Protein BamHI linker Glyctag
His tag Stop codon

ATGGAGAAAAAAATCGATCAGGTGGATGTTAAAGATTGCG
CCAACCACGAAATCAAAAAAGTTCTGGTTCCGGGTTGTCA
TGGTAGCGAACCGTGTATTATTCATCGTGGTAAACCGTTT
CAGCTGGAAGCAGTTTTTGAAGCAAATCAGAATACCAAAA
CCGCCAAGATTGAAATCAAAGCCAGCATTGATGGTCTGGA
AGTTGATGTTCCGGGTATTGATCCGAATGCATGCCACTAT
ATGAAATGTCCGCTGGTTAAAGGTCAGCAGTATGATATCA
AATATACCTGGAACGTGCCGAAAATTGCACCGAAAAGCGA
AAATGTTGTTGTGACCGTTAAAGTGATGGGTGATGATGGT
GTTCTGGCATGTGCCATTGCAACCCATGCAAAAATTCGTG
ATGGATCCGGTCGTGCAACCACAGGTGGTAATTGGACAAC
CCGTGCCGGTGGTCATCACCATCATCACCATTAA
(SEQ ID NO: 17)

TABLE 2
NeuA DNA design with (top) and without
(bottom) a CAT linker
A CAT linker sequence (orange) was included
at the 5′ end of the NeuA sequence, which
serves to standardize the ribosome binding
site and can improve expression.

ATGGAGAAAAAAATCCGCACCAAAATTATCGCAATTATTCCGGCA
CGTAGCGGTAGCAAAGGTCTGCGTAACAAAAATGCACTGATGCTG
ATTGATAAACCGCTGCTGGCATATACCATTGAAGCAGCACTGCAG
AGCGAAATGTTTGAAAAAGTTATTGTTACCACCGACAGCGAACAG
TATGGTGCAATTGCCGAAAGCTATGGTGCAGATTTTCTGCTGCGT
CCGGAAGAACTGGCAACCGATAAAGCAAGCAGCTTTGAATTTATC
AAACATGCCCTGAGCATCTATACCGATTATGAAAATTTTGCACTG
CTGCAGCCGACCAGTCCGTTTCGTGATAGCACCCATATTATTGAA
GCCGTGAAACTGTATCAGACCCTGGAAAAGTATCAGTGTGTTGTT
AGCGTTACCCGTAGCAATAAACCGAGCCAGATTATTCGTCCGCTG
GATGATTATAGCACCCTGAGCTTTTTTGATCTGGACTACAGCAAA
TATAACCGCAACAGCATTGTTGAATATCATCCGAATGGTGCCATT
TTTATCGCCAACAAACAGCATTATCTGCATACCAAACATTTTTTC
GGTCGTTATAGCCTGGCCTACATCATGGATAAAGAAAGCTCACTG
GATATCGATGATCGCATGGATTTTGAACTGGCCATTACCATTCAG
CAGAAGAAAAATCGTCAGAAGATTCTGTACCAGAACATCCATAAT
CGCATCAACGAAAAACGCAACGAATTTGATAGCGTGAGCGATATT
ACCCTGATTGGTCATAGCCTGTTTGATTATTGGGACGTGAAAAAG
ATCAACGACATCGAAGTTAATAACCTGGGTATTGCCGGTATCAAC
AGCAAAGAATATTACGAGTACATCATCGAGAAAGAGCGCATTGTT
AATTTTGGCGAATTCGTGTTTATCTTTTTCGGCACCAATGATATT
GTTGTGAGCGACTGGAAAAAAGAGGATACCCTTTGGTATCTGAAA
AAGACCTGCCAGTACATCAAAAAAAAGAATGCAGCCAGCAAAATC
TACCTGCTGAGCGTTCCGCCTGTTTTTGGTCGTATTGATCGTGAT
AATCGTATCATCAATGACCTGAATAGCTATCTGCGTGAAAATGTG
GATTTCGCCAAATTTATCAGCCTGGATCATGTTCTGAAAGATTCC
TATGGCAACCTGAACAAAATGTATACCTATGATGGCCTGCACTTT
AACAGCAATGGTTATACCGTTCTGGAAAACGAAATTGCCGAGATC
GTTAAATGGAGCCATCCGCAGTTCGAAAAAGGTGGTGGTTCTGGT
GGTGGTTCTGGTGGTTCTGCGTGGAGCCATCCGCAGTTCGAAAAA
TAA
(SEQ ID NO: 15)
ATGCGCACCAAAATTATCGCAATTATTCCGGCACGTAGCGGTAGC
AAAGGTCTGCGTAACAAAAATGCACTGATGCTGATTGATAAACCG
CTGCTGGCATATACCATTGAAGCAGCACTGCAGAGCGAAATGTTT
GAAAAAGTTATTGTTACCACCGACAGCGAACAGTATGGTGCAATT
GCCGAAAGCTATGGTGCAGATTTTCTGCTGCGTCCGGAAGAACTG
GCAACCGATAAAGCAAGCAGCTTTGAATTTATCAAACATGCCCTG
AGCATCTATACCGATTATGAAAATTTTGCACTGCTGCAGCCGACC
AGTCCGTTTCGTGATAGCACCCATATTATTGAAGCCGTGAAACTG
TATCAGACCCTGGAAAAGTATCAGTGTGTTGTTAGCGTTACCCGT
AGCAATAAACCGAGCCAGATTATTCGTCCGCTGGATGATTATAGC
ACCCTGAGCTTTTTTGATCTGGACTACAGCAAATATAACCGCAAC
AGCATTGTTGAATATCATCCGAATGGTGCCATTTTTATCGCCAAC
AAACAGCATTATCTGCATACCAAACATTTTTTCGGTCGTTATAGC
CTGGCCTACATCATGGATAAAGAAAGCTCACTGGATATCGATGAT
CGCATGGATTTTGAACTGGCCATTACCATTCAGCAGAAGAAAAAT
CGTCAGAAGATTCTGTACCAGAACATCCATAATCGCATCAACGAA
AAACGCAACGAATTTGATAGCGTGAGCGATATTACCCTGATTGGT
CATAGCCTGTTTGATTATTGGGACGTGAAAAAGATCAACGACATC
GAAGTTAATAACCTGGGTATTGCCGGTATCAACAGCAAAGAATAT
TACGAGTACATCATCGAGAAAGAGCGCATTGTTAATTTTGGCGAA
TTCGTGTTTATCTTTTTCGGCACCAATGATATTGTTGTGAGCGAC
TGGAAAAAAGAGGATACCCTTTGGTATCTGAAAAAGACCTGCCAG
TACATCAAAAAAAAGAATGCAGCCAGCAAAATCTACCTGCTGAGC
GTTCCGCCTGTTTTTGGTCGTATTGATCGTGATAATCGTATCATC
AATGACCTGAATAGCTATCTGCGTGAAAATGTGGATTTCGCCAAA
TTTATCAGCCTGGATCATGTTCTGAAAGATTCCTATGGCAACCTG
AACAAAATGTATACCTATGATGGCCTGCACTTTAACAGCAATGGT
TATACCGTTCTGGAAAACGAAATTGCCGAGATCGTTAAATGGAGC
CATCCGCAGTTCGAAAAAGGTGGTGGTTCTGGTGGTGGTTCTGGT
GGTTCTGCGTGGAGCCATCCGCAGTTCGAAAAATAA
(SEQ ID NO: 16)

Plasmid DNA used in CFPS reactions was purified from glycerol stocks provided by Twist Biosciences using the ZymoPURE Midi Kit (Zymo Research D4200). Plasmids have been made available on Addgene (Table 3).

TABLE 3
Addgene ID's for plasmid accession. All plasmids used
in this study have been made accessible on Addgene. Addgene
ID and plasmid name are listed here for easy retrieval.

| Plasmid Name | Addgene ID |
|---|---|
| ApNGT.pJL1 | 199111 |
| LgtB.pJL1 | 199112 |
| CSTI.pJL1 | 199113 |
| PdST6.pJL1 | 199114 |
| NeuA.pJL1 | 199115 |
| Derp2.pJL1 | 199116 |

Synthesis of C5- and C9-Azido-Sialic Acids. To make C5-azido-sialic acid, N-Azidoacetylmannosamine (317 mg, 1.21 mmol) was dissolved in H₂O (12.1 mL), followed by addition of sodium pyruvate (665 mg, 5 equiv). Neu5Ac aldolase (8 U mg-1) was added, and the mixture was heated to 37° C. and stirred overnight. The solvents were evaporated, and the crude product was purified by column chromatography (0-20% 0.04 M HCl in MeCN, v/v), yielding the product (344.6 mg, 81.4%) as a white solid. 1H NMR (500 MHz, D2O): δ 4.16-4.06 (m, 4H), 4.01 (t, J=10.3 Hz, 1H), 3.84 (dd, J=11.8, 2.7 Hz, 1H), 3.75 (ddd, J=9.1, 6.3, 2.7 Hz, 1H), 3.62 (dd, J=11.9, 6.3 Hz, 1H), 3.56 (dd, J=9.3, 1.2 Hz, 1H), 2.33 (dd, J=13.1, 5.0 Hz, 1H), 1.89 (dd, J=13.1, 11.5 Hz, 1H). 13C NMR (126 MHz, D2O): δ 173.5, 171.0, 95.4, 70.2, 70.1, 68.2, 66.5, 63.1, 52.1, 51.9, 38.9 (FIG. 7).

C9-azido-sialic acid was synthesized as described previously. (64)

Harvest and Processing of E. coli Lysate for CFPS. E. coli lysate was prepared using previously published methods. (30) A highly productive engineered strain of MG1655-derived E. coli strain C321.ΔA.75956 was grown in a Sartorius Biostat C+ 10 L fermentor in 10 liters of 2×YTPG media (yeast extract 10 g/L, tryptone 16 g/L, NaCl 5 g/L, K2HPO4 7 g/L, KH2PO4 3 g/L, and glucose 18 g/L, pH=7.2) at 34° C. with rotation at 250 rpm. T7 RNA polymerase expression was induced with 1 mM isopropyl β-D-1-thiogalactopyranoside (Sigma-Aldrich) at OD=0.6. At OD=3.0, cells were put on ice and centrifuged at 8000 g for 5 min at 4° C. in a chilled JLA 8.1 rotor in a Beckman Avanti J-25I refrigerated centrifuge. The supernatant was discarded, and the cell pellets were washed three times with S30 buffer (10 mM trisacetate pH 8.2, 14 mM magnesium acetate, 60 mM potassium acetate) and pelleted by centrifugation at 10,000 g for 2 min at 4° C. in a Thermo Heraeus Multifuge X3R centrifuge. Cell pellets were flash frozen and stored at −80° C. After thawing, cells were resuspended in 0.8 mL of S30 buffer per gram of wet cell mass, distributed in 1.4 mL aliquots, and sonicated (Qsonica) at 50% amplitude for a cycle of 45 s on, 59 s off until ~950 Joules was reached. Lysate was kept on ice and spun at 12,000 g for 10 min at 4° C. The supernatant (820 μL) was collected in fresh tubes and incubated at 37° with shaking at 250 rpm for 1 h. Tubes were then spun at 12,000 g for 10 min at 4° C., and the supernatant collected from this final spin (500 μL) comprises the cell-free extract used to assemble CFPS reactions. The extract was flash frozen and stored at −80° C. until ready for use.

Assembly of CFPS Reactions. CFPS reactions were assembled based on previously published methods with the following building blocks and reagents. (30) 6 mM magnesium glutamate; 10 mM ammonium glutamate; 130 mM potassium glutamate; 2.646 mM ATP; 1.874 mM each of GTP, UTP, and CTP; 0.075 mg/mL folinic acid, 0.376 mg/mL E. coli tRNA mixture from strain MRE600 (Roche Applied Science); 0.33 mM nicotinamide adenine dinucleotide; 0.27 mM coenzyme-A (CoA); 4 mM oxalic acid; 1 mM putrescine; 1.5 mM spermidine; 57 mM HEPES at pH=7.2; 2 mM of each of the 20 standard amino acids; 30 mM phosphoenolpyruvate (Roche Applied Science); 13.3 μg/mL designed gene construct in the pJL1 vector; and 27% v/v of E. coli crude lysate (prepared above). Reagents were purchased from Sigma Aldrich unless otherwise stated. CSTI was expressed under oxidizing conditions, which was assembled as described above with an additional 30 min preincubation of the extract in 14.3 M iodoacetamide before supplementation into the CFPS reaction and with the additional following reagents: 4 mM oxidized L-glutathione GSSG, 1 mM reduced L-glutathione, and 3 μM purified DsbC from E. coli. CFPS reactions were incubated for 20 h at room temperature, with the exception of LgtB, which was flash frozen after 6 h to prevent protease degradation.

Radioactive Quantification of Proteins Expressed by CFPS. Radioactive quantification was performed based on previously published methods. (30, 65) In brief, CFPS reactions were assembled as described above with the addition of 10.67 μM radioactive amino acid, 14C-leucine. A negative control CFPS reaction where no DNA was added was included as a reference control. After 20 h of incubation, the samples were mixed with 0.5 N KOH—to precipitate the protein—in a 1:1 volume ratio (i.e., 5 μL of 0.5 N KOH and 5 μL of CFPS reaction) to quantify the total protein expression. Samples were then spun at 12,000 g for 10 min, and the supernatant was mixed with 0.5 N KOH in a 1:1 volume ratio to quantify the soluble protein expression. All CFPS+KOH samples were incubated for 20 min at 37° C. From each of these mixtures, 4 μL was dispensed onto a filtermat (PerkinElmer, 1450-421) and duplicated on a second filtermat and allowed to dry for 20 min. One filtermat for each set of samples was washed 3× in 5% trichloroacetic acid for 15 min at 4° C., while the other filtermat was left alone. A sheet of scintillation wax was melted on a hot plate over the filtermat, and the radioactive signal was read from each sample by the Microbeta2 scintillation counter.

Protein yields (μg/mL) are calculated by the following formula:

((Signal of sample on washed filtermat signal of negative control sample)/signal of sample on unwashed filtermat)*[14C-leucine(μM)]*molecular weight of protein(g/mol)/(#leucine residues×1000)

Protein yields (μg/mL) can be converted to (μM) by the following formula:

(1000*protein yield μg/mL)/molecular weight(g/mol)

Assembly of IVA Reactions. CFPS reactions were centrifuged at 12,000 g for 10 min, and the supernatant was transferred to the final IVA reaction. Lysates were mixed in optimized proportions, where the target glycoprotein com-prised 45% of the final reaction volume, ApNGT comprised 2%, LgtB 7.4%, CSTI/PdST6 13%, and NeuA (when included) 4.5%. Activated sugar substrates (UDP-glucose, UDP-galactose, and CMP-sialic acid) were supplemented at a concentration of 2.5 mM. Nonactivated sialic acid and CTP were supplemented at 7.5 mM and 45 μM, respectively. For incorporation of azido-sialic acids, C9-azido-sialic acid should be supplemented at 7.5 mM with CTP at 45 μM and C5-azido-sialic acid should be supplemented at 125 mM with CTP at 750 μM. IVA assemblies were incubated at 30° C. for 24 h, at which time sugar substrates were resupplemented at their optimized concentration and the reactions were incubated for another 24 h at 30° C.

Glycoprotein Purification. IVA reactions were diluted in 50 mM NaH2PO4, 300 mM NaCl, pH=8.0 (Buffer 1) and centrifuged at 12,000 g for 10 min. His-tagged, glycosylated Der p 2 allergen was isolated by affinity-tag purification with Ni-NTA magnetic beads (Invitrogen Dynabeads His-tag Isolation and Pulldown, 10104D). Beads were equilibrated with Buffer 1, then resuspended in the IVA reaction supernatant, and allowed to bind for >10 min at 4° C. with end-over-end rotation. Samples were then washed twice with Buffer 1 and eluted with 500 mM imidazole in Buffer 1 with 10 min of end-over-end mixing at room temperature.

The elution was desalted using Zeba spin columns (Thermo Scientific 89883) according to manufacturer's instructions and collected in nuclease free water (Ambion, AM9937).

LC-MS Analysis of Glycoprotein. After the samples were trypsinized with 0.0044 μg/μL MS grade trypsin (Thermo Fisher Scientific, PI90057) with an incubation of at least 4 h at 37° C., the samples were run on the Bruker Impact II ESI QTOF. Samples were first passed through an ACUITY UPLC Peptide BEH C18 column (300 Å, 1.7 μM, 2.1 mm×100 mM, 186003686 Waters Corp.) equipped with a 10 mm guard column of identical packing (186004629 Waters Corp.). Then, the samples were eluted at a flow rate of 0.5 mL/min and a 40° C. column temperature starting at a 1 min hold of 95% solvent A (100% water and 0.1% formic acid) and 5% solvent B (100% acetonitrile and 0.1% formic acid) with a linear gradient up to 0% solvent A and 100% solvent B for a total run time of 9 min.

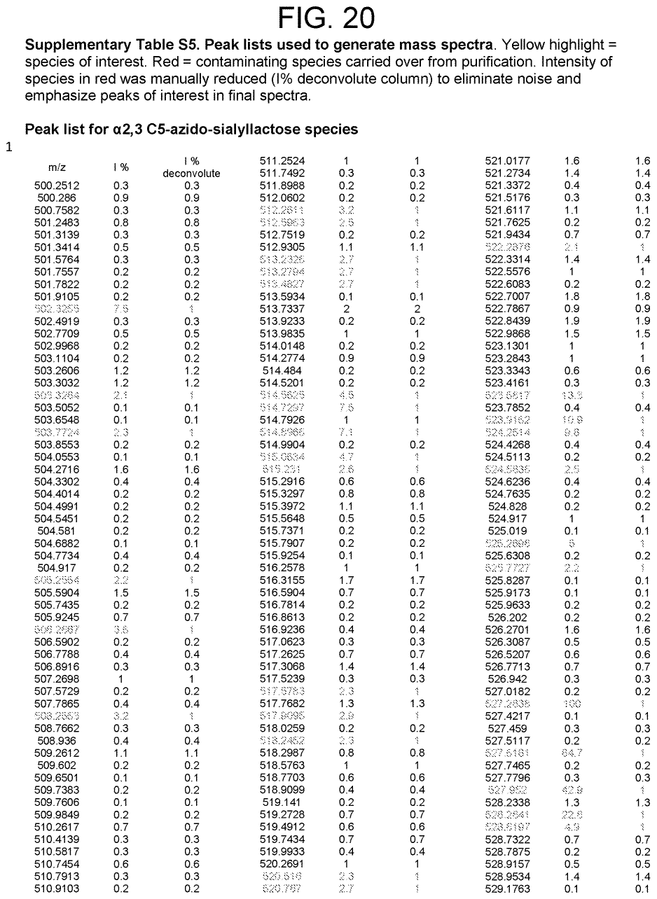

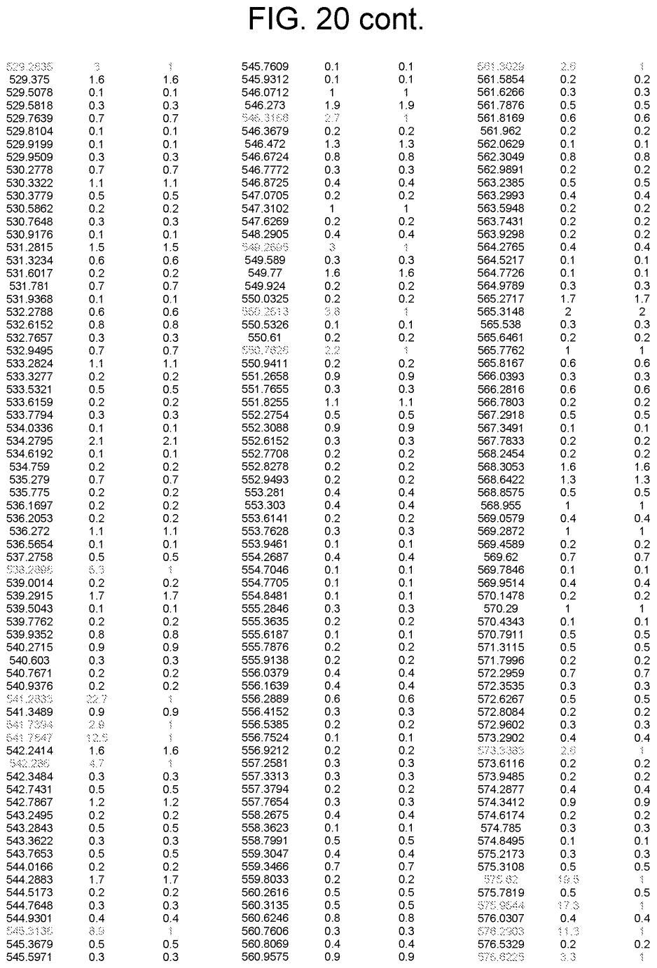

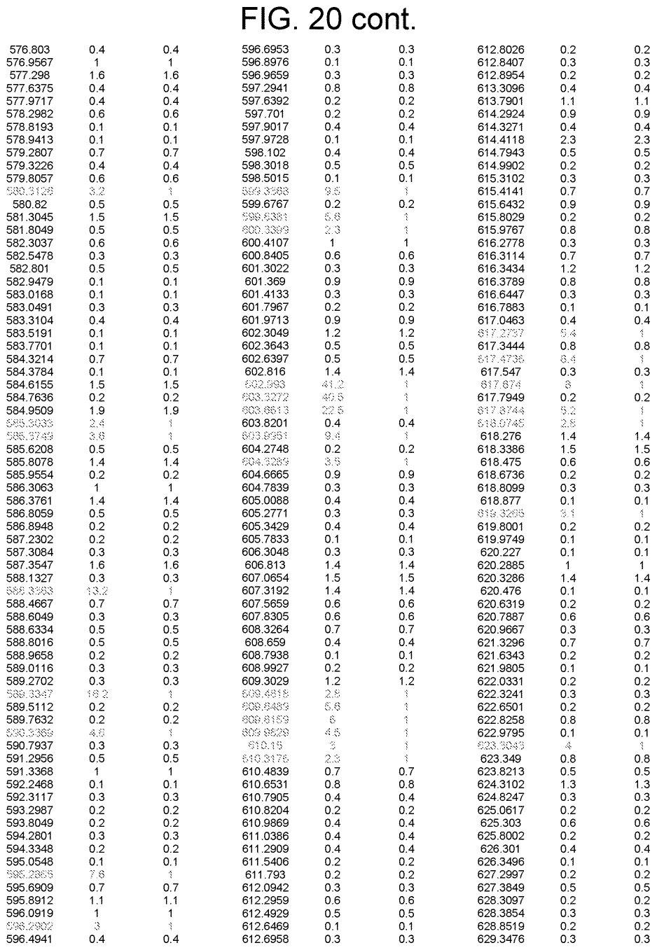

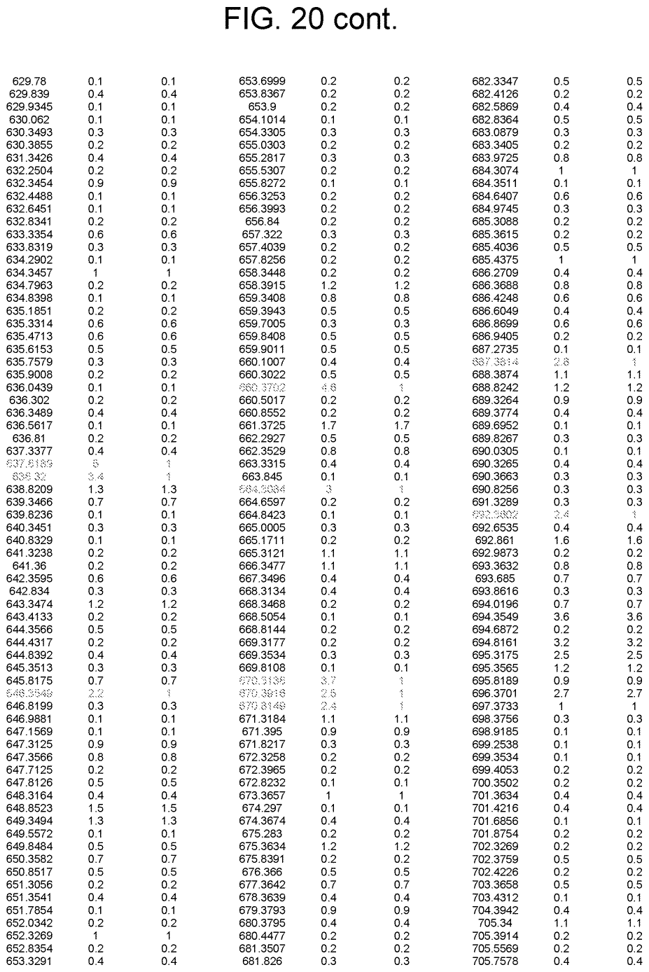

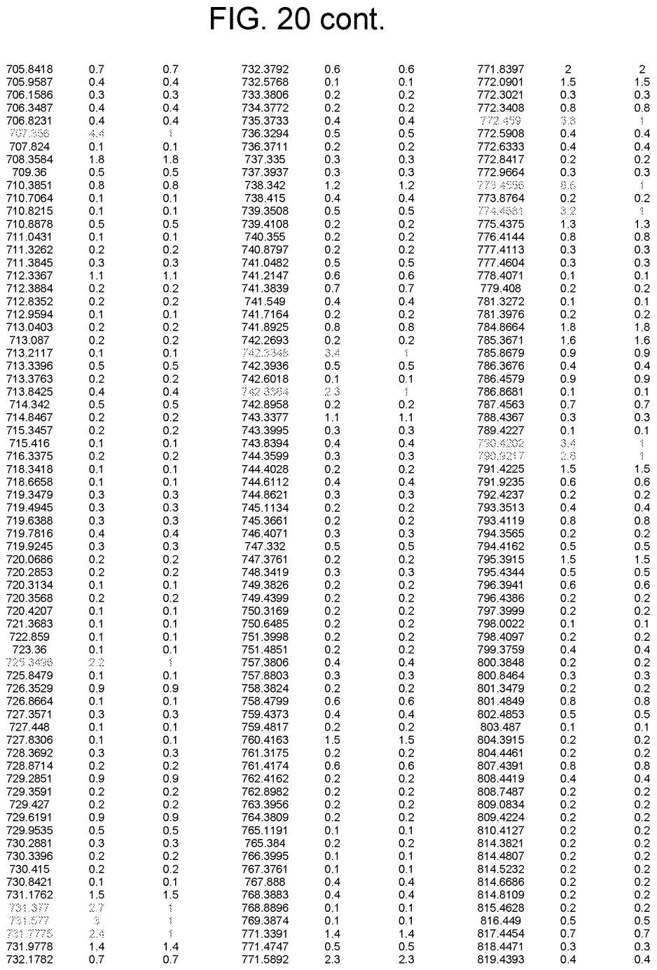

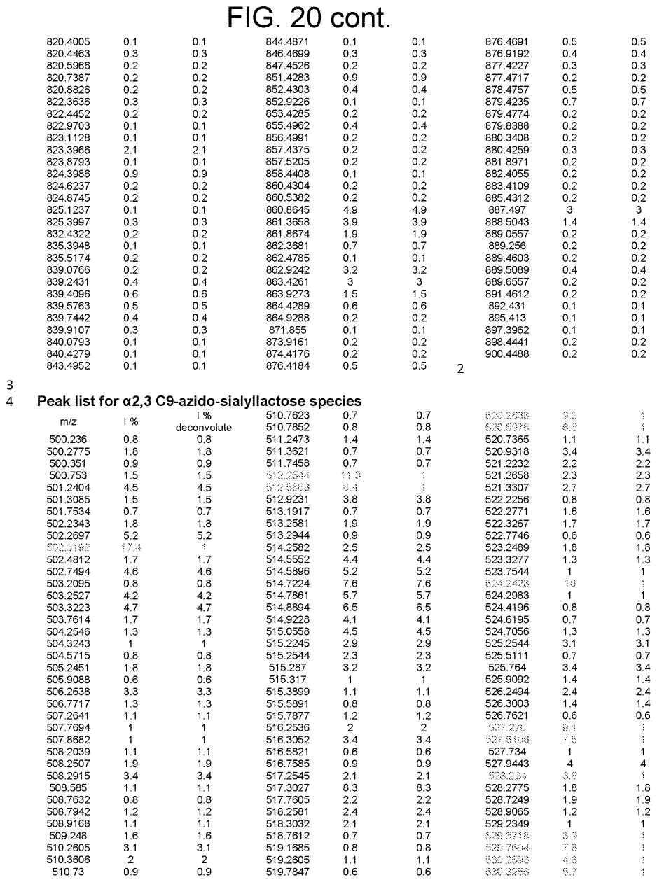

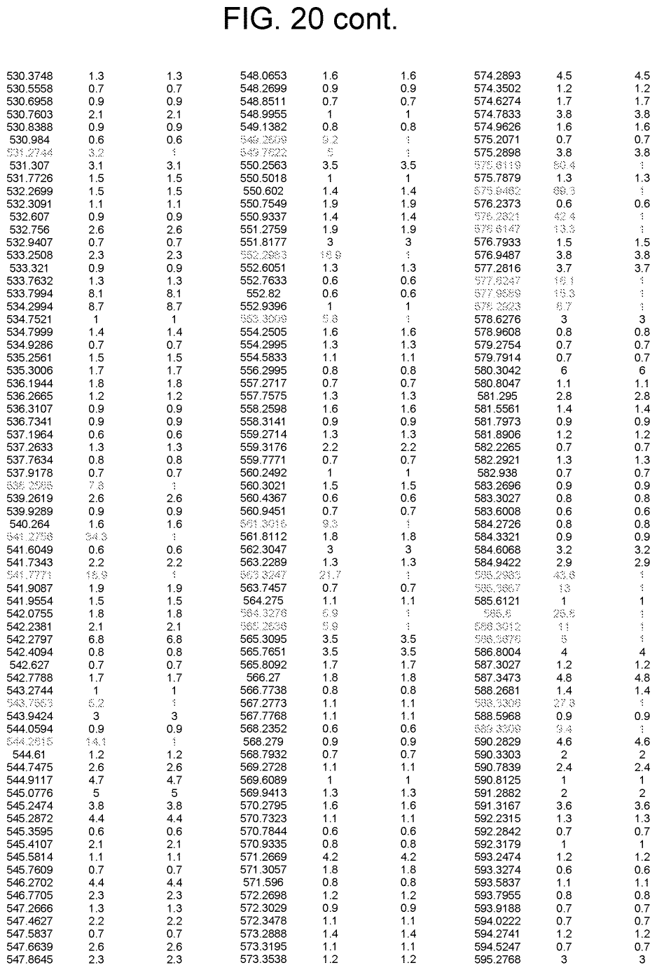

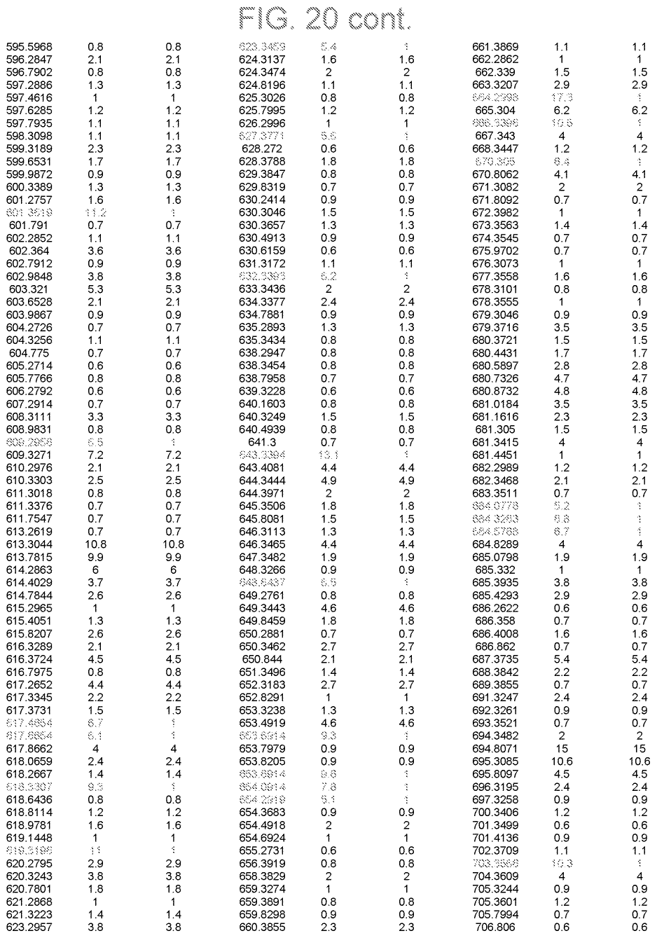

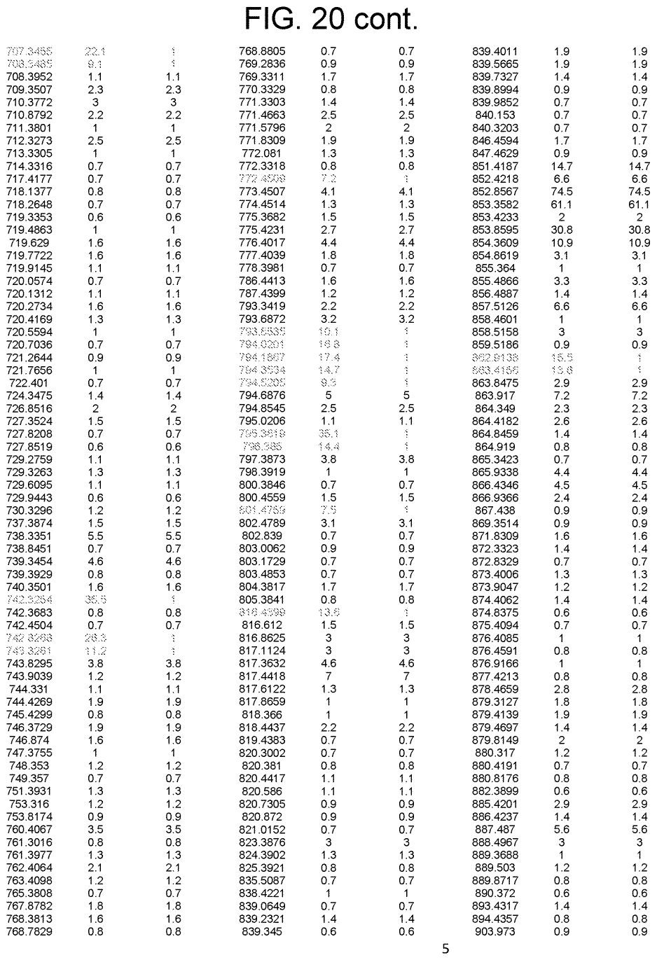

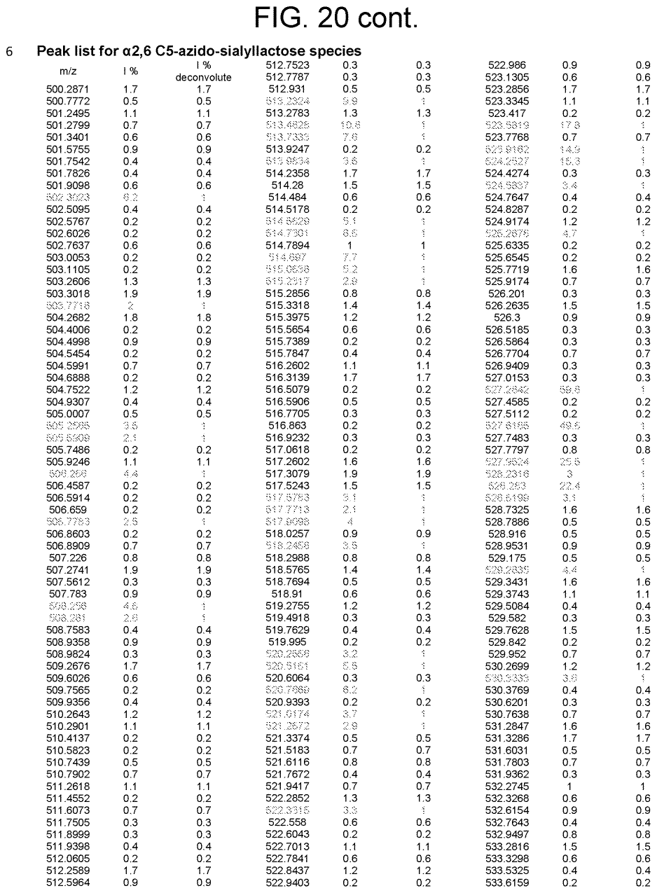

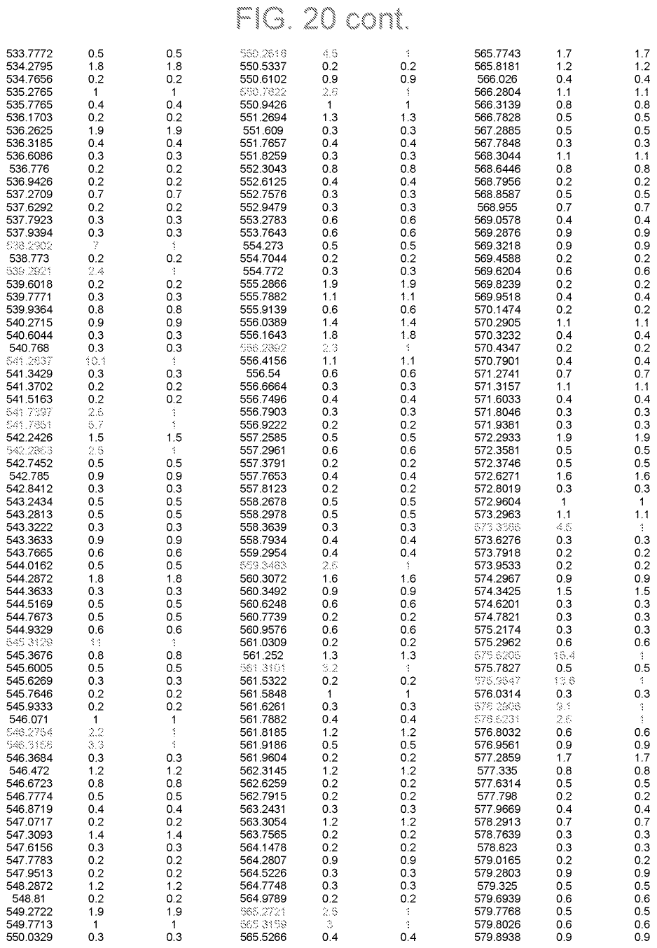

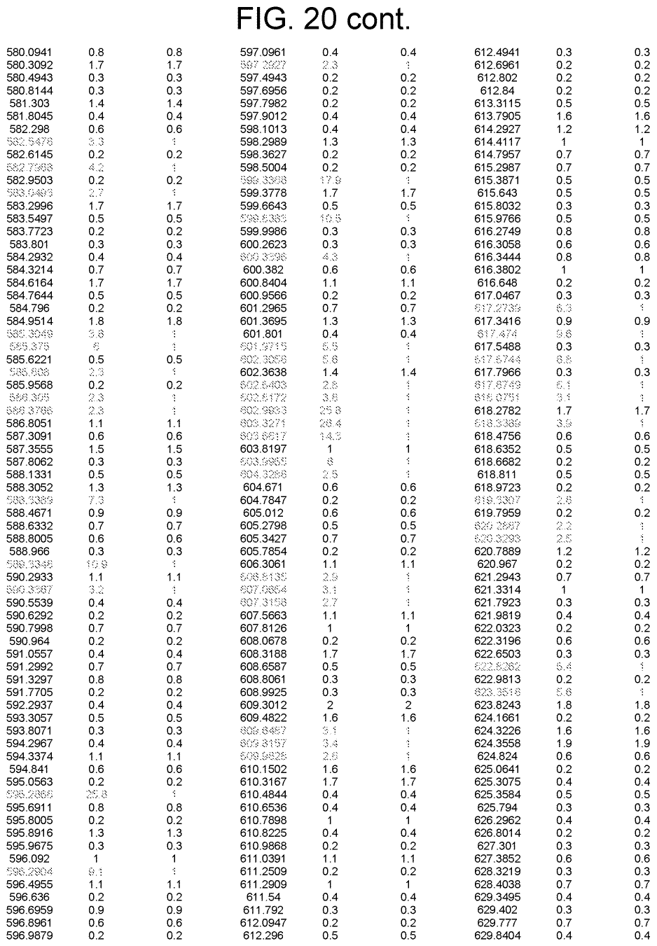

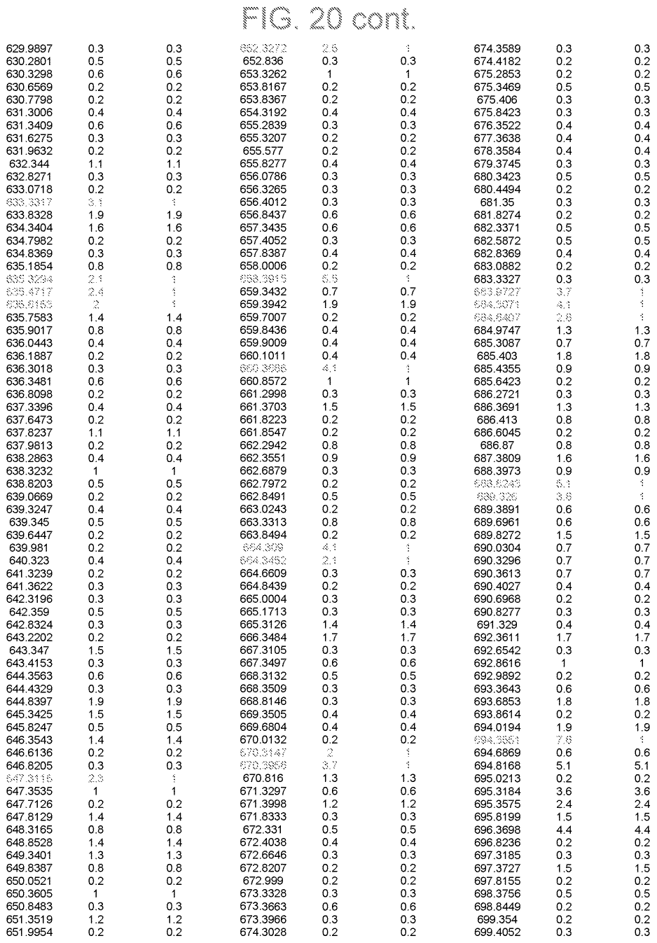

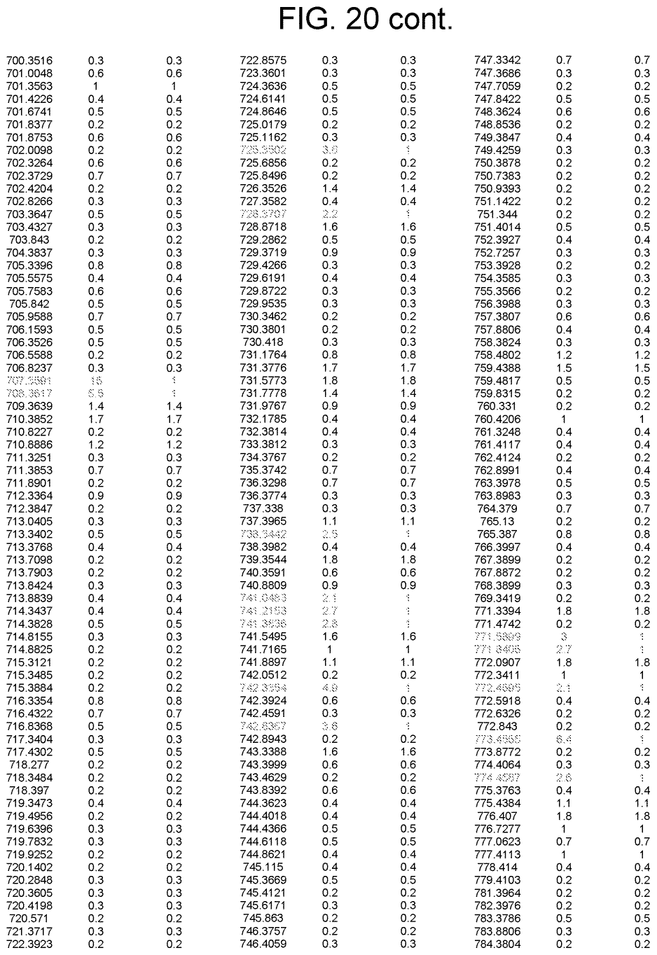

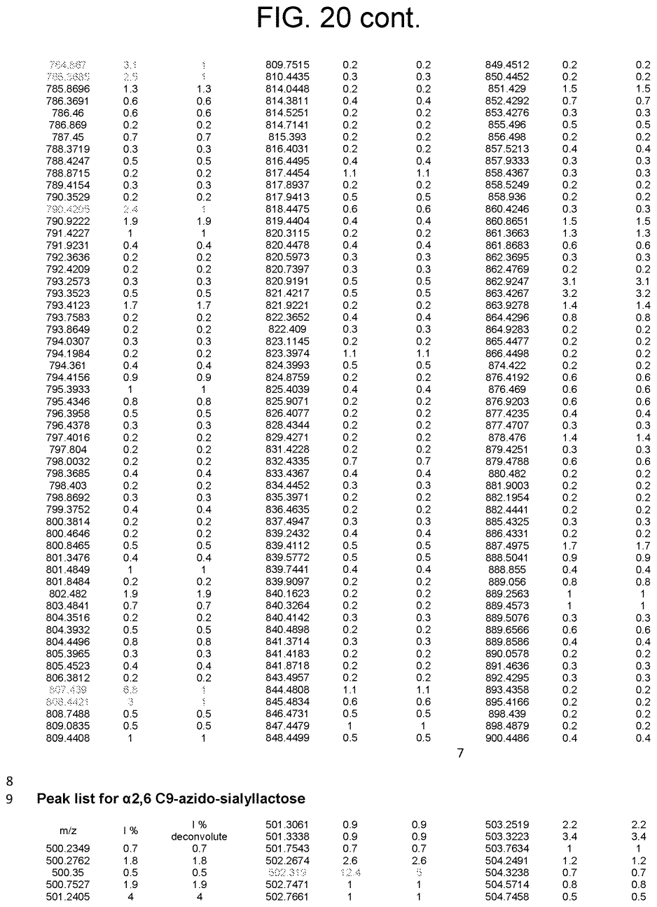

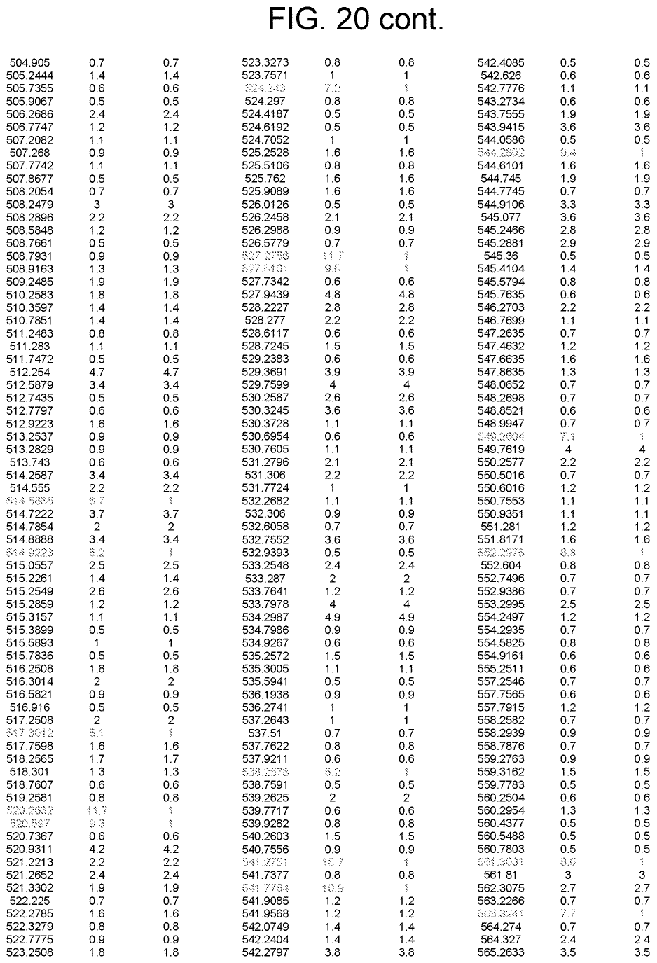

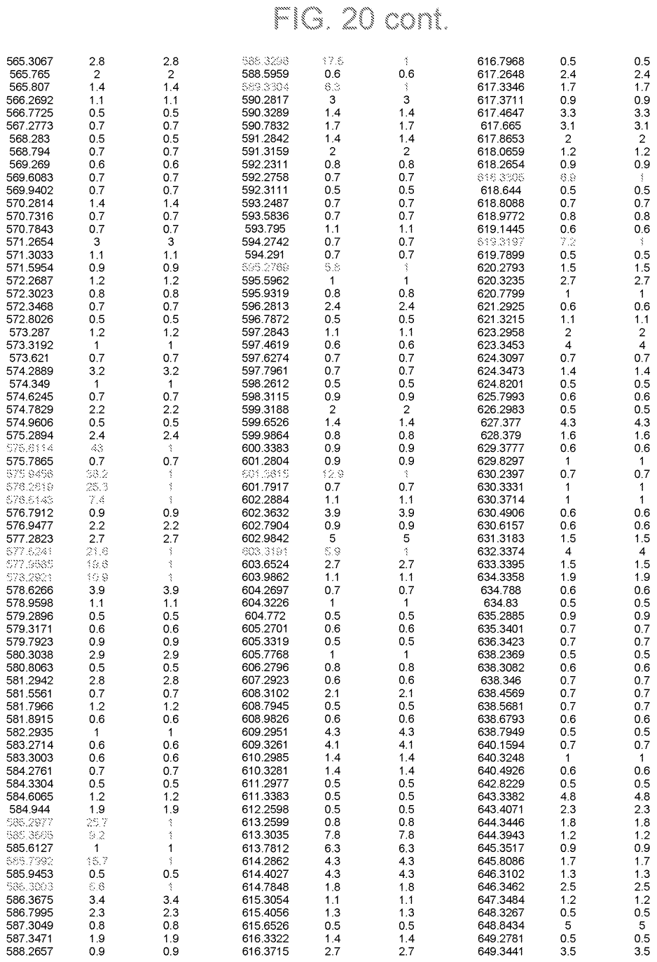

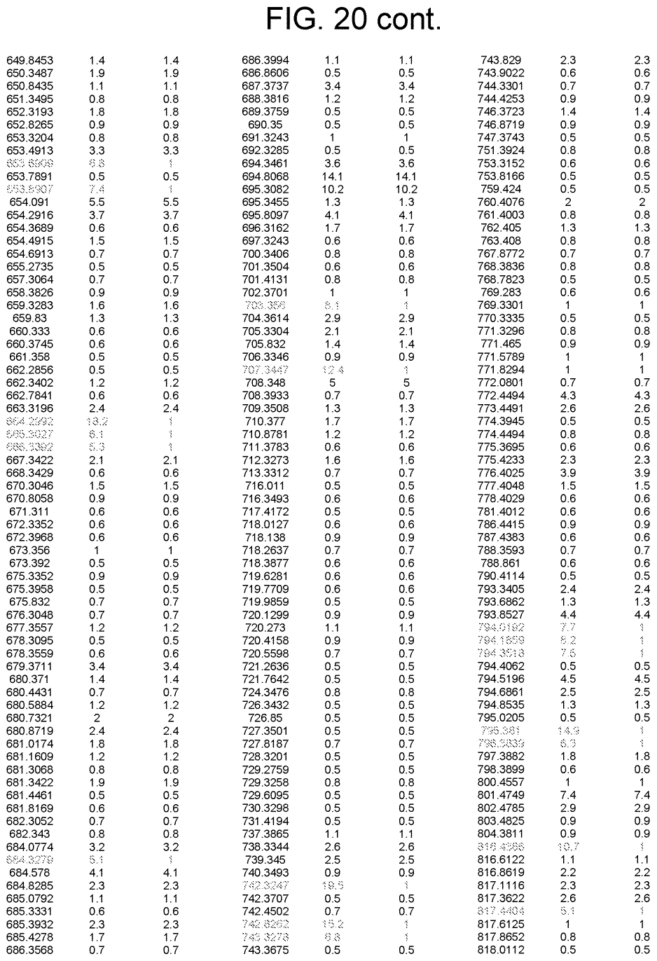

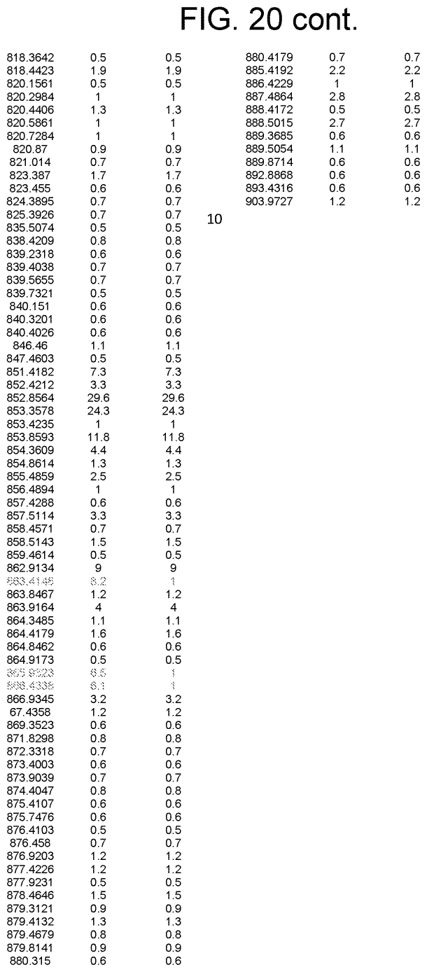

MS Data Analysis. MS data were analyzed using the Bruker software. Theoretical masses in the +2 charge state (Table 4), which was determined to be the predominant form, were used to make the extracted ion chromatogram (EIC) for each pathway intermediate and final product. Glycosylation efficiency was determined by taking the area under the curve (AUC) of each intermediate's EIC and calculating the relative percentage represented by the AUC of a given intermediate relative to summed AUCs of all possible intermediates. Spectra were plotted using m/z values from 500 to 900 at the elution times encompassing EICs and deconvoluted to emphasize species of interest (FIG. 20).

TABLE 4
Theoretical m/z values. Calculated m/z values were used to make
extracted ion chromatograms for each species to calculate area
under the curve as an estimation of glycosylation efficiency.
Sugars were installed onto the asparagine within the peptide
sequence ATTGGNWTTR (SEQ ID NO: 19). Glycoprotein samples were
trypsinized to yield this remaining glycopeptide sequence.

| | Theoretical m/z in +2 |
|---|---|
| Species | charge state |
| Aglycosylated peptide | 532.7596 |
| Glucosylated peptide | 613.78665 |
| Lactosylated peptide | 694.81165 |
| Sialyllactosylated peptide | 840.35935 |
| C5-azido-sialyllactosylated peptide | 860.94601 |
| C9-azido-sialyllactosylated peptide | 852.94651 |

GlycoCAP at Preparative Scale. CFPS reactions were assembled using conditions described above at sufficient volumes for addition into IVA reactions. IVA reactions were assembled at a total volume of 1 mL and were comprised of CF-expressed components in optimized relative proportions and the necessary substrates and cofactors at optimum molar concentration as described above (FIG. 2). After incubation and supplementation as described for IVA assembly, His-tagged glycoprotein was purified with Ni-NTA magnetic beads (Invitrogen Dynabeads His-tag Isolation and Pulldown, 10104D). Purified samples were desalted as described above and analyzed by MS for glycosylation efficiency. Protein yield was determined by UV absorbance at 280 nM using a Thermo Nanodrop 2000 spectrophotometer (extinction coefficient: 15470, molecular weight: 17376 Da).

Click Chemistry Conjugation. Click chemistry reactions were assembled using purified glycoprotein at a total volume of 100 μL. For strain-promoted click chemistry, a stock of 3.4 mM DBCO-PEG4-Fluor 545 (Sigma-Aldrich, 760773) sus-pended in dimethyl sulfoxide was added to the purified and desalted glycoprotein in water (~250 μg/mL) at a concentration of 400 μM and allowed to incubate at 30° C. for 4 h.

For copper-catalyzed click chemistry, ~10 μg of purified, desalted glycoprotein product suspended in 100 μL of 1% RapiGest (Waters, 186008090) was combined with 300 μM CuSO4, 600 μM BTTP (Click Chemistry Tools, 1414-100), and 200 μM Alexa Fluor 546-Alkyne (Jena Bioscience, CLK-1285-1) and allowed to incubate for at least 1 h at room temperature to allow the ligand complex to form. Sodium ascorbate (2.5 mM) was then added the reaction, and the reaction underwent an incubation period of 3.5 h at room temperature.

Fluorescent Gel. Licor protein sample loading buffer (LI-COR Biosciences 928-40004) and 4 mM DTT were added directly to click chemistry conjugation reactions containing the click-modified glycoprotein. Samples were heat denatured at 70° C. for 3 min and then run on a NuPAGE 4-12% Bis-Tris gel (Invitrogen, NP0321BOX) at 150 V with MOPS buffer (Thermo Fisher Scientific, NP0001). Gel was imaged on a LI-COR-Odyssey Fc imaging system on the 600 and 800 channels.

### Example 2. Additional Data on a Cell-Free Glycosylation Platform for Clickable Azido-Sialoglycanproduction

The platform in this Example is a bacterial, cell free glycosylation platform that allows for the production of clickable azido-sialoglycan structures, among other non-canonical glycan structures, similar to that described above with reference to Example 1. E. coli have no endogenous glycosylation machinery, so they provide a blank slate for glycoengineering. This allows for site specific control without competition from other glycosylation enzymes and yields highly homogeneous glycan populations. The cell free system provides an open reaction environment, which offers the ability to tune reaction conditions to optimize expression and activity of each component. Importantly, it also allows supplementation of non-canonical sugars, including azido-sialic acid, without being constrained by possible toxicity to living cells. The platform utilizes lysate harvested from E. coli to express each component of the glycosylation pathway using Cell Free Protein Synthesis (CFPS). After expression, each component is mixed together in optimized proportions in an In Vitro Glycosylation Assembly (IVA) reaction, where sugar substrates and appropriate cofactors are supplemented. After incubation, a glycosylated protein can be purified out and used directly from the IVA for analysis, treatment, and production. The main procedures for CFPS and IVA and described above with reference to Example 1.

In this Example, the components of the glycosylation pathway include a protein (e.g., an allergen, antibody, therapeutic, etc.) that is designed with an amino acid sequence recognized by ApNGT for glycosylation, otherwise known as a Glyctag (e.g., GGNWTT (SEQ ID NO: 18)). At that site, ApNGT (from Actinobacillus pleuropneumoniae) will install a glucose from UDP-glucose onto an asparagine. LgtB (from Neisseria gonorrhoeae) elaborates a galactose from UDP-galactose onto the glucose in a β1,4 linkage. CSTI (from Campylobacter jejuni) elaborates a sialic acid from CMP-sialic acid on to the galactose in an α2.3 linkage and PdST6 (from Photobacterium damselae) elaborates a sialic acid from CMP-sialic acid on to the galactose in an α2.6 linkage. NeuA catalyzes the formation of CMP-sialic acid from a sialic acid or non-canonical sialic acid (like azido-sialic acid) and CTP to form an appropriate substrate for the CSTI and PdST6 sialyltransferases.

The platform utilized in this Example will site-specifically install a sialyllactose structure onto any protein with an encoded Glyctag, where the sialic acid is in either an α2.3 linkage or an α2.6 linkage to galactose and where galactose is in a β1,4 linkage to the glucose. Glycans will be linked to the protein through a hydrolysis reaction where the 1^{st} carbon of the glucose will be bonded to the nitrogen of the asparagine R group. Incorporation of non-canonical sugars also allows the production of C5-azido sialyllactose where an azide group is on the 5th carbon of the sialic acid or a C9-azido sialyllactosewhere an azide group is on the 9th carbon of the sialic acid. The azide group provides a handle for click chemistry conjugation where any moiety (e.g., chemical group, protein, nucleic acid, carbohydrate, etc.) with a standard alkyne or strained alkyne can be conjugated to the protein with the formation of an intervening ring.

DNA design of the glycoprotein. In this Example, the design scheme uses the dust mite allergen Der p 2, which is designed with a C-terminal CAT linker to standardize ribosome binding and expression. An N-terminal glyctag is separated from the protein by a BamHI restriction site linker and an affinity tag (either superflag and Strep separated by Pas12 peptide linkers, or a His tag) follows the Glyctag. The sequence terminates in a stop (TAA) codon. This sequence was cloned into a PJL1 backbone. The format for this superflag and Strep tag version is: Cat linker-Protein-BamHI linker-Glyctag-Pas12 linker-superflag tag-Pas12 linker-Strep tag-Stop codon. The DNA sequence for this construct is provided in SEQ ID NO: 1. The format for the His tag version is: Cat linker-Protein-BamHI linker-Glyctag-His tag-Stop codon. The DNA sequence for this construct is provided in SEQ ID NO: 2. This scheme can be applied to other proteins, including other allergens, antibodies, carrier proteins, etc. As an example, this scheme was applied to clinically relevant allergens where the sequences of SEQ ID NOs: 3-14 can replace the text shown in black in the Der p 2 examples. SEQ ID NO: 3 is for the Ara h 2 allergen protein. SEQ ID NO: 4 is for the Der p 1 allergen protein. SEQ ID NO: 5 is for the Fel d 1 allergen protein. SEQ ID NO: 6 is for the Amb a 1 allergen protein. SEQ ID NO: 7 is for the Bos d 5 allergen protein. SEQ ID NO: 8 is for the Bos d 4 allergen protein. SEQ ID NO: 9 is for the Gal d 1 allergen protein. SEQ ID NO: 10 is for the Gal d 2 allergen protein. SEQ ID NO: 11 is for the Gal d 4 allergen protein. SEQ ID NO: 12 is for the Bla g 2 allergen protein. SEQ ID NO: 13 is for the Cry j 1 allergen protein. SEQ ID NO: 14 is for the Bet v 1 allergen protein.

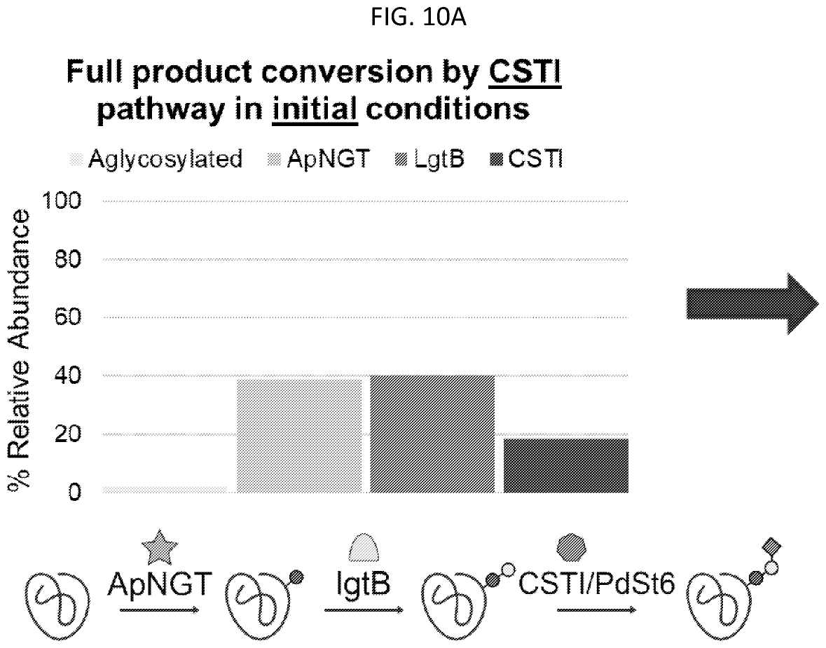

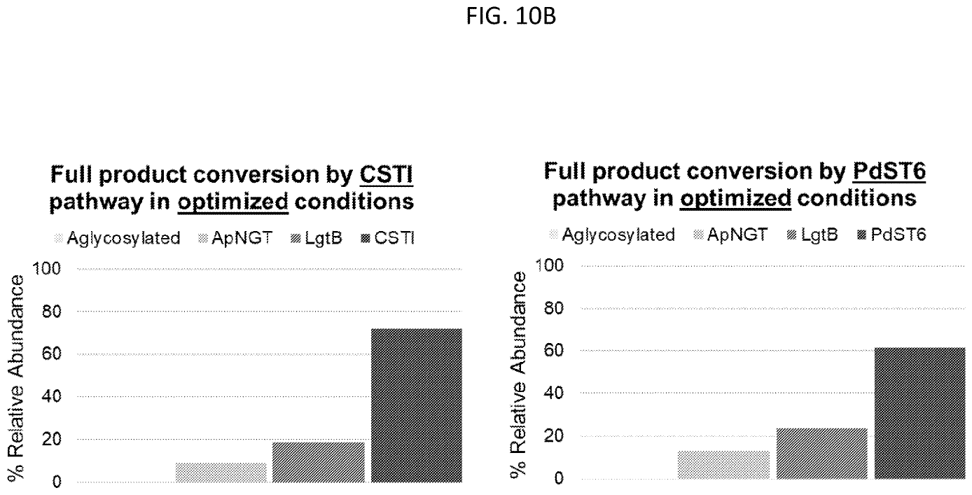

Optimization of the base pathway. An optimal set of conditions was used to establish a benchmark for yields based on “base conditions” without the added step of NeuA activation of sialic acid. These experiments were conducted using similar procedures to those provided above with reference to Example 1. With initial conditions, the sialylated final product represents ~20% of the total reaction products. The remaining 80% is comprised of the precursor reaction intermediates. (FIG. 10A). After a series of optimizations that includes (i) flash freezing LgtB after 6 hours of expression to prevent degradation products from protease activity (ii) adding flanking linker sequences to the Glyctag, and (iii) reducing precipitation induced by the HEPES buffer, the reaction yield a final product proportion of ~70% for the pathway with CSTI and ~60% for the pathway with PdST6. (FIG. 10B.)

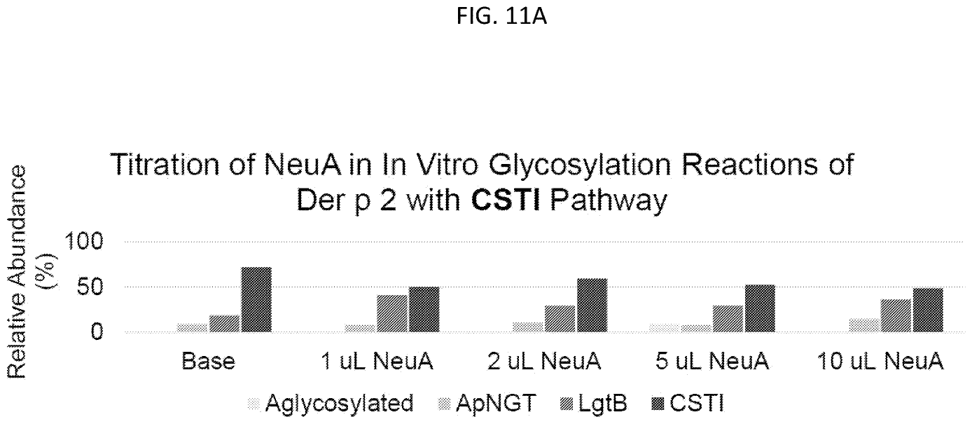

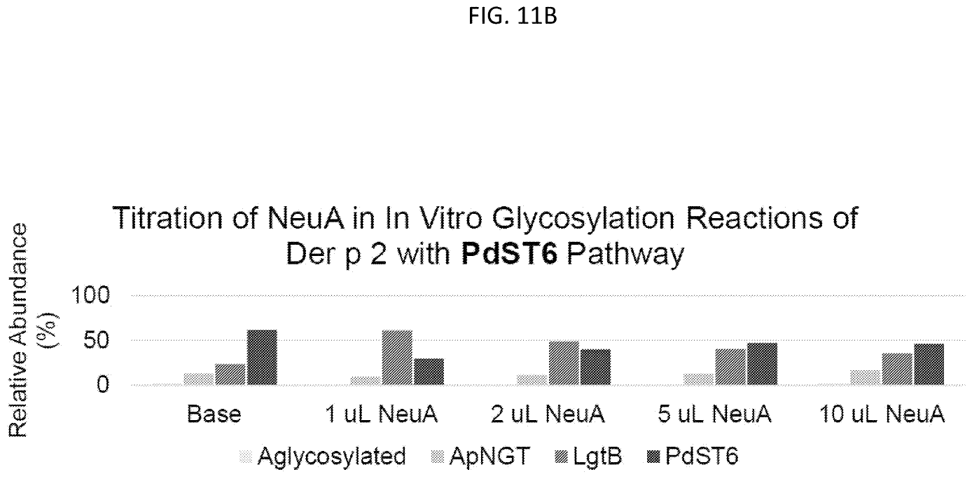

Optimization of NeuA. Addition to Base Pathway. A titration of CFPS lysate expressing NeuAwas performed to determine the amount that achieves highest yields of sialylated product. These experiments were conducted using similar procedures to those provided above with reference to Example 1. (FIG. 11A) When 2 μL of the NeuA CFPS reaction is added to the IVA containing CSTI pathway components, sialylated product makes up ~60% of the reaction product. (FIG. 11B) 5 μL of CFPS lysate expressing NeuA is required to achieve optimal yields in the IVA containing PdST6 pathway components (~40%). These yields are very high, but do not quite match the yields of base case conditions.

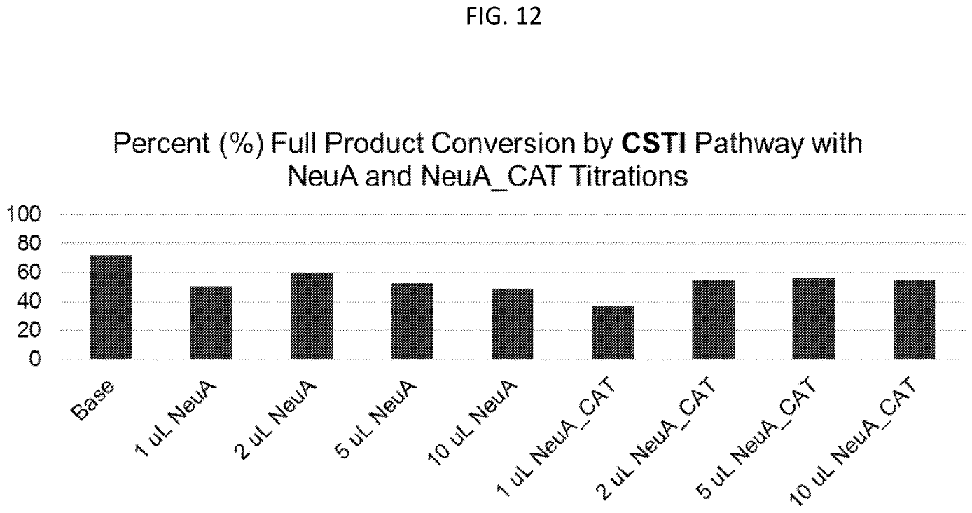

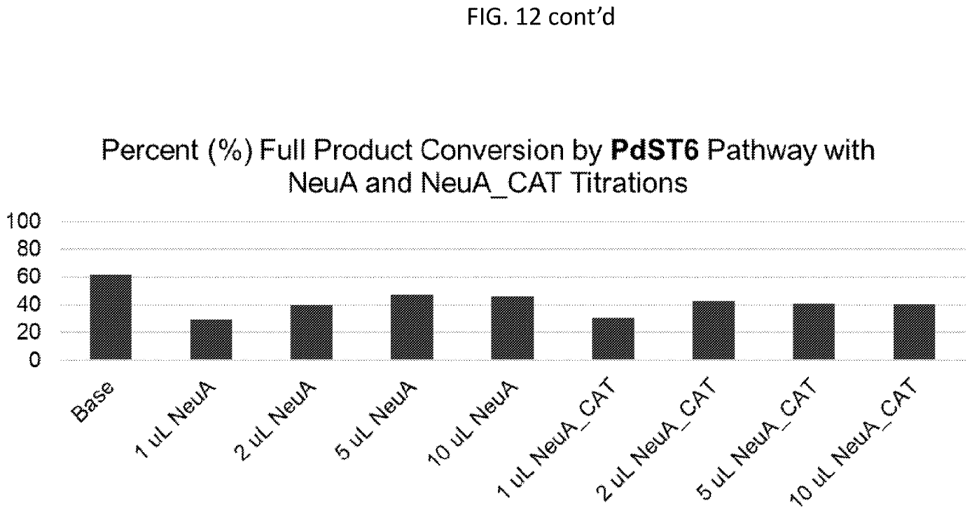

NeuA does not require a CAT linker for optimal activity. Two versions of NeuA sequences were produced in CFPS: one preceded by a CAT linker (orange) (top) (SEQ ID NO: 15) and one only with a start codon (bottom) (SEQ ID NO: 16), each cloned into a PJL1 backbone, found in the below Table 5. Each version was expressed by CFPS and titrated into the IVA. (FIG. 12) Yields of sialylated product do not vary significantly with equivalent volumes of CFPS lysate when NeuA is designed with a CAT linker (right) or without (left). This is true in both the CSTI (top) and PdST6 (bottom) pathways IVA's.

TABLE 5
NeuA sequences with and without CAT linker
NeuA Sequences (underlining is CAT linker)

| | |
|---|---|
| SEQ ID | ATGGAGAAAAAAATCCGCACCAAAATTATCGCAAT |
| NO: 15 | TATTCCGGCACGTAGCGGTAGCAAAGGTCTGCGTA |
| | ACAAAAATGCACTGATGCTGATTGATAAACCGCTG |
| | CTGGCATATACCATTGAAGCAGCACTGCAGAGCGA |
| | AATGTTTGAAAAAGTTATTGTTACCACCGACAGCG |
| | AACAGTATGGTGCAATTGCCGAAAGCTATGGTGCA |
| | GATTTTCTGCTGCGTCCGGAAGAACTGGCAACCGA |
| | TAAAGCAAGCAGCTTTGAATTTATCAAACATGCCC |
| | TGAGCATCTATACCGATTATGAAAATTTTGCACTG |
| | CTGCAGCCGACCAGTCCGTTTCGTGATAGCACCCA |
| | TATTATTGAAGCCGTGAAACTGTATCAGACCCTGG |
| | AAAAGTATCAGTGTGTTGTTAGCGTTACCCGTAGC |
| | AATAAACCGAGCCAGATTATTCGTCCGCTGGATGA |
| | TTATAGCACCCTGAGCTTTTTTGATCTGGACTACA |
| | GCAAATATAACCGCAACAGCATTGTTGAATATCAT |
| | CCGAATGGTGCCATTTTTATCGCCAACAAACAGCA |
| | TTATCTGCATACCAAACATTTTTTCGGTCGTTATA |
| | GCCTGGCCTACATCATGGATAAAGAAAGCTCACTG |
| | GATATCGATGATCGCATGGATTTTGAACTGGCCAT |
| | TACCATTCAGCAGAAGAAAAATCGTCAGAAGATTC |
| | TGTACCAGAACATCCATAATCGCATCAACGAAAAA |
| | CGCAACGAATTTGATAGCGTGAGCGATATTACCCT |
| | GATTGGTCATAGCCTGTTTGATTATTGGGACGTGA |
| | AAAAGATCAACGACATCGAAGTTAATAACCTGGGT |
| | ATTGCCGGTATCAACAGCAAAGAATATTACGAGTA |
| | CATCATCGAGAAAGAGCGCATTGTTAATTTTGGCG |
| | AATTCGTGTTTATCTTTTTCGGCACCAATGATATT |
| | GTTGTGAGCGACTGGAAAAAAGAGGATACCCTTTG |
| | GTATCTGAAAAAGACCTGCCAGTACATCAAAAAAA |
| | AGAATGCAGCCAGCAAAATCTACCTGCTGAGCGTT |
| | CCGCCTGTTTTTGGTCGTATTGATCGTGATAATCG |
| | TATCATCAATGACCTGAATAGCTATCTGCGTGAAA |
| | ATGTGGATTTCGCCAAATTTATCAGCCTGGATCAT |
| | GTTCTGAAAGATTCCTATGGCAACCTGAACAAAAT |
| | GTATACCTATGATGGCCTGCACTTTAACAGCAATG |
| | GTTATACCGTTCTGGAAAACGAAATTGCCGAGATC |
| | GTTAAATGGAGCCATCCGCAGTTCGAAAAAGGTGG |
| | TGGTTCTGGTGGTGGTTCTGGTGGTTCTGCGTGGA |
| | GCCATCCGCAGTTCGAAAAATAA |
| SEQ ID | ATGCGCACCAAAATTATCGCAATTATTCCGGCACG |
| NO: 16 | TAGCGGTAGCAAAGGTCTGCGTAACAAAAATGCAC |
| | TGATGCTGATTGATAAACCGCTGCTGGCATATACC |
| | ATTGAAGCAGCACTGCAGAGCGAAATGTTTGAAAA |
| | AGTTATTGTTACCACCGACAGCGAACAGTATGGTG |
| | CAATTGCCGAAAGCTATGGTGCAGATTTTCTGCTG |
| | CGTCCGGAAGAACTGGCAACCGATAAAGCAAGCAG |
| | CTTTGAATTTATCAAACATGCCCTGAGCATCTATA |
| | CCGATTATGAAAATTTTGCACTGCTGCAGCCGACC |
| | AGTCCGTTTCGTGATAGCACCCATATTATTGAAGC |
| | CGTGAAACTGTATCAGACCCTGGAAAAGTATCAGT |
| | GTGTTGTTAGCGTTACCCGTAGCAATAAACCGAGC |
| | CAGATTATTCGTCCGCTGGATGATTATAGCACCCT |
| | GAGCTTTTTTGATCTGGACTACAGCAAATATAACC |
| | GCAACAGCATTGTTGAATATCATCCGAATGGTGCC |
| | ATTTTTATCGCCAACAAACAGCATTATCTGCATAC |
| | CAAACATTTTTTCGGTCGTTATAGCCTGGCCTACA |
| | TCATGGATAAAGAAAGCTCACTGGATATCGATGAT |
| | CGCATGGATTTTGAACTGGCCATTACCATTCAGCA |
| | GAAGAAAAATCGTCAGAAGATTCTGTACCAGAACA |
| | TCCATAATCGCATCAACGAAAAACGCAACGAATTT |
| | GATAGCGTGAGCGATATTACCCTGATTGGTCATAG |
| | CCTGTTTGATTATTGGGACGTGAAAAAGATCAACG |
| | ACATCGAAGTTAATAACCTGGGTATTGCCGGTATC |
| | AACAGCAAAGAATATTACGAGTACATCATCGAGAA |
| | AGAGCGCATTGTTAATTTTGGCGAATTCGTGTTTA |
| | TCTTTTTCGGCACCAATGATATTGTTGTGAGCGAC |
| | TGGAAAAAAGAGGATACCCTTTGGTATCTGAAAAA |
| | GACCTGCCAGTACATCAAAAAAAAGAATGCAGCCA |
| | GCAAAATCTACCTGCTGAGCGTTCCGCCTGTTTTT |
| | GGTCGTATTGATCGTGATAATCGTATCATCAATGA |
| | CCTGAATAGCTATCTGCGTGAAAATGTGGATTTCG |
| | CCAAATTTATCAGCCTGGATCATGTTCTGAAAGAT |
| | TCCTATGGCAACCTGAACAAAATGTATACCTATGA |
| | TGGCCTGCACTTTAACAGCAATGGTTATACCGTTC |
| | TGGAAAACGAAATTGCCGAGATCGTTAAATGGAGC |
| | CATCCGCAGTTCGAAAAAGGTGGTGGTTCTGGTGG |
| | TGGTTCTGGTGGTTCTGCGTGGAGCCATCCGCAGT |
| | TCGAAAAATAA |

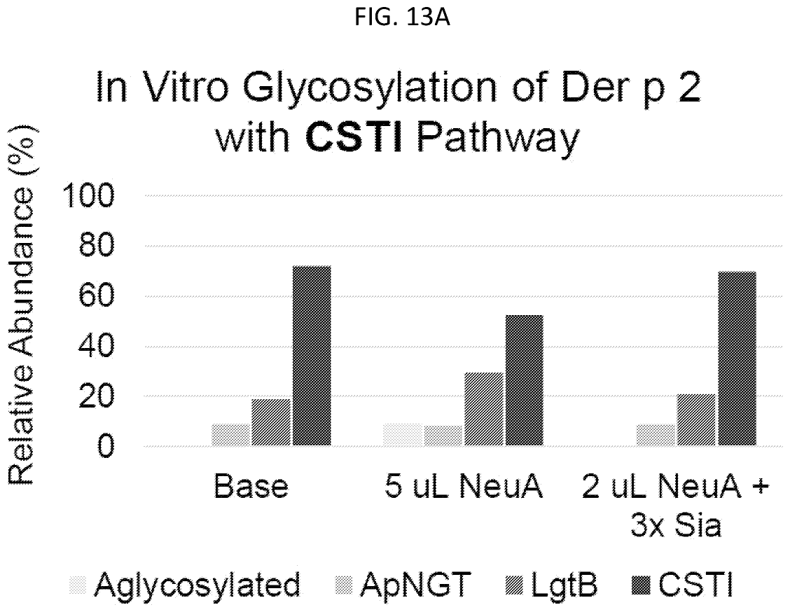

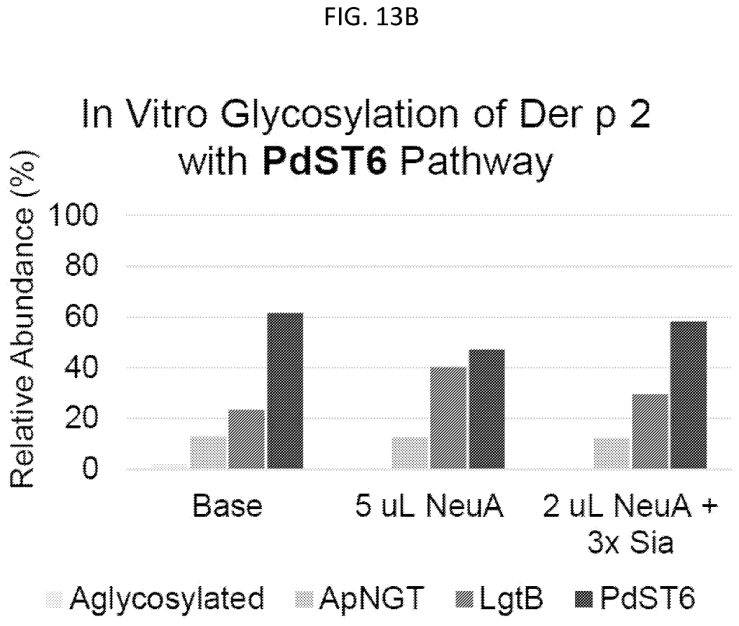

Supplementation of additional sugar and nucleotide substrate matches yields to “base conditions.” Allergen protein Der p 2 was glycosylated in vitro with either the CSTI pathway or the PdST6 pathway in manner similar to that described above with reference to Example 1. The results are shown in FIGS. 13A and 13B. With addition of 2 μL of CFPS lysate expressing NeuA and triple the concentration of sialic acid and CTP (to 7.5 mM) in IVA, the yield of sialylated product approaches the optimal value in “base conditions” in IVA's containing either CSTI pathway components or PdST6 pathway components.

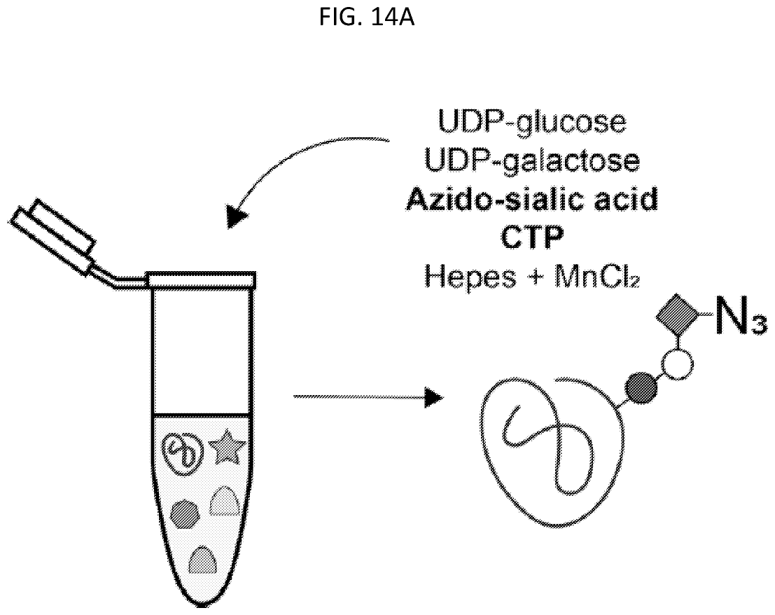

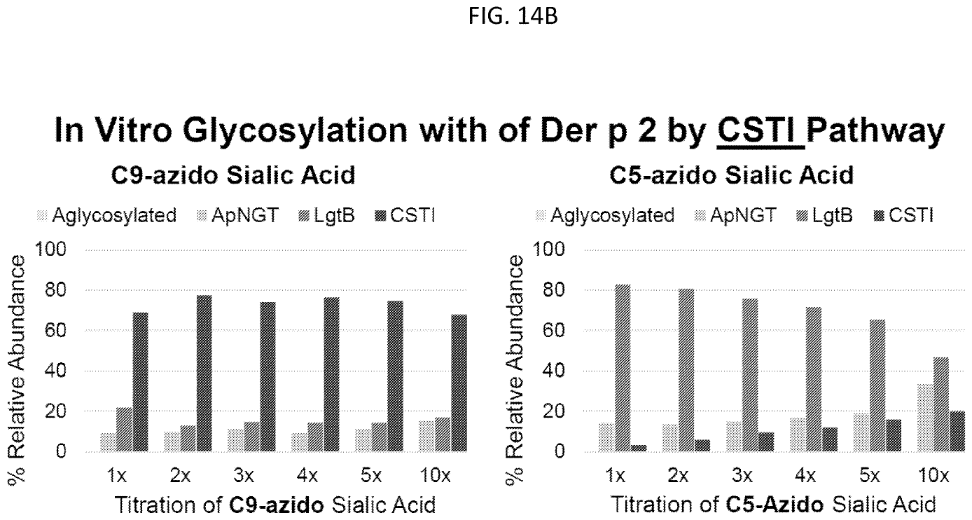

Titration of Non-Canonical Sialic Acid in IVA with CSTI Pathway. FIG. 14A shows a schematic of an in vitro glycosylation of Der p 2 where the azido-sialic acid was supplemented into the IVA in place of standard sialic acid to allow for incorporation of this clickable sialic acid into the glycan structure. This was performed in a similar manner to that described above with respect to Example 1. The results appear in FIG. 14B. The azido-sialic acid had an azide group either at the carbon 9 position (left) or carbon 5 position (right) Supplementation of C9-azido sialic acid at 2× the concentration of standard sialic acid promotes highest yield of sialylated product. Supplementation of C5-azido sialic acid at 10× the concentration yields highest yield of sialylated product. Yields are much lower for incorporation of C5-azido sialic acid than for C9-azido sialic acid, presumably due to reduced permissivity of NeuA, or CSTI to the additional chemical group at this position of its substrate.

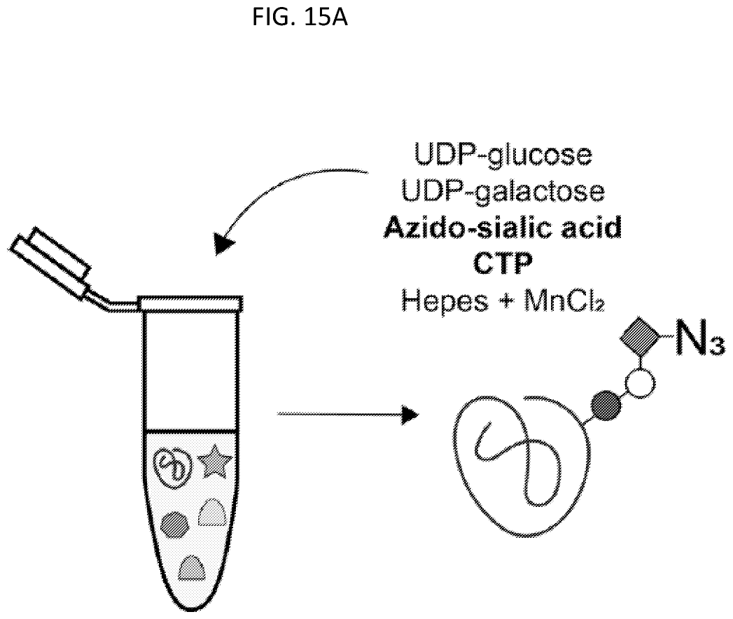

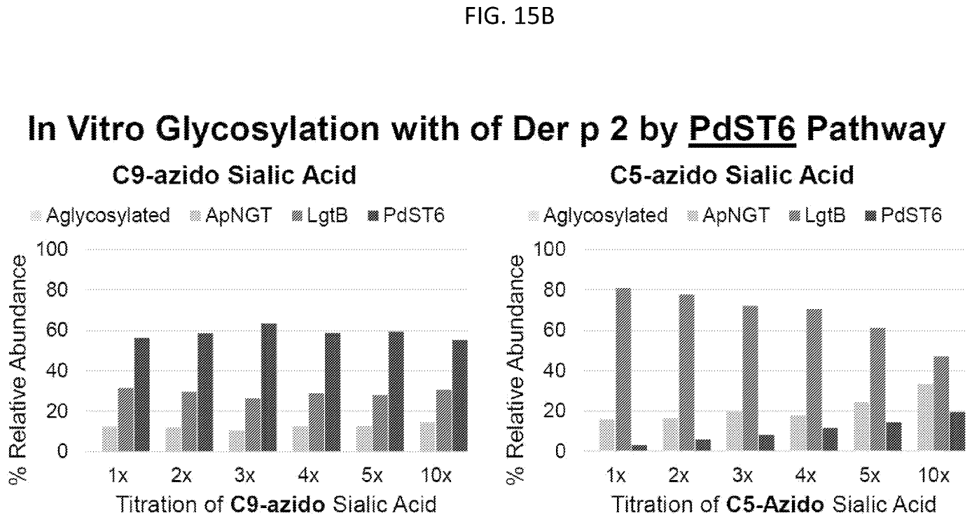

Titration of Non-Canonical Sialic Acid in IVA with PdST6 Pathway. FIG. 15A shows a schematic of an in vitro glycosylation of Der p 2 where the azido-sialic acid was supplemented into the IVA in place of standard sialic acid to allow for incorporation of this clickable sialic acid into the glycan structure. This was performed in a similar manner to that described above with respect to Example 1. The results appear in FIG. 15B. The azido-sialic acid had an azidegroup either at the carbon 9 position (left) or carbon 5 position (right). Supplementation of C9-azido sialic acid at 3× the concentration of standard sialic acid promotes highest yield of sialylated product in the PAST6 pathway. Supplementation of C5-azido sialic acid at 10× the concentration yields highest yield of sialylated product. Yields are much lower for incorporation of C5-azido sialic acid than for C9-azido sialic acid, presumably due to reduced permissivity of NeuA, or PdST6 to the additional chemical group at this position of its substrate.

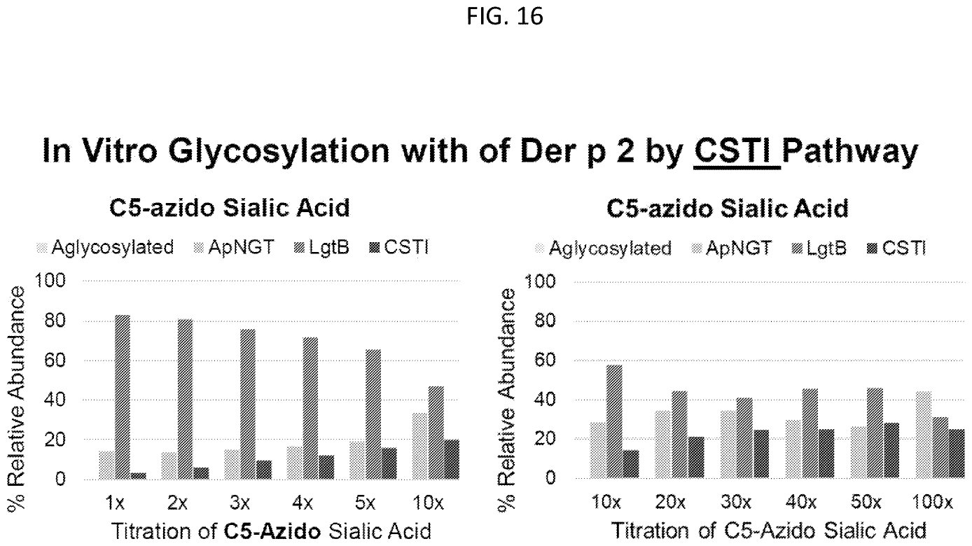

Higher supplementation of C5-azido sialic acid improves yields of sialylated product in the CSTI pathway. In vitro glycosylation of Der p 2 was conducted in a manner similar that described above, with a higher concentration of C5-azido sialic acid. The results are in FIG. 16. Supplementation of C5-azido sialic acid at 50× the concentration of standard sialic acid in base conditions yields the highest yield of sialylated product in the CSTI pathway.

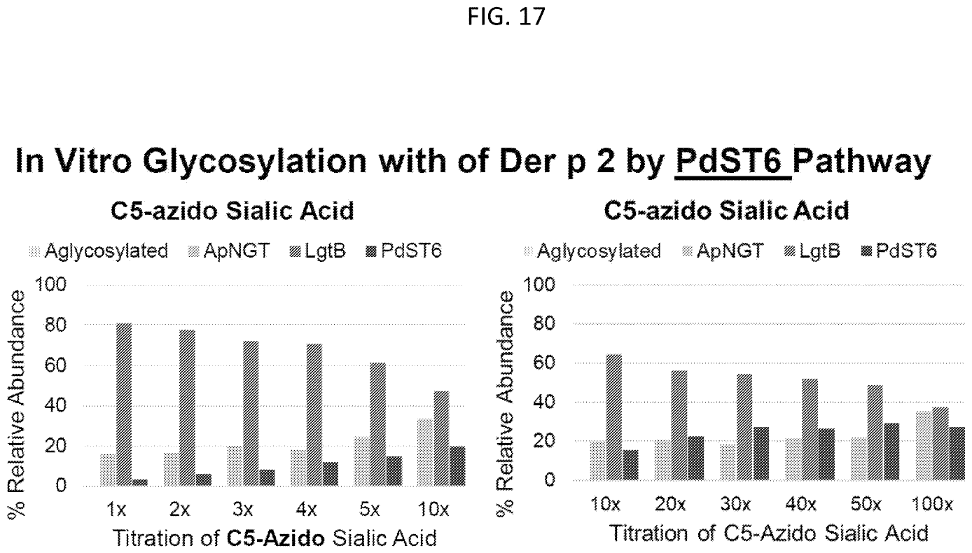

Higher supplementation of C5-azido sialic acid improves yields of sialylated product in the PAST6 pathway. In vitro glycosylation of Der p 2 was conducted in a manner similar that described above, with a higher concentration of C5-azido sialic acid. The results are in FIG. 17. Supplementation of C5-azido sialic acid at 50× the concentration of standard sialic acid in base conditions yields the highest yield of sialylated product in the PAST6 pathway.

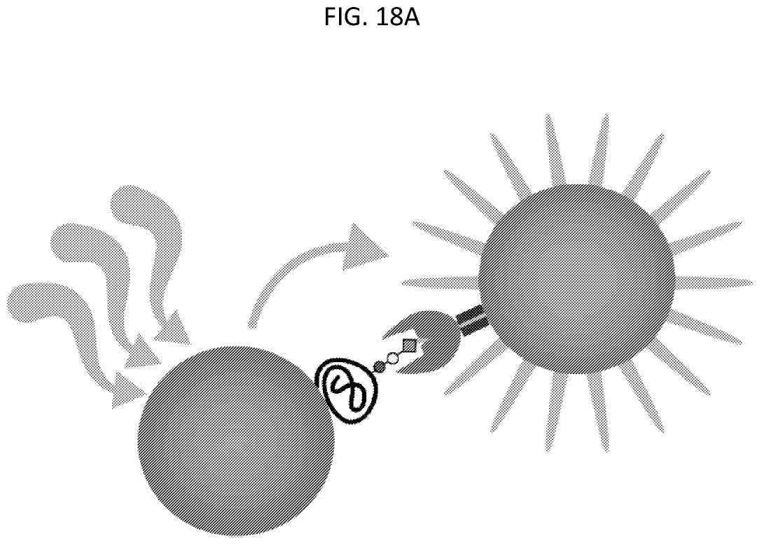

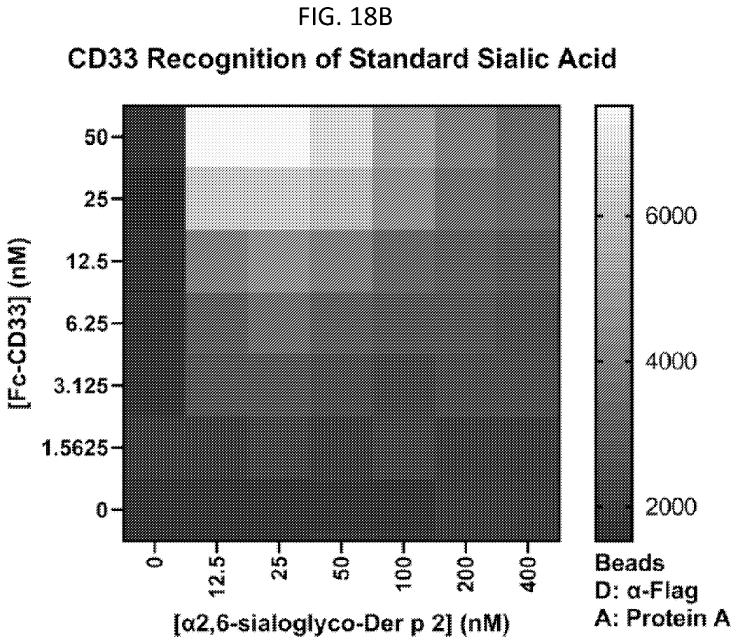

Siglec recognition of GlycoCAP installed glycan. FIG. 18A depicts a schematic of the AlphaLISA assay used in this experiment. The AlphaLISA assay was used to assess binding of Siglec-3 (CD33) to the sialoglycan installed with the GlycoCAP platform. The AlphaLISA involves a donor bead that associates with one binding partner via an affinity tag and an acceptor bead that associates with the other binding partner. If the two entities bind, they will bring the beads in close proximity and an energy transfer will occur causing an emission of light that can be read on a plate reader. CD33 and Der p 2 were co-titrated to determine optimal concentration for detection of binding. At 50 nMCD33 and 12.5 nMα2,6-sialoglyco-Der p 2, there is the highest signal over background indicating that CD33 does indeed recognize the glycan and does so at these optimal concentrations. FIG. 18B shows the results. This experiment confirms that the glycan installed retains expected functional activity.

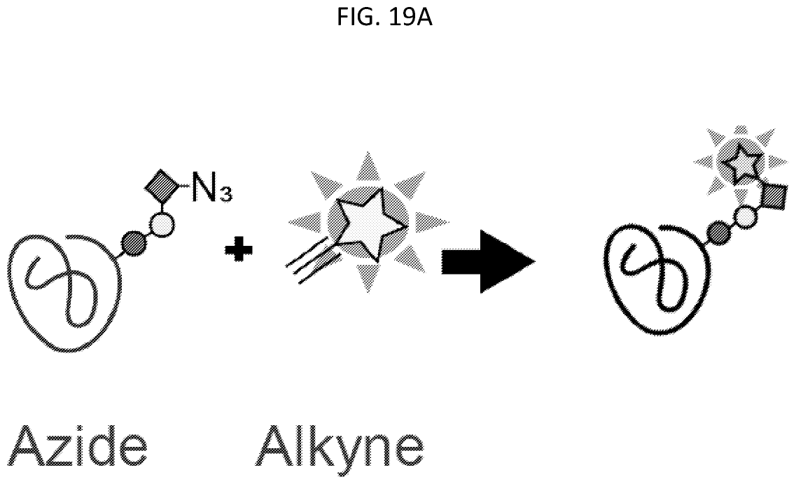

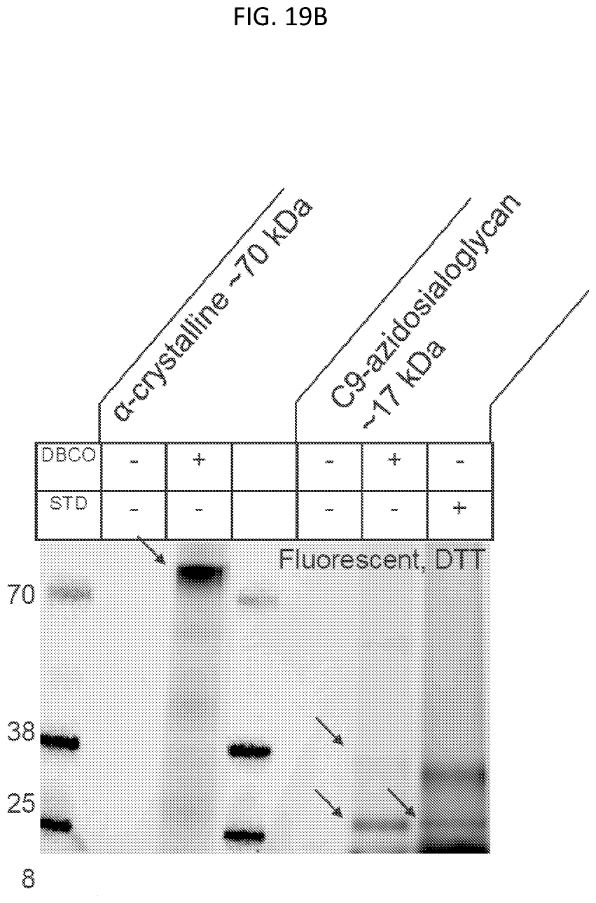

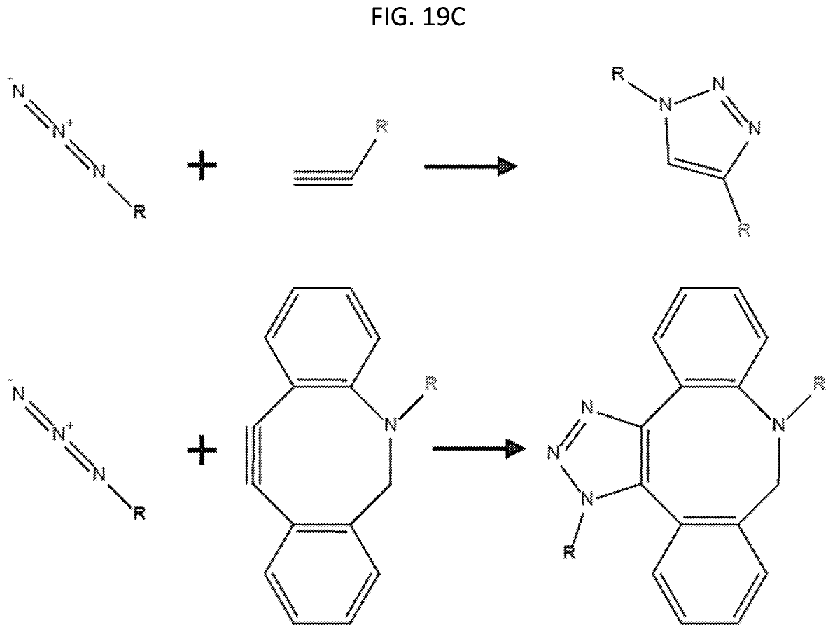

Click conjugation of clickable azido-sialoglycoprotein product from GlycoCAP. FIG. 19A depicts a schematic showing click conjugation of clickable azido-sialoglycoprotein product from GlycoCAP. An alkyne-containing fluorophore was conjugated to the azido-sialoglycoprotein and detected on a fluorescent gel (see FIG. 19B). Lane 1=ladder, lane 2=negative control, lane 3=positive control, lane 4=ladder, lane 5=GlycoCAP-constructed azido-sialoglycoproteinwith no alkyne-containing moiety provided, lane 6=GlycoCAP-constructed azido-sialoglycoprotein with a strained alkyne (DBCO)-containing fluorophore provided, lane 7=GlycoCAP-constructed azido-sialoglycoprotein with a standard alkyne-containing fluorophore provided. The presence of a fluorescent band at the expected mass (~70 kDa for α-crystalline and 17 kDa for C9-azido-sialoglyco-Der p 2) when a fluorophore is provided indicates successful click conjugation of the fluorophore onto the protein. The band at ~34 kDa indicates the presence of dimer formation, which appears to be enhanced by the conditions for the click chemistry reactions with the standard alkyne. FIG. 19C depicts click chemistry conjugation with either a standard (top) or strained (DBCO) (bottom) alkyne results in conjugation of the azide-containing group and alkyne-containing group with the intervening structures shown.

### REFERENCES FOR EXAMPLE 1

(1) Ghannadan, M.; Hauswirth, A. W.; Schernthaner, G. H.; Müller, M. R.; Klepetko, W.; Schatzl, G.; Sperr, W. R.; Bühring, H. J.; Valent, P. Detection of Novel CD Antigens on the Surface of Human Mast Cells and Basophils. Int. Arch. Allergy Immunol. 2002, 127, 299-307.
(2) Yokoi, H.; Choi, O. H.; Hubbard, W.; Lee, H. S.; Canning, B. J.; Lee, H. H.; Ryu, S. D.; von Gunten, S.; Bickel, C. A.; Hudson, S. A.; Macglashan, D. W., Jr.; Bochner, B. S. Inhibition of FcepsilonRI-dependent mediator release and calcium flux from human mast cells by sialic acid-binding immunoglobulin-like lectin 8 engagement. J Allergy Clin Immunol 2008, 121, 499.
(3) O'Reilly, M. K.; Paulson, J. C. Siglecs as targets for therapy in immune-cell-mediated disease. Trends Pharmacol. Sci. 2009, 30, 240-248. (4) Mizrahi, S.; Gibbs, B. F.; Karra, L.; Ben-Zimra, M.; Levi-Schaffer, F. Siglec-7 is an inhibitory receptor on human mast cells and basophils. J Allergy Clin Immunol 2014, 134, 230-233.e3.
(5) Duan, S.; Koziol-White, C. J.; Jester, W. F., Jr.; Nycholat, C. M.; Macauley, M. S.; Panettieri, R. A., Jr.; Paulson, J. C. CD33 recruitment inhibits IgE-mediated anaphylaxis and desensitizes mast cells to allergen. J. Clin. Invest 2019, 129, 1387-1401.
(6) Altrichter, S.; Staubach, P.; Pasha, M.; Rasmussen, H. S.; Singh, B.; Chang, A. T.; Bernstein, J. A.; Siebenhaar, F.; Maurer, M. Efficacy and safety data of AK002, an anti-Siglec-8 monoclonal antibody, in patients with multiple forms of uncontrolled chronic urticaria (CU): results from an open-label phase 2a study. Allergy 2019, 70, 120.
(7) Nycholat, C. M.; Duan, S.; Knuplez, E.; Worth, C.; Elich, M.; Yao, A.; O'Sullivan, J.; McBride, R.; Wei, Y.; Fernandes, S. M.; Zhu, Z.; Schnaar, R. L.; Bochner, B. S.; Paulson, J. C. A Sulfonamide Sialoside Analogue for Targeting Siglec-8 and -F on Immune Cells. J. Am. Chem. Soc. 2019, 141, 14032-14037.
(8) Kerr, S. C.; Gonzalez, J. R.; Schanin, J.; Peters, M. C.; Lambrecht, B. N.; Brock, E. C.; Charbit, A.; Ansel, K. M.; Youngblood, B. A.; Fahy, J. V. An anti-siglec-8 antibody depletes sputum eosinophils from asthmatic subjects and inhibits lung mastcells. Clin. Exp. Allergy 2020, 50, 904-914.
(9) Schanin, J.; Gebremeskel, S.; Korver, W.; Falahati, R.; Butuci, M.; Haw, T. J.; Nair, P. M.; Liu, G.; Hansbro, N. G.; Hansbro, P. M.; Evensen, E.; Brock, E. C.; Xu, A.; Wong, A.; Leung, J.; Bebbington, C.; Tomasevic, N.; Youngblood, B. A. A monoclonal antibody toSiglec-8 suppresses non-allergic airway inflammation and inhibits IgE-independent mast cell activation. Mucosal Immunol. 2021, 14, 366.
(10) Dellon. E. S.; Peterson, K. A.; Murray, J. A.; Falk, G. W.; Gonsalves, N.; Chehade, M.; Genta, R. M.; Leung, J.; Khoury, P.; Klion, A. D.; Hazan, S.; Vaezi, M.; Bledsoe, A. C.; Durrani, S. R.; Wang, C.; Shaw, C.; Chang, A. T.; Singh, B.; Kamboj, A. P.; Rasmussen, H. S.; Rothenberg, M. E.; Hirano, I. Anti-Siglec-8 Antibody for Eosinophilic Gastritis and Duodenitis. N. Engl. J. Med. 2020, 383, 1624-1634.
(11) Duan, S.; Arlian, B. M.; Nycholat, C. M.; Wei, Y.; Tateno, H.; Smith, S. A.; Macauley, M. S.; Zhu, Z.; Bochner, B. S.; Paulson, J. C. Nanoparticles Displaying Allergen and Siglec-8 Ligands Suppress IgE-FcepsilonRI-Mediated Anaphylaxis and Desensitize Mast Cells to Subsequent Antigen Challenge. J. Immunol. 2021, 206, 2290-2300.
(12) Islam, M.; Arlian, B. M.; Pfrengle, F.; Duan, S.; Smith, S. A.; Paulson, J. C. Suppressing Immune Responses Using Siglec Ligand-Decorated Anti-receptor Antibodies. J. Am. Chem. Soc. 2022, 144, 9302-9311.
(13) Bochner, B. S.; O'Sullivan, J. A.; Chang, A. T.; Youngblood, B. A. Siglecs in allergy and asthma. Mol. Aspect. Med. 2023, 90, 101104.
(14) Anesi, S. D.; Tauber, J.; Nguyen, Q. D.; Chang, P.; Berdy, G. J.; Lin, C. C.; Chu, D. S.; Levine, H. T.; Fernandez, A. D.; Roy, N.; Asbell, P. A.; Kantor, A. M.; Chang, A. T.; Singh, B.; Youngblood, B. A.; Jeng, B. H.; Jhanji, V.; Rasmussen, H. S.; Foster, C. S. Lirentelimab for severe and chronic forms of allergic conjunctivitis. J Allergy Clin Immunol 2022, 150, 631-639.
(15) Paul, S. P.; Taylor, L. S.; Stansbury, E. K.; McVicar, D. W. Myeloid specific human CD33 is an inhibitory receptor with differential ITIM function in recruiting the phosphatases SHP-1 and SHP-2. Blood 2000, 96, 483-490.
(16) Macauley, M. S.; Crocker, P. R.; Paulson, J. C. Siglec-mediated regulation of immune cell function in disease. Nat. Rev. Immunol. 2014, 14, 653-666.
(17) Hernandez-Caselles, T.; Miguel, R. C.; Ruiz-Alcaraz, A. J.; Garcia-Penarrubia, P. CD33 (Siglec-3) Inhibitory Function: Role in the NKG2D/DAP10 Activating Pathway. J. Immunol. Res. 2019, 2019, 6032141.
(18) O'Sullivan, J. A.; Chang, A. T.; Youngblood, B. A.; Bochner, B. S. Eosinophil and mast cell Siglecs: From biology to drug target. J. Leukoc. Biol. 2020, 108, 73-81.
(19) Duan, S.; Paulson, J. C. Siglecs as Immune Cell Checkpoints in Disease. Annu. Rev. Immunol. 2020, 38, 365-395.
(20) North, S. J.; Huang, H. H.; Sundaram, S.; Jang-Lee, J.; Etienne, A. T.; Trollope, A.; Chalabi, S.; Dell, A.; Stanley, P.; Haslam, S. M. Glycomics profiling of Chinese hamster ovary cell glycosylation mutants reveals N-glycans of a novel size and complexity. J. Biol. Chem. 2010, 285, 5759-5775.
(21) Meuris, L.; Santens, F.; Elson, G.; Festjens, N.; Boone, M.; Dos Santos, A.; Devos, S.; Rousseau, F.; Plets, E.; Houthuys, E.; Malinge, P.; Magistrelli, G.; Cons, L.; Chatel, L.; Devreese, B.; Callewaert, N. GlycoDelete engineering of mammalian cells simplifies N-glycosylation of recombinant proteins. Nat. Biotechnol. 2014, 32, 485-489.
(22) Yang. Z.; Wang, S.; Halim, A.; Schulz, M. A.; Frodin, M.; Rahman, S. H.; Vester-Christensen, M. B.; Behrens, C.; Kristensen, C.; Vakhrushev, S. Y.; Bennett, E. P.; Wandall, H. H.; Clausen, H. Engineered CHO cells for production of diverse, homogeneous glycoproteins. Nat. Biotechnol. 2015, 33, 842-844.
(23) Johannssen, T.; Lepenies, B. Glycan-Based Cell Targeting To Modulate Immune Responses. Trends Biotechnol. 2017, 35, 334-346.
(24) Wang, L. X.; Amin, M. N. Chemical and chemoenzymatic synthesis of glycoproteins for deciphering functions. Chem. Biol. 2014, 21, 51-66.
(25) Rodrigues, E.; Jung, J.; Park, H.; Loo, C.; Soukhtehzari, S.; Kitova, E. N.; Mozaneh, F.; Daskhan, G.; Schmidt, E. N.; Aghanya, V.; Sarkar, S.; Streith, L.; St Laurent, C. D.; Nguyen, L.; Julien, J. P.; West, L. J.; Williams, K. C.; Klassen, J. S.; Macauley, M. S. A versatile soluble siglec scaffold for sensitive and quantitative detection of glycan ligands. Nat. Commun. 2020, 11, 5091.
(26) Jung, J.; Enterina, J. R.; Bui, D. T.; Mozanch, F.; Lin, P. H.; NitinKuo, C. W.; Kuo, C. W.; Rodrigues, E.; Bhattacherjee, A.; Raeisimakiani, P.; Daskhan, G. C.; St Laurent, C. D.; Khoo, K. H.; Mahal, L. K.; Zandberg, W. F.; Huang, X.; Klassen, J. S.; et al. Carbohydrate Sulfation As a Mechanism for Fine-Tuning Siglec Ligands. ACS Chem. Biol. 2021, 16, 2673-2689.
(27) Hershewe, J.; Kightlinger, W.; Jewett, M. C. Cell-free systems for accelerating glycoprotein expression and biomanufacturing. J. Ind. Microbiol. Biotechnol. 2020, 47, 977-991.
(28) Jaroentomeechai, T.; Taw, M. N.; Li, M.; Aquino, A.; Agashe, N.; Chung, S.; Jewett, M. C.; DeLisa, M. P. Cell-Free Synthetic Glycobiology: Designing and Engineering Glycomolecules Outside of Living Cells. Front. Chem. 2020, 8, 645.
(29) Kightlinger, W.; Warfel, K. F.; DeLisa, M. P.; Jewett, M. C. Synthetic Glycobiology: Parts, Systems, and Applications. ACS Synth. Biol. 2020, 9, 1534-1562.
(30) Martin, R. W.; Des Soye, B. J.; Kwon, Y. C.; Kay, J.; Davis, R. G.; Thomas, P. M.; Majewska, N. I.; Chen, C. X.; Marcum, R. D.; Weiss, M. G.; Stoddart, A. E.; Amiram, M.; Charna, A. K. R.; Patel, J. R.; Isaacs, F. J.; Kelleher, N. L.; Hong, S. H.; Jewett, M. C. Cell-free protein synthesis from genomically recoded bacteria enables multisite incorporation of noncanonical amino acids. Nat. Commun. 2018, 9, 1203.
(31) Zawada, J. F.; Yin, G.; Steiner, A. R.; Yang, J.; Naresh, A.; Roy, S. M.; Gold, D. S.; Heinsohn, H. G.; Murray, C. J. Microscale to manufacturing scale-up of cell-free cytokine production—a new approach for shortening protein production development timelines Biotechnol. Bioeng. 2011, 108, 1570-1578.
(32) Silverman, A. D.; Karim, A. S.; Jewett, M. C. Cell-free gene expression: an expanded repertoire of applications. Nat. Rev. Genet. 2020, 21, 151-170.
(33) Jaroentomeechai, T.; Zheng, X.; Hershewe, J.; Stark, J. C.; Jewett, M. C.; DeLisa, M. P. A Pipeline for Studying and Engineering Single-Subunit Oligosaccharyltransferases. Methods Enzymol. 2017, 597, 55-81.
(34) Jaroentomeechai, T.; Stark, J. C.; Natarajan, A.; Glasscock, C. J.; Yates, L. B.; Hsu, K. J.; Mrksich, M.; Jewett, M. C.; DeLisa, M. P. Single-pot glycoprotein biosynthesis using a cell-free transcriptiontranslation system enriched with glycosylation machinery. Nat. Commun. 2018, 9, 2686.
(35) Schoborg, J. A.; Hershewe, J. M.; Stark, J. C.; Kightlinger, W.; Kath, J. E.; Jaroentomeechai, T.; Natarajan, A.; DeLisa, M. P.; Jewett, M. C. A cell-free platform for rapid synthesis and testing of active oligosaccharyltransferases. Biotechnol. Bioeng. 2018, 115, 739-750.
(36) Natarajan, A.; Jaroentomeechai, T.; Cabrera-Sanchez, M.; Mohammed, J. C.; Cox, E. C.; Young, O.; Shajahan, A.; Vilkhovoy, M.; Vadhin, S.; Varner, J. D.; Azadi, P.; DeLisa, M. P. Engineering orthogonal human O-linked glycoprotein biosynthesis in bacteria. Nat. Chem. Biol. 2020, 16, 1062-1070.
(37) Hershewe, J. M.; Warfel, K. F.; Iyer, S. M.; Peruzzi, J. A.; Sullivan, C. J.; Roth, E. W.; DeLisa, M. P.; Kamat, N. P.; Jewett, M. C. Improving cell-free glycoprotein synthesis by characterizing and enriching native membrane vesicles. Nat. Commun. 2021, 12, 2363.
(38) Stark, J. C.; Jaroentomeechai, T.; Moeller, T. D.; Hershewe, J. M.; Warfel, K. F.; Moricz, B. S.; Martini, A. M.; Dubner, R. S.; Hsu, K. J.; Stevenson, T. C.; Jones, B. D.; DeLisa, M. P.; Jewett, M. C. On-demand biomanufacturing of protective conjugate vaccines. Sci. Adv. 2021, 7, No. eabe9444.
(39) Jaroentomeechai, T.; Kwon, Y. H.; Liu, Y.; Young, O.; Bhawal, R.; Wilson, J. D.; Li, M.; Chapla, D. G.; Moremen, K. W.; Jewett, M. C.; Mizrachi, D.; DeLisa, M. P. A universal glycoenzyme biosynthesis pipeline that enables efficient cell-free remodeling of glycans. Nat. Commun. 2022, 13, 6325.
(40) Warfel, K. F.; Williams, A.; Wong, D. A.; Sobol, S. E.; Desai, P.; Li, J.; Chang, Y. F.; DeLisa, M. P.; Karim, A. S.; Jewett, M. C. A Low-Cost, Thermostable, Cell-Free Protein Synthesis Platform for On-Demand Production of Conjugate Vaccines. ACS Synth. Biol. 2023, 12, 95-107.
(41) Williams, A. J.; Warfel, K. F.; Desai, P.; Li, J.; Lee, J.-J.; Wong, D. A.; Nguyen, P. M.; Qin, Y.; Sobol, S. E.; Jewett, M. C.; Chang, Y.-F.; DeLisa, M. P. A low-cost recombinant glycoconjugate vaccine confers immunogenicity and protection against enterotoxigenic Escherichia coli infections in mice. Front. Mol. Biosci. 2023, 10, 2022.
(42) Warfel, K. F.; Laigre, E.; Sobol, S. E.; Gillon, E.; Varrot, A.; Renaudet, O.; Dejeu, J.; Jewett, M. C.; Imberty, A. Cell-free expression and characterization of multivalent rhamnose-binding lectins using biolayer interferometry. Glycobiology 2023, DOI: 10.1093/glycob/cwad018
(43) Kightlinger, W.; Lin, L.; Rosztoczy, M.; Li, W.; DeLisa, M. P.; Mrksich, M.; Jewett, M. C. Design of glycosylation sites by rapid synthesis and analysis of glycosyltransferases. Nat. Chem. Biol. 2018, 14, 627-635.
(44) Lin, L.; Kightlinger, W.; Prabhu, S. K.; Hockenberry, A. J.; Li, C.; Wang, L. X.; Jewett, M. C.; Mrksich, M. Sequential Glycosylation of Proteins with Substrate-Specific N-Glycosyltransferases. ACS Cent. Sci. 2020, 6, 144-154.
(45) Kightlinger, W.; Duncker, K. E.; Ramesh, A.; Thames, A. H.; Natarajan, A.; Stark, J. C.; Yang, A.; Lin, L.; Mrksich, M.; DeLisa, M. P.; Jewett, M. C. A cell-free biosynthesis platform for modular construction of protein glycosylation pathways. Nat. Commun. 2019, 10, 5404.
(46) Kolb, H. C.; Finn, M. G.; Sharpless, K. B. Click Chemistry: Diverse Chemical Function from a Few Good Reactions. Angew. Chem., Int. Ed. 2001, 40, 2004-2021.
(47) Sletten, E. M.; Bertozzi, C. R. Bioorthogonal chemistry: fishing for selectivity in a sea of functionality. Angew. Chem., Int. Ed. Engl. 2009, 48, 6974-6998.
(48) Baskin, J. M.; Bertozzi, C. R. Bioorthogonal Click Chemistry: Covalent Labeling in Living Systems. QSAR Comb. Sci. 2007, 26, 1211-1219.
(49) Rillahan, C. D.; Schwartz, E.; McBride, R.; Fokin, V. V.; Paulson, J. C. Click and pick: identification of sialoside analogues for Siglec-based cell targeting. Angew. Chem., Int. Ed. Engl. 2012, 124, 11176-11180.
(50) Macauley, M. S.; Pfrengle, F.; Rademacher, C.; Nycholat, C. M.; Gale, A. J.; von Drygalski, A.; Paulson, J. C. Antigenic liposomes displaying CD22 ligands induce antigen-specific B cell apoptosis. J. Clin. Invest. 2013, 123, 3074-3083
(51) Rillahan, C. D.; Macauley, M. S.; Schwartz, E.; He, Y.; McBride, R.; Arlian, B. M.; Rangarajan, J.; Fokin, V. V.; Paulson, J. C. Disubstituted Sialic Acid Ligands Targeting Siglecs CD33 and CD22 Associated with Myeloid Leukaemias and B Cell Lymphomas. Chem. Sci. 2014, 5, 2398-2406.
(52) Bull, C.; Heise, T.; van Hilten, N.; Pijnenborg, J. F. A.; Bloemendal, V. R. L. J.; Gerrits, L.; Kers-Rebel, E. D.; Ritschel, T.; den Brok, M. H.; Adema, G. J.; Boltje, T. J. Steering Siglec-Sialic Acid Interactions on Living Cells using Bioorthogonal Chemistry. Angew. Chem., Int. Ed. Engl. 2017, 129, 3357-3361.
(53) Baskin, J. M.; Prescher, J. A.; Laughlin, S. T.; Agard, N. J.; Chang, P. V.; Miller, I. A.; Lo, A.; Codelli, J. A.; Bertozzi, C. R. Copper-free click chemistry for dynamic in vivo imaging. Proc. Natl. Acad. Sci. U.S.A 2007, 104, 16793-16797.
(54) Hudak, J. E.; Barfield, R. M.; de Hart, G. W.; Grob, P.; Nogales, E.; Bertozzi, C. R.; Rabuka, D. Synthesis of heterobifunctional protein fusions using copper-free click chemistry and the aldehyde tag. Angew. Chem., Int. Ed. Engl. 2012, 124, 4237-4241.
(55) Dehnert, K. W.; Baskin, J. M.; Laughlin, S. T.; Beahm, B. J.; Naidu, N. N.; Amacher, S. L.; Bertozzi, C. R. Imaging the sialome during zebrafish development with copper-free click chemistry. Chembiochem 2012, 13, 353-357.
(56) Des Soye, B. J.; Gerbasi, V. R.; Thomas, P. M.; Kelleher, N. L.; Jewett, M. C. A Highly Productive, One-Pot Cell-Free Protein Synthesis Platform Based on Genomically Recoded Escherichia coli. Cell Chem. Biol. 2019, 26, 1743-1754.e9.
(57) Naegeli, A.; Neupert, C.; Fan, Y. Y.; Lin, C. W.; Poljak, K.; Papini, A. M.; Schwarz, F.; Aebi, M. Molecular analysis of an alternative N-glycosylation machinery by functional transfer from Actinobacillus pleuropneumoniae to Escherichia coli. J. Biol. Chem. 2014, 289, 2170-2179.
(58) Schwarz, F.; Fan, Y. Y.; Schubert, M.; Aebi, M. Cytoplasmic N-glycosyltransferase of Actinobacillus pleuropneumoniae is an inverting enzyme and recognizes the NX(S/T) consensus sequence. J. Biol. Chem. 2011, 286, 35267-35274.
(59) Chua, K. Y.; Doyle, C. R.; Simpson, R. J.; Turner, K. J.; Stewart, G. A.; Thomas, W. R. Isolation of cDNA Coding for the Major Mite Allergen Der p II by IgE Plaque Immunoassay. Int. Arch. Allergy Immunol. 1990, 91, 118-123.
(60) Derewenda, U.; Li, J.; Derewenda, Z.; Dauter, Z.; Mueller, G.; Rule, G.; Benjamin, D. The Crystal Structure of a Major Dust Mite Allergen Der p 2, and its Biological Implications. J. Mol. Biol. 2002, 318, 189-197.
(61) Miles, L. A.; Hermans, S. J.; Crespi, G. A. N.; Gooi, J. H.; Doughty, L.; Nero, T. L.; Markulic, J.; Ebneth, A.; Wroblowski, B.; Oehlrich, D.; Trabanco, A. A.; Rives, M. L.; Royaux, I.; Hancock, N. C.; Parker, M. W. Small Molecule Binding to Alzheimer Risk Factor CD33 Promotes Abeta Phagocytosis. iScience 2019, 19, 110-118.
(62) Landolina, N.; Zaffran, I.; Smiljkovic, D.; Serrano-Candelas, E.; Schmiedel, D.; Friedman, S.; Arock, M.; Hartmann, K.; Pikarsky, E.; Mandelboim, O.; Martin, M.; Valent, P.; Levi-Schaffer, F. Activation of Siglec-7 results in inhibition of in vitro and in vivo growth of human mast cell leukemia cells. Pharmacol. Res. 2020, 158, 104682.
(63) Wisnovsky, S.; Mockl, L.; Malaker, S. A.; Pedram, K.; Hess, G. T.; Riley, N. M.; Gray, M. A.; Smith, B. A. H.; Bassik, M. C.; Moerner, W. E.; Bertozzi, C. R. Genome-wide CRISPR screens reveal a specific ligand for the glycan-binding immune checkpoint receptor Siglec-7. Proc. Natl. Acad. Sci. U.S.A 2021, 118, DOI: 10.1073/pnas.2015024118.
(64) Han, S.; Collins, B. E.; Bengtson, P.; Paulson, J. C. Homomultimeric complexes of CD22 in B cells revealed by protein-glycan cross-linking. Nat. Chem. Biol. 2005, 1, 93-97.
(65) Swartz, J. R.; Jewett, M. C.; Woodrow, K. A. Cell-Free Protein Synthesis With Prokaryotic Combined Transcription-Translation. In Methods in Molecular Biology; Balbás, P., Lorence, A., Eds.; Humana Press: Totowa, NJ, 2004, pp 169-182

### ADDITIONAL REFERENCES

### Patents

WO2022002918A1-One-pot cell-free glycosylation process
U.S. Pat. No. 6,780,607 Method for cell-free protein complete post-translational modification
U.S. Pat. No. 11,193,154 Prokaryote-based cell-free system for the synthesis of glycoproteins
U.S. Pat. No. 10,994,006 Desensitizing mast cells by co-presentation of antigens with high affinity mast cell siglec ligands
U.S. Pat. No. 7,220,555 Practical sialylation of recombinant glycoproteins
U.S. Pat. No. 6,399,336 Practical in vitro sialylation of recombinant glycoproteins
U.S. Pat. No. 5,409,817 Use of trans-sialidase and sialyltransferase for synthesis of sialyl.alpha.2 fwdarw. 3.beta.galactosides
CA3127668A1 Modular platform for producing glycoproteins and identifying glycosylation pathways

### PUBLICATIONS

1. Bull, C., T. Heise, N. van Hilten, J. F. Pijnenborg, V. R. Bloemendal, L. Gerrits, E. D. Kers-Rebel, T. Ritschel, M. H. den Brok, G. J. Adema, and T. J. Boltje, Steering Siglec-Sialic Acid Interactions on Living Cells using Bioorthogonal Chemistry. Angew Chem Int Ed Engl, 2017. 56(12): p. 3309-3313.
2. Nycholat, C. M., S. Duan, E. Knuplez, C. Worth, M. Elich, A. Yao, J. O'Sullivan, R. McBride, Y. Wei, S. M. Fernandes, Z. Zhu, R. L. Schnaar, B. S. Bochner, and J. C. Paulson, A Sulfonamide Sialoside Analogue for Targeting Siglec-8 and -F on Immune Cells. J Am Chem Soc, 2019. 141(36): p. 14032-14037. PMC6861165
3. Kightlinger, W., K. E. Duncker, A. Ramesh, A. H. Thames, A. Natarajan, J. C. Stark, A. Yang, L. Lin, M. Mrksich, M. P. DeLisa, and M. C. Jewett, A cell-free biosynthesis platform for modular construction of protein glycosylation pathways. Nat Commun, 2019. 10(1): p. 5404. PMC6881289
4. Duan, S., C. J. Koziol-White, W. F. Jester, Jr., C. M. Nycholat, M. S. Macauley, R. A. Panettieri, Jr., and J. C. Paulson, CD33 recruitment inhibits IgE-mediated anaphylaxis and desensitizes mast cells to allergen. J Clin Invest, 2019. 129(3): p. 1387-1401. PMC6391081
5. Johannssen, T. and B. Lepenies, Glycan-Based Cell Targeting To Modulate Immune Responses. Trends Biotechnol, 2017. 35(4): p. 334-346.
6. Rillahan, C. D., E. Schwartz, R. McBride, V. V. Fokin, and J. C. Paulson, Click and pick: identification of sialoside analogues for siglec-based cell targeting. Angew Chem Int Ed Engl, 2012. 51(44): p. 11014-8. PMC3480989
7. Rillahan, C. D., M. S. Macauley, E. Schwartz, Y. He, R. McBride, B. M. Arlian, J. Rangarajan, V. V. Fokin, and J. C. Paulson, Disubstituted Sialic Acid Ligands Targeting Siglecs CD33 and CD22 Associated with Myeloid Leukaemias and B Cell Lymphomas. Chem Sci, 2014. 5(6): p. 2398-2406. PMC4048721
8. Kolb, H. C., Finn, M. G., Sharpless, K. Barry, Click Chemistry: Diverse Chemical Function from a Few Good Reactions. Angewandte Chemie International Edition, 2001. 40: p. 2004-2021.
9. Kightlinger, W., L. Lin, M. Rosztoczy, W. Li, M. P. DeLisa, M. Mrksich, and M. C. Jewett, Design of glycosylation sites by rapid synthesis and analysis of glycosyltransferases. Nat Chem Biol, 2018.
10. Bochner, B. S., R. A. Alvarez, P. Mehta, N. V. Bovin, O. Blixt, J. R. White, and R. L. Schnaar, Glycan array screening reveals a candidate ligand for Siglec-8. J Biol Chem, 2005. 280(6): p. 4307-12.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for preparing an azido-sialoglycoprotein, the method comprising:
in a cell-free in vitro glycosylation assembly (IVA) reaction, providing a polypeptide comprising an amino acid sequence recognized by ApNGT for glycosylation;
wherein the IVA comprises one or more of: ApNGT, LgtB, CSTI, PdST6, NeuA, or CTP.

2. The method of claim 1, wherein the IVA comprises one or more E. coli lysates.

3. The method of claim 2, wherein at least one of the E. coli lysates is derived from a genetically modified E. coli strain.

4. The method of claim 2, wherein the one or more E. coli lysates expresses one or more of ApNGT, LgtB, CSTI, PAST6, or NeuA.

5. The method of claim 1, wherein the IVA comprises one or sugars comprising an azido moiety.

6. The method of claim 5, wherein the one or more sugars comprising an azido moiety is C5 azido-sialic acid, C9-azido sialic acid, or both.

7. The method of claim 1, wherein the polypeptide is expressed in a cell-free protein synthesis system (CFPS).

8. The method of claim 1, wherein the amino acid sequence recognized by ApNGT for glycosylation is GGNWTT (SEQ ID NO: 18).

9. The method of claim 1, wherein the amino acid sequence recognized by ApNGT for glycosylation is attached to the polypeptide by a linker or fusion protein.

10. The method of claim 1, wherein the polypeptide is an allergen protein, a synthetic protein, or an antibody or fragment thereof.

11. The method of claim 1, wherein the polypeptide is an allergen protein, and wherein the allergen protein is one or more of Der p 1; Der p 2; Bos d 5; Bos d 4; Gal d 1; Gal d 2; Gal d 4; Ara h 2; Fel d 1; Amb a 1; Bet v 1; Phl p 5; Bla g 2; or Cry j 1.

12. The method of claim 1, further comprising converting the polypeptide into an azido-sialoglycoprotein.

13. The method of claim 12, conjugating an alkyne-containing moiety to the azido-sialoglycoprotein.

14. A composition comprising the IVA of claim 1.

15. The composition of claim 14, further comprising one or sugars comprising an azido moiety.

16. The composition of claim 15, wherein the one or more sugars comprising an azido moiety is C5 azido-sialic acid, C9-azido sialic acid, or both.

17. A genetically engineered E. coli strain comprising one or more nucleic acid sequences encoding one or more of: ApNGT, LgtB, CSTI, PAST6, or NeuA.

18. The genetically engineered E. coli strain of claim 17, wherein the ApNGT is from Actinobacillus pleuropneumoniae, wherein the LgtB is from Neisseria gonorrhoeae, wherein the CSTI is from Campylobacter jejuni, and wherein the PAST6 is from Photobacterium damselae.

19. A kit comprising the IVA of claim 1.

20. The kit of claim 19, further comprising one or more E. coli lysates, one of sugars comprising an azido moiety, or both.
